SEQUENCE LISTING (5 sequences)

SEQ ID NO: 1 - DNA, 23 bp, Artificial Sequence
  Synthetic primer
ttaagcttcatcagtatccccca

SEQ ID NO: 2 - DNA, 26 bp, Artificial Sequence
  Synthetic primer
caaagtaggaaaacatcatcacagga

SEQ ID NO: 3 - DNA, 17 bp, Artificial Sequence
  Synthetic probe
accatctctaaaatcct

SEQ ID NO: 4 - DNA, 70 bp, Homo sapiens
caaagtaggaaaacatcatcacaggatagaggattttagagatggtatgggggatactgatgaagcttaa

SEQ ID NO: 5 - DNA, 70 bp, Homo sapiens
ttaagcttcatcagtatcccccataccatctctaaaatcctctatcctgtgatgatgttttcctactttg

(19) United States
(12) Patent Application Publication
(10) Pub. No.: US 2016/0362729 A1
(43) Pub. Date: Dec. 15, 2016

(54) METHODS AND COMPOSITIONS FOR CHROMOSOME MAPPING

(71) Applicant: Bio-Rad Laboratories, Inc., Hercules, CA (US)

(72) Inventors: John Frederick Regan, San Mateo, CA (US); Svilen Tzonev, Pleasanton, CA (US)

(21) Appl. No.: 14/498,352

(22) Filed: Sep. 26, 2014

Related U.S. Application Data
(60) Provisional application No. 61/882,969, filed on Sep. 26, 2013.

(51) Int. Cl. C12Q 1/68

(52) U.S. Cl. CPC C12Q 1/6816

(57) ABSTRACT

Provided herein are improved methods, compositions, and kits for analysis of nucleic acids. The improved methods, compositions, and kits can enable directional chromosome mapping e.g., using chromosome phasing/haplotyping. The improved methods, compositions, and kits can also enable copy number estimation of a nucleic acid in a sample. Also provided herein are methods, compositions, and kits for determining the linkage of two or more copies of a target nucleic acid in a sample (e.g., whether the two or more copies are on the same chromosome or different chromosomes) or for phasing alleles.

### CROSS-REFERENCE

This application claims the benefit of U.S. Provisional Application No. 61/882,969, filed Sep. 26, 2013, which application is incorporated herein by reference in its entirety.

### BACKGROUND

Chromosome mapping can be used to determine a location of specific loci (e.g., genes) on a chromosome. In some cases, next generation sequencing can be used for chromosome mapping. However, in some cases, next generation sequencing alone is not sufficient to provide a full understanding of complex chromosomal architecture. For example, in some cases, next generation sequencing based on relatively short sequence reads cannot be used to span a complex rearranged region on a chromosome. A complex rearranged region of a chromosome can comprise copy number variations. Copy number variable (CNV) regions can comprise about 12% of human genomic DNA. These regions can vary from about 1 kb to several megabases in size. CNV regions can be difficult to map on a chromosome.

As recognized herein, improved methods are needed for directional mapping of chromosome elements in complex regions of genomic DNA sequence.

### SUMMARY

In one aspect, a method for determining an arrangement of at least three loci on a first chromosome is provided, the method comprising: obtaining a sample comprising polynucleotide fragments of the first chromosome; partitioning the polynucleotide fragments of the first chromosome; amplifying at least three loci from the polynucleotide fragments of the first chromosome, thereby generating at least three amplified loci of the first chromosome; detecting the at least three amplified loci of the first chromosome with a set of at least three probes, wherein each of the at least three probes comprises a different label; determining linkage frequencies among the at least three loci of the first chromosome; and based on the linkage frequencies, determining the arrangement of the at least three loci on the first chromosome.

In some cases, determining the arrangement of the at least three loci comprises determining a distance between a first locus and a second locus of the at least three loci. In some cases, determining the arrangement of the at least three loci comprises determining a distance between the second locus and a third locus of the at least three loci. In some cases, determining the arrangement of the at least three loci comprises determining a distance between the first locus and the third locus of the at least three loci. In some cases, the distance is a relative distance. In some cases, the distance is determined by comparing the linkage frequencies to a standard. In some cases, the standard is based on linkage frequencies of molecules separated by a known distance. In some cases, determining the arrangement of the at least three loci comprises determining an order of a first locus, second locus, and third locus on the first chromosome.

In some cases, the method further comprises detecting a plurality of amplified loci of the first chromosome with a second set of at least three probes, wherein a first probe of the first set of probes anneals to a first locus, a second probe of the first set anneals to a second locus, a first probe of the second set anneals to the first locus, and a second probe of the second set of probes anneals to the second locus. In some cases, a third probe of the first set of probes anneals to a third locus, and a third probe of the second set of probes anneals to a fourth locus, wherein the third locus and the fourth locus are not the same.

In some cases, the method further comprises detecting the at least three amplified loci of the first chromosome with at least two sets of at least three probes, wherein each probe in each set comprises a different label. In some cases, each set of probes comprises probes with the same labels. In some cases, each set of probes comprises at least three probes, wherein each probe in a set comprises a different label. In some cases, each probe in the at least two sets of probes anneals to a different locus.

In some cases, a first set of at least three probes comprises at least one probe that anneals to the same locus as at least one probe in a second set of at least three probes. In some cases, each probe in each set comprises a different label. In some cases, each set of probes comprises the same labels. In some cases, a first set of at least three probes comprises at least two probes that anneal to the same loci as at least two probes in a second set of at least three probes. In some cases, each of at least three sets of probes comprising at least three probes comprises at least one probe that anneals to the same locus as a probe of the other sets of probes. In some cases, each probe that anneals to the same locus comprises the same label.

In some cases, the sample comprises polynucleotide fragments of a second chromosome, wherein the second chromosome is different from the first chromosome. In some cases, the method further comprises partitioning the polynucleotide fragments of the second chromosome. In some cases, the method further comprises amplifying at least one locus of the second chromosome, thereby generating at least one amplified locus of the second chromosome.

In some cases, the method further comprises detecting the at least one amplified locus on the second chromosome with a reference probe, wherein the reference probe is a fourth probe in the set of at least three probes, wherein the reference probe comprises a label different than a label of other probes in the set. In some cases, each of the at least two sets of at least three probes comprises a reference probe, wherein the reference probe anneals to a second chromosome, and wherein the second chromosome is different from the first chromosome. In some cases, the reference probe in each set anneals to the same sequence of the second chromosome. In some cases, each of the at least two sets of at least three probes comprise three probes that anneal to a different locus of the first chromosome and a reference probe that anneals to a second chromosome, wherein the second chromosome is different from the first chromosome. In some cases, the reference probe in each set comprises the same label. In some cases, the label comprises a dye. In some cases, the dye comprises a fluorescent dye.

In some cases, the at least three loci are located in a region of the chromosome that does not comprise one or more copy number variations. In some cases, each of the at least three loci is located within a span of at least 1 kb of the chromosome. In some cases, each of the at least three loci is located within a span of a chromosome. In some cases, determining the arrangement of the at least three loci comprises use of a computer implemented algorithm.

In some cases, the method further comprises performing next generation sequencing on a sample comprising the first chromosome, thereby generating next generation sequencing data. In some cases, determining the arrangement of the at least three loci comprises inputting the linkage frequencies and next generation sequencing data into a computer implemented algorithm. In some cases, the next generation sequencing data comprises data on one or more chromosome breakpoints. In some cases, the next generation sequencing data is used to select the at least three loci for amplification. In some cases, the next generation sequencing data is used to determine if one or more loci in the sample comprise more than one allele. In some cases, the next generation sequencing data is used to determine if one or more loci in a region with a copy number variation comprise more than one allele.

In some cases, the method further comprises determining if alleles at at least two different loci are located on the same chromosome. In some cases, at least two of the at least three loci differ by a polymorphism. In some cases, determining the arrangement of the at least three loci includes determining a degree of amplification of each of the loci of the chromosome. In some cases, amplifying comprises polymerase chain reaction (PCR). In some cases, PCR comprises digital PCR. In some cases, digital PCR comprises droplet digital PCR. In some cases, a pair of primers is used to amplify each of the plurality of loci.

In some cases, linkage of a locus on the first chromosome and the at least one locus on the second chromosome is 0%. In some cases, determining linkage frequencies comprises enumerating a number of partitions comprising signal from two different probes with different labels. In some cases, determining linkage frequencies comprises enumerating a number of partitions comprising signal from both of two different probes with different labels. In some cases, determining linkage frequencies comprises determining an expected number of partitions that comprise loci that segregate randomly into the same partition. In some cases, determining linkage frequencies comprises measuring a difference between an observed number of partitions that comprise co-localized loci versus an expected number of partitions that comprise co-localized loci due to random Poisson-based distribution of two independently segregating loci.

In some cases, a linkage frequency of two loci that are separated by a smaller distance is greater than a linkage frequency of two loci that are separated by a larger distance. In some cases, a linkage frequency is dependent on a degree of fragmentation of the polynucleotides in the sample. In some cases, a higher degree of fragmentation yields a lower linkage frequency.

In some cases, each set of at least three probes that anneal to the first chromosome consists of three probes with different labels, and the linkage frequencies can be determined among amplified loci to which the three probes anneal. In some cases, the sample is not subjected to a pre-fragmentation step. In some cases, the sample is subjected to a pre-fragmentation step. In some cases, the sample is from a subject with a neurological condition. In some cases, the neurological condition is Alzheimer's disease. In some cases, the neurological condition is autism. In some cases, the neurological condition is schizophrenia.

In some cases, next generation sequencing comprises pyrosequencing. In some cases, next generation sequencing comprises bridge amplification. In some cases, next generation sequencing is used to determine a presence or absence of a copy number variation.

In some cases, the first chromosome comprises one or more copy number variations.

In some cases, the partitioning comprises separating the polynucleotide fragments of the first chromosome such that each partition comprises zero or one polynucleotide fragment of the first chromosome with a locus. In some cases, the partitioning comprises separating the polynucleotide fragments of the first chromosome such that each partition on average comprises about 0.2 copies of a polynucleotide fragment of the first chromosome comprising a locus of the at least three loci. In some cases, the partitioning comprises separating the polynucleotide fragments of the second chromosome such that each partition comprises zero or one polynucleotide fragment of the second chromosome with the at least one locus.

In some cases, the partitioning comprises separating the polynucleotide fragments of the second chromosome such that each partition on average comprises about 0.2 copies of a polynucleotide fragment of the second chromosome comprising a locus of the at least three loci.

In some cases, determining linkage frequencies comprises comparing an abundance of partitions positive for a first locus and a second locus with an abundance of partitions positive for a first locus, second locus, and third locus. In some cases, the abundance of partitions positive for the first locus and second locus is greater than the abundance of partitions positive for the first, second, and third locus, wherein the first locus and second locus are the closest of the three loci in physical distance.

In some cases, the at least three loci comprise loci A, B, and C, and the following populations of partitions are generated: partitions with no loci; partitions with individual loci A, B, or C; partitions with loci A and B; partitions with B and C; and partitions with loci A, B, and C.

In another aspect, a non-transitory computer readable medium is provided having stored thereon sequences of instructions, which, when executed by a computer system, cause the computer system to perform: determining linkage frequencies among at least three amplified loci of a first chromosome, wherein a sample comprising polynucleotide fragments of a first chromosome are obtained; the polynucleotide fragments of the first chromosome are partitioned; at least three loci from the polynucleotide fragments of the first chromosome are amplified; and the at least three amplified loci of the first chromosome are detected with at least three probes, wherein each of the at least three probes comprises a different label; and determining an arrangement of at least three of the loci on the first chromosome based on the linkage frequencies.

In some cases, determining the arrangement of the at least three loci comprises determining a distance between a first locus and a second locus of the at least three loci. In some cases, determining the arrangement of the at least three loci comprises determining a distance between the second locus and a third locus of the at least three loci. In some cases, determining the arrangement of the at least three loci comprises determining a distance between the first locus and the third locus of the at least three loci. In some cases, the distance is a relative distance. In some cases, the distance is determined by comparing the linkage frequencies to a standard. In some cases, the standard is based on linkage frequencies of molecules separated by a known distance. In some cases, determining the arrangement of the at least three loci comprises determining an order of a first locus, second locus, and third locus on the first chromosome.

In some cases, determining linkage frequencies further comprises detecting a plurality of amplified loci of the first chromosome with a second set of at least three probes, wherein a first probe of the first set of probes anneals to a first locus, a second probe of the first set anneals to a second locus, a first probe to the second set anneals to the first locus, and a second probe of the second set of probes anneals to the second locus. In some cases, a third probe of the first set of probes anneals to a third locus, and a third probe of the second set of probes anneals to a fourth locus, wherein the third locus and the fourth locus are not the same.

In some cases, determining linkage frequencies further comprises detecting the at least three amplified loci of the first chromosome with at least two sets of at least three probes, wherein each probe in each set comprises a different label. In some cases, each set of probes comprises probes with the same labels. In some cases, each set of probes comprises at least three probes, wherein each probe in a set comprises a different label.

In some cases, each probe in the at least two sets of probes anneals to a different locus. In some cases, a first set of at least three probes comprises at least one probe that anneals to the same locus as at least one probe in a second set of at least three probes. In some cases, each probe in each set comprises a different label. In some cases, each set of probes comprises the same labels. In some cases, a first set of at least three probes comprises at least two probes that anneal to the same loci as at least two probes in a second set of at least three probes. In some cases, each of at least three sets of probes comprising at least three probes comprises at least one probe that anneals to the same locus as a probe of the other sets of probes. In some cases, each probe that anneals to the same locus comprises the same label.

In some cases, the sample comprises polynucleotide fragments of a second chromosome, wherein the second chromosome is different from the first chromosome.

In some cases, determining linkage frequencies further comprises partitioning the polynucleotide fragments of the second chromosome. In some cases, determining linkage frequencies further comprises amplifying at least one locus of the second chromosome, thereby generating at least one amplified locus of the second chromosome. In some cases, determining linkage frequencies further comprises detecting the at least one amplified locus on the second chromosome with a reference probe, wherein the reference probe is a fourth probe in the set of at least three probes, wherein the reference probe comprises a label different than a label of other probes in the set.

In some cases, each of the at least two sets of at least three probes comprises a reference probe, wherein the reference probe anneals to a second chromosome, and wherein the second chromosome is different from the first chromosome. In some cases, the reference probe in each set anneals to the same sequence of the second chromosome. In some cases, each of the at least two sets of at least three probes comprises three probes that anneal to a different locus of the first chromosome and a reference probe that anneals to a second chromosome, wherein the second chromosome is different from the first chromosome. In some cases, the reference probe in each set comprises the same label. In some cases, the label comprises a dye. In some cases, the dye comprises a fluorescent dye. In some cases, the at least three loci are located in a region of the chromosome that does not comprise one or more copy number variations.

In some cases, each of the at least three loci are located within a span of at least 1 kb of the chromosome. In some cases, each of the at least three loci are located within a span of a chromosome.

In some cases, determining the arrangement of the at least three loci comprises use of a computer implemented algorithm.

In some cases, next generation sequencing is performed on a sample comprising the first chromosome, thereby generating next generation sequencing data. In some cases, determining the arrangement of the at least three loci comprises inputting the linkage frequencies and next generation sequencing data into a computer implemented algorithm. In some cases, the next generation sequencing data comprises data on one or more chromosome breakpoints. In some cases, the next generation sequencing data is used to select the at least three loci for amplification. In some cases, the next generation sequencing data is used to determine if one or more loci in the sample comprise more than one allele. In some cases, the next generation sequencing data is used to determine if one or more loci in a region with a copy number variation comprise more than one allele.

In some cases, it is further determined if alleles at at least two different loci are located on the same chromosome. In some cases, at least two of the at least three loci differ by a polymorphism. In some cases, determining the arrangement of the at least three loci includes determining a degree of amplification of each of the loci of the chromosome.

In some cases, the amplifying comprises polymerase chain reaction (PCR). In some cases, the PCR comprises digital PCR. In some cases, the digital PCR comprises droplet digital PCR. In some cases, a pair of primers is used to amplify each of the plurality of loci. In some cases, linkage of a locus on the first chromosome and the at least one locus on the second chromosome is 0%. In some cases, determining linkage frequencies comprises enumerating a number of partitions comprising signal from two different probes with different labels. In some cases, determining linkage frequencies comprises enumerating a number of partitions comprising signal from both of two different probes with different labels. In some cases, determining linkage frequencies comprises determining an expected number of partitions that comprise loci that segregate randomly into the same partition. In some cases, determining linkage frequencies comprises measuring a difference between an observed number of partitions that comprise co-localized loci versus an expected number of partitions that comprise co-localized loci due to random Poisson-based distribution of two independently segregating loci.

In some cases, a linkage frequency of two loci that are separated by a smaller distance is greater than a linkage frequency of two loci that are separated by a larger distance. In some cases, a linkage frequency is dependent on a degree of fragmentation of the polynucleotides in the sample. In some cases, a higher degree of fragmentation yields a lower linkage frequency. In some cases, each set of at least three probes that anneal to the first chromosome consists of three probes with different labels, and the linkage frequencies are determined among amplified loci to which the three probes anneal.

In some cases, the sample is not subjected to a pre-fragmentation step. In some cases, the sample is subjected to a pre-fragmentation step.

In some cases, the sample is from a subject with a neurological condition. In some cases, the neurological condition is Alzheimer's disease. In some cases, the neurological condition is autism. In some cases, the neurological condition is schizophrenia.

In some cases, the next generation sequencing comprises pyrosequencing. In some cases, the next generation sequencing comprises bridge amplification. In some cases, next generation sequencing is used to determine a presence or absence of a copy number variation.

In some cases, the first chromosome comprises one or more copy number variations.

In some cases, the partitioning comprises separating the polynucleotide fragments of the first chromosome such that each partition comprises zero or one polynucleotide fragment of the first chromosome with a locus. In some cases, the partitioning comprises separating the polynucleotide fragments of the first chromosome such that each partition on average comprises about 0.2 copies of a polynucleotide fragment of the first chromosome comprising a locus of the at least three loci. In some cases, the partitioning comprises separating the polynucleotide fragments of the second chromosome such that each partition comprises zero or one polynucleotide fragment of the second chromosome with the at least one locus. In some cases, the partitioning comprises separating the polynucleotide fragments of the second chromosome such that each partition on average comprises about 0.2 copies of a polynucleotide fragment of the first chromosome comprising a locus of the at least three loci.

In some cases, determining linkage frequencies comprises comparing an abundance of partitions positive for a first locus and a second locus with an abundance of partitions positive for a first locus, second locus, and third locus. In some cases, the abundance of partitions positive for the first locus and second locus is greater than the abundance of partitions positive for the first, second, and third locus, and wherein the first locus and second locus are the closest of the three loci in physical distance.

In some cases, the at least three loci comprise loci A, B, and C, and wherein the following populations of partitions are generated: partitions with no loci; partitions with individual loci A, B, or C; partitions with loci A and B; partitions with B and C; and partitions with loci A, B, and C.

In another aspect, a method for determining a distance between a first locus and second locus on a first polynucleotide is provided, the method comprising a) partitioning a sample comprising the first and second locus into a plurality of partitions; b) determining a number of partitions that comprise the first locus but not the second locus; c) determining a number of partitions that comprise the second locus but not the first locus; d) determining a number of partitions that comprise the first locus and the second locus; e) determining a number of partitions that comprise neither the first locus nor the second locus; f) determining, based on the numbers in steps b-e, a linkage frequency of the first locus and second locus in the sample; and g) based on the linkage frequency, determining a distance between the first locus and second locus on the first polynucleotide.

In some cases, the first polynucleotide is a chromosome.

In some cases, determining distance comprises comparing the linkage frequency of the first locus and second locus to a standard. In some cases, the standard is generated based on a second linkage frequency. In some cases, the second linkage frequency is a linkage frequency of at least two loci separated by a known distance on a second polynucleotide. In some cases, the first polynucleotide and the second polynucleotide are the same. In some cases, the first polynucleotide and the second polynucleotide are different. In some cases, the first polynucleotide and the second polynucleotide are from the same sample. In some cases, the first polynucleotide and the second polynucleotide are from different samples. In some cases, the first polynucleotide and the second polynucleotide are the same chromosome from the same sample. In some cases, the first polynucleotide is a first chromosome and the second polynucleotide is a second chromosome.

In some cases, the standard is a standard curve. In some cases, the standard is an equation. In some cases, the equation is based on linkage frequencies of a plurality of pairs of loci. In some cases, the plurality of pairs of loci are each separated by a known distance. In some cases, distances are known based on sequencing data. In some cases, the plurality of pairs of loci each share a common locus. In some cases, the plurality of pairs of loci are on the same second polynucleotide. In some cases, the first polynucleotide and the second polynucleotide are the same. In some cases, first polynucleotide and the second polynucleotide are different. In some cases, the first polynucleotide and the second polynucleotide are from the same sample. In some cases, the first polynucleotide and the second polynucleotide are from different samples. In some cases, the first polynucleotide and the second polynucleotide are the same chromosome from the same sample. In some cases, the first polynucleotide is a first chromosome and the second polynucleotide is a second chromosome.

In some cases, the first polynucleotide is from a subject with a tri-nucleotide repeat disease. In some cases, the first locus and the second locus flank a region with a tri-nucleotide repeat region. In some cases, the tri-nucleotide repeat region is expanded. In some cases, the tri-nucleotide repeat disease is Fragile X, Huntington's disease, Dentatorubropallidoluysian atrophy, Spinobulbar muscular atrophy, Kennedy disease, Spinocerebellar ataxia, Friedreich's ataxia, or Myotonic dystrophy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features are set forth with particularity in the appended claims. A better understanding of the features and advantages will be obtained by reference to the following detailed description that sets forth illustrative embodiments, in which the principles of the methods and compositions described herein are utilized, and the accompanying drawings of which:

FIG. 1 illustrates an embodiment of a 4-plex linkage assay for mapping genomic rearrangements.

FIG. 2 illustrates a four-dimensional droplet amplitude plot drawn as a two-dimensional figure for a 4-plex linkage assay in which each of the four probes fluoresces in a different channel, shown here as a quadrant.

FIG. 3 illustrates hypothetical results of a hypothetical linkage analysis assay for mapping genomic rearrangements.

FIG. 4 illustrates a chart with hypothetical results for a hypothetical linkage analysis.

FIG. 5 illustrates hypothetical results for a hypothetical linkage analysis of a chromosome with genomic rearrangements.

FIG. 6 illustrates hypothetical results for a hypothetical linkage analysis of a chromosome with genomic rearrangements.

FIG. 7 illustrates a flowchart for estimating the copy number of a target sequence.

FIG. 8 illustrates an example where two target sequences are on a maternal chromosome and an example where one target sequence is on a maternal chromosome and one is on a paternal chromosome.

FIG. 9a illustrates a flowchart for determining the linkage of a target sequence.

FIG. 9b illustrates an alternative workflow for determining the linkage of a target sequence.

FIG. 10 illustrates examples of genetic rearrangements that can be analyzed with a collocation assay.

FIG. 11 is a flowchart listing steps that may be performed in an exemplary method of haplotype analysis using amplification performed in sample partitions, in accordance with aspects of the present disclosure.

FIG. 12 is a schematic view of selected aspects of an exemplary system for performing the method of FIG. 11, in accordance with aspects of present disclosure.

FIG. 13 is a schematic view of exemplary haplotypes that may be created by a pair of SNPs located on the same chromosome type in the genetic material of a subject, in accordance with aspects of the present disclosure.

FIG. 14 is a schematic view of a flowchart illustrating performance of an exemplary version of the method of FIG. 11, with droplets as partitions and with the genetic material from the subject of FIG. 13 being analyzed to distinguish the potential haplotypes presented in FIG. 13, in accordance with aspects of the present disclosure.

FIG. 15 is a graph illustrating an alternative approach to correlating the amplification data of FIG. 14, in accordance with aspects of the present disclosure.

FIG. 16 illustrates a flowchart for predicting fragmentation between two targets.

FIG. 17 illustrates linked and unlinked targets. FIG. 17A illustrates unlinked targets T1 and T2.

FIG. 17B illustrates a mixture of linked T1 and T2 and unlinked T1 and T2. FIG. 17C illustrates different spacings between T1 and T2.

FIGS. 18 and 19 illustrate information that can be considered when selecting a restriction enzyme.

FIGS. 20A and 20B illustrate assay information that can be entered into a database.

FIG. 21 illustrates an example of a workflow for a ddPCR experiment.

FIG. 22 illustrates maximum extension in droplet generation.

FIG. 23 illustrates maximum extension as a function of sample flow rate.

FIG. 24 depicts droplet properties of undigested samples 1-10 and digested samples 11-20.

FIGS. 25A and 25B illustrate haplotyping through collocation.

FIG. 26 is a schematic illustrating sequences recognized by FAM and VIC probes separated by 1K, 10K, or 100K bases.

FIG. 27 illustrates fragments of nucleic acid. T1 and T2 are target sequences. FIG. 27A illustrates a scenario in which T1 and T2 are always on separate nucleic acids (total fragmentation). FIG. 27B illustrates a scenario in which T1 and T2 are always linked on a nucleic acid (no fragmentation). FIG. 27C illustrates a scenario in which T1 and T2 are linked on some nucleic acids and are also on separate nucleic acids (partial fragmentation).

FIG. 28 illustrates a DNA quality assessment.

FIG. 29 illustrates linkage analysis using copied loci with different alleles.

FIG. 30 illustrates another embodiment of a linkage analysis.

FIG. 31 illustrates a linkage analysis.

FIG. 32 illustrates a “mile” marker assay.

FIG. 33 illustrates a percentage of linked molecules on the Y axis as a function of the distance separating the “mile” markers from the anchor sequence on the X-axis.

FIG. 34 illustrates all the genes in the human genome sorted according to their length, as measured from the start codon to the stop codon.

### DETAILED DESCRIPTION

### Overview

Provided herein are methods, compositions, and kits for mapping a chromosomal region. Amplification by, e.g., polymerase chain reaction (PCR), e.g., digital PCR (dPCR), e.g., droplet digital PCR (ddPCR), can be used for the chromosome mapping. In some cases, PCR (e.g., dPCR) and next generation sequencing are used to map a chromosomal region to enable an accurate genome assembly. Digital PCR can be used to determine an arrangement of loci on a chromosome, e.g., a directional order of loci on a chromosome. In some cases, digital PCR can be used to determine a presence or absence of chromosome rearrangements. The presence or absence of chromosome rearrangements can be determined by making a comparison to a reference chromosome. The reference chromosome can have one or more rearrangements; in some cases, the reference chromosome does not have one or more rearrangements. In some cases, presence or absence of chromosome rearrangements is determined without making a comparison to a reference chromosome.

Relative copy number information derived by next generation sequencing can be coupled with long-range information measured by dPCR to generate a chromosome map. For example, next generation sequencing data can provide information on a breakpoint in DNA, and this information can be useful for making a final chromosome assembly. A final chromosome assembly can include a map of a region, distances between different regions, and/or a degree of amplification of each region. This information can be used to help identify disease and methods of treating disease.

In some cases, chromosome mapping can involve one or more of the following techniques: next generation sequencing (including next-generation paired-end sequencing), PCR (e.g., digital PCR), fluorescence in situ hybridization (FISH), microarray-based assay, long-range PCR, Southern blot analysis, comparative genomic hybridization, and karyotyping. For example, next generation sequencing of a sample can suggest that multiple alleles of a copy number variation region are detected. For example, a first gene can have multiple copies per cell, e.g., five copies/cell, and these copies can have a polymorphism (e.g., a SNP) that enables them to be distinguished. The polymorphisms can be used to map where each allele resides in the nucleic acids in the sample (e.g., on the same or different chromosomes).

Linkage of loci on polynucleotides can be used for directional chromosome mapping. Due to fragmentation of polynucleotides in a sample, loci that are more distantly separated on a chromosome can be less likely to be physically linked on a polynucleotide than loci that are less distantly physically separated on the chromosome. This phenomenon can give rise to the ability to generate directional mapping information. For example, in a digital PCR experiment, the frequency of co-location of loci in a partition under dilute conditions can reflect the distance separating the loci on a chromosome. Two loci that are relatively close together on a chromosome can collocate in a single partition more frequently than two loci that are relatively distant to one another on a single chromosome.

The methods described herein can be used on a polynucleotide that is not a chromosome. In some cases, methods described herein are used on an artificial chromosome or synthetic chromosome.

Determining Chromosomal Rearrangements and Directional Chromosome Mapping

A sample comprising a plurality of polynucleotides can be used for chromosome mapping. The plurality of polynucleotides can comprise a plurality of polynucleotides from a first chromosome. The plurality of polynucleotides can comprise a plurality of polynucleotides from a second chromosome. The plurality of polynucleotides can comprise a plurality of polynucleotides from a first chromosome and a second chromosome. The plurality of polynucleotides can be a plurality of polynucleotides from about 1, 2, 3, 4, 5, 6, 7, 8, 9, 10, 11, 12, 13, 14, 15, 16, 17, 18, 19, 20, 21, 22, 23, or 24 chromosomes (e.g., a human chromosome). The plurality of polynucleotides can be a plurality of polynucleotides from at least 1, 2, 3, 4, 5, 6, 7, 8, 9, 10, 11, 12, 13, 14, 15, 16, 17, 18, 19, 20, 21, 22, 23, or 24 chromosomes. The plurality of polynucleotides can be a plurality of polynucleotides from more than 1, 2, 3, 4, 5, 6, 7, 8, 9, 10, 11, 12, 13, 14, 15, 16, 17, 18, 19, 20, 21, 22, 23, or 24 chromosomes. The plurality of polynucleotides can be a plurality of polynucleotides from less than 1, 2, 3, 4, 5, 6, 7, 8, 9, 10, 11, 12, 13, 14, 15, 16, 17, 18, 19, 20, 21, 22, 23, or 24 chromosomes.

Fragmentation of a nucleic acid can separate linked loci. In some cases, a plurality of polynucleotide fragments in a sample can be generated by fragmentation. For example, nucleic acid in a sample can be subjected to a pre-fragmentation step by mechanical shearing, passing the sample through a syringe, sonication, heat treatment (e.g., 30 mins at 90° C.), and/or nuclease treatment (e.g., with DNase, RNase, endonuclease, exonuclease, or restriction enzyme). A sample can be subjected to multiple pre-fragmentation steps. In some cases, a sample is not subjected to a pre-fragmentation step; e.g., in some cases, fragments are generated as a side effect of a purification process. Polynucleotide fragments in a size range can be selected by, e.g., separation by gel electrophoresis and purification, size exclusion chromatography, or dialysis. In some cases, fragmentation of nucleic acids can occur during purification of nucleic acids from a sample. For example, fragmentation of nucleic acids can differ based on whether a magnetic bead-based method or silica-based method is used for preparation of the nucleic acids.

In some cases, polynucleotide fragments of less than 10 Mb, 5 Mb, 1 Mb, 0.5 Mb, 0.1 Mb, 50 kb, 25 kb, 10 kb, 5 kb, or 1 kb are selected. In some cases, polynucleotide fragments of more than 10 Mb, 5 Mb, 1 Mb, 0.5 Mb, 0.1 Mb, 50 kb, 25 kb, 10 kb, 5 kb, or 1 kb are selected. In some cases, polynucleotide fragments of about 10 Mb, 5 Mb, 1 Mb, 0.5 Mb, 0.1 Mb, 50 kb, 25 kb, 10 kb, 5 kb, or 1 kb are selected. In some cases, polynucleotide fragments of at least 10 Mb, 5 Mb, 1 Mb, 0.5 Mb, 0.1 Mb, 50 kb, 25 kb, 10 kb, 5 kb, or 1 kb are selected. In some cases, a polynucleotide is an entire chromosome. Polynucleotide fragments of with an average size of about 10 Mb, 5 Mb, 1 Mb, 0.5 Mb, 0.1 Mb, 50 kb, 25 kb, 10 kb, 5 kb, or 1 kb can be selected. Polynucleotide fragments with a size of about 1 kb to about 10 Mb, about 1 kb to about 1 Mb, about 1 kb to about 0.1 Mb, about 1 kb to about 10 kb, or about 10 kb to about 100 kb can be selected.

A method for determining an arrangement of loci on a first chromosome is provided herein, the method comprising: a) obtaining a sample comprising polynucleotide fragments of the first chromosome; b) partitioning the polynucleotide fragments of the first chromosome; c) amplifying a plurality of loci from the polynucleotide fragments of the first chromosome, thereby generating a plurality of amplified loci of the first chromosome; d) detecting the plurality of amplified loci of the first chromosome with at least three probes, wherein each of the at least three probes comprises a different label; e) determining linkage frequencies of the amplified loci of the first chromosome; and f) based on the linkage frequencies, determining the arrangement of loci on the first chromosome. The arrangement of loci can comprise at least three loci. The arrangement of loci can include the order of loci on a linear nucleic acid and/or the distance between loci on a linear nucleic acid. In some cases, the arrangement of the loci a linear nucleic acid can include a directional ordering of loci on a chromosome. A distance between loci can be a quantitative distance, a semi-quantitative distance, an estimated distance, a calculated distance, an absolute distance, or a relative distance.

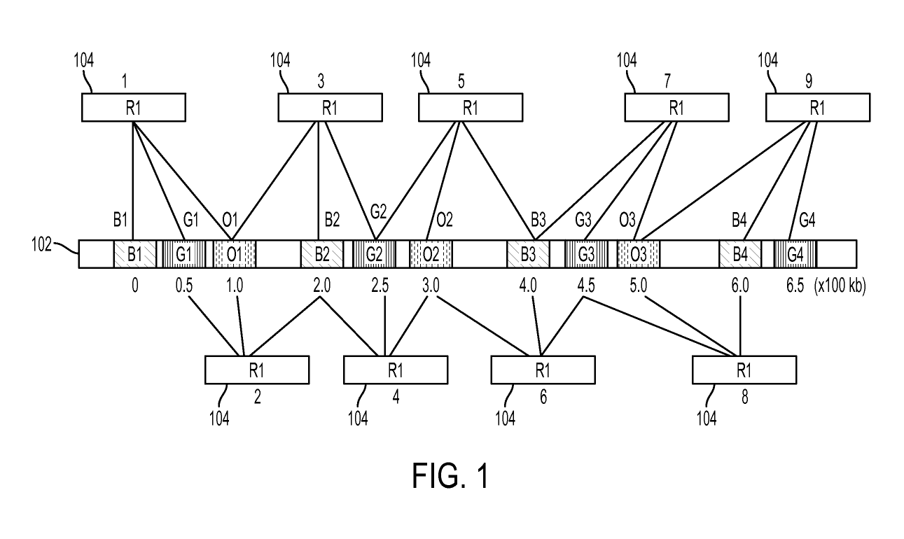

An assay can be performed with a set of probes to determine an arrangement of loci on a chromosome. For example, a set of four probes can be used to perform a 4-plex assay. In some cases, a 4-plex assay is used to generate information on chromosome arrangement, rearrangement, and/or directional mapping information of a chromosome. An example of an assay for determining an arrangement of loci on a first chromosome using a plurality of 4-plex assays is provided in Example 1 and is illustrated in FIG. 1. A 4-plex assay can comprise a set of probes comprising or consisting of four probes. The four probes in a set can have different labels, e.g., different dyes, e.g., different fluorophores. In some cases, a set of probes comprises three probes with three different labels, and these probes anneal to different loci on a first chromosome, and a fourth (reference) probe with another label anneals to a locus on a second chromosome (e.g., control chromosome). In some cases, a first chromosome and second chromosome are different. In some cases, a first chromosome and second chromosome are the same. A plurality of 4-plex assays can be used to map a chromosome (e.g., determine the order of loci on a chromosome and/or determine distance between loci on a chromosome). In some cases, a probe to a second chromosome that is different from the first chromosome can be used to determine if the first chromosome is polysomic or if one or more parts of the first chromosome comprises a copy number variation. For example, if the first chromosome is amplified, the number of partitions with a signal from a first locus on the first chromosome can be more than the number of partitions with a signal from a locus on the second reference chromosome.

FIG. 1 illustrates an example of nine 4-plex assays. A first (“1”) 4-plex assay can comprise four probes: a probe that anneals to B1, a probe that anneals to G1, a probe that anneals to O1, and a probe that anneals to R1. The probes for B1, G1, and O1 can anneal to a first chromosome (102). The probe for R1 can anneal to a second chromosome (104). Each probe in the first 4-plex assay can have a different label (e.g., a dye that fluoresces at a different color: B (blue); G (green); O (orange); and R (red)). The frequency of co-localization of the probes in a digital assay (e.g., dPCR, e.g., ddPCR), can be used to determine a frequency of linkage of loci to which the probes anneal. In this example, under conditions in which nucleic acids comprising loci are diluted, R1 should not frequently co-localize with B1, G1, or O1 because the first chromosome (102) comprising loci B1, G1, and O1 is distinct from (e.g., not physically connected to) the second chromosome (104) comprising locus R1.

When multiple assays are used to analyze a nucleic acid, one or more probes from the different assays can anneal to the same loci. For example, when multiple 4-plex assays are used to analyze nucleic acid, probes from different 4-plex assays can anneal to the same loci. For example, the second 4-plex assay (“2”) in FIG. 1 can comprise four probes: a probe that anneals to G1, a probe that anneals to O1, a probe that anneals to B2, and a probe that anneals to R1. The second 4-plex assay can contain three probes that anneal to the same loci as the probes in the first 4-plex assay (G1, O1, and R1). Two the probes shared between the first 4-plex assay and the second 4-plex assay can anneal to the same loci on the first chromosome (102): G1 and O1. One of the probes shared between the first 4-plex assay and the second 4-plex assay, R1, can anneal to the second chromosome (104). The third 4-plex assay (“3”) can comprise a probe to O1, a probe to B2, and a probe to G2, and a probe to R1. Three of the probes of the third 4-plex assay, O1, B2, and R1, can anneal to the same sequences as three of probes in the second 4-plex assay. Two of the probes in the third 4-plex assay, O1 and B2, can anneal to the same loci on the first chromosome as two of the probes in the second 4-plex assay. One of the probes in the third 4-plex assay, R1, can anneal to the same locus as a probe in the second 4-plex assay.

Probes that anneal to the same loci can have the same sequence. In some cases, probes that anneal to the same loci can have a different sequence. For example, two distinct probes that anneal to the same locus can have different lengths or anneal to different regions of the locus.

Linkage frequencies of loci in one or more 4-plex assays can be used to determine the order of loci on a chromosome and distances between one or more loci on a chromosome or a nucleic acid fragment.

A plurality of 4-plex assays can be used to analyze a chromosome, e.g., a plurality of 4-plex assays can be used to determine an order of loci on a chromosome and/or distances between loci on chromosome. For example, about 2, 5, 10, 25, 50, 100, 250, 500, 1000, 2500, 5000, 10,000, 25,000, 50,000, or 100,000 4-plex assays can be used to analyze a chromosome. About 2 to about 10, about 10 to about 25, about 25 to about 100, about 100 to about 250, about 250 to about 1000, about 1000 to about 2500, about 2500 to about 10,000, or about 10,000 to about 100,000 4-plex assays can be performed to analyze a chromosome. In some cases, more than 2, 5, 10, 25, 50, 100, 250, 500, 1000, 2500, 5000, 10,000, 25,000, 50,000, or 100,000 4-plex assays can be used to analyze a chromosome.

A distance between two loci on a chromosome that can be determined using a 4-plex assay can be about 2, 5, 10, 25, 50, 100, 250, 500, 1000, 2500, 5000, 10,000, 25,000, 50,000, 75,000, or 100,000, 250,000, 500,000, 750,000, or 1,000,000 bp or bases. A distance between two loci on a chromosome that can be determined using a 4-plex assay can be less than 2, 5, 10, 25, 50, 100, 250, 500, 1000, 2500, 5000, 10,000, 25,000, 50,000, 75,000, or 100,000, 250,000, 500,000, 750,000, or 1,000,000 bp or bases. A distance between loci on a chromosome that can be determined using a 4-plex assay can be about more than 2, 5, 10, 25, 50, 100, 250, 500, 1000, 2500, 5000, 10,000, 25,000, 50,000, 75,000, 100,000, 250,000, 500,000, 750,000, or 1,000,000 bp or bases. A distance between loci on a chromosome that can be determined using a 4-plex assay can be about 2 to about 10 bases or bp, about 10 to about 100 bases or bp, about 100 to about 1000 bases or bp, about 1000 to about 10,000 bases or bp, about 10,000 to about 100,000 bases or bp, or about 100,000 to about 1,000,000 bases or bp. A distance between loci can be determined using a standard.

The directional order of multiple loci can be determined on a chromosome using a method, composition, and/or kit described herein. For example, the number of loci that can be ordered on a chromosome using a method described herein can be about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 500, 1000, 5000, 10,000, 50,000, 100,000, 500,000, or 1,000,000. The number of loci that can be ordered on a chromosome using a method, composition, or kit described herein can be less than 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 500, 1000, 5000, 10,000, 50,000, 100,000, 500,000, or 1,000,000. The number of loci that can be ordered on a chromosome using a method, composition, or kit described herein can be more than 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 500, 1000, 5000, 10,000, 50,000, 100,000, 500,000, or 1,000,000. The number of loci that can be ordered on a chromosome using a method, composition, or kit described herein can be about 2 to about 10, about 10 to about 25, about 25 to about 50, about 25 to about 100, about 100 to about 500, about 100 to about 1000, about 1000 to about 5000, about 1000 to about 10,000, about 10,000 to about 100,000, or about 100,000 to about 1,000,000.

In some cases, a 3-plex assay is used to generate information on chromosome arrangement, rearrangement, and/or directional mapping information of a chromosome. A 3-plex assay can comprise a set of probes comprising or consisting of three probes with three different labels, e.g., different dyes, e.g., different fluorophores. In some cases, a set of probes comprises three probes with three different labels, and the probes anneal to different loci on a first chromosome. In some cases, none of the probes in a set anneals to a second chromosome, where the second chromosome is different from the first chromosome. A plurality of 3-plex assays can be used to map a chromosome. A 3-plex assay can lack a probe that anneals to a control chromosome; for example, all three probes in a 3-plex assay can anneal to a first chromosome. A plurality of 3-plex assays can be used to analyze a chromosome. For example, a first (“1”) 3-plex assay can comprise three probes: a probe that anneals to B1, a probe that anneals to G1, and a probe that anneals to O1 (see FIG. 1 for exemplary order of loci). The probes for B1, G1, and O1 can anneal to a first chromosome. Each probe in the first 3-plex assay can have a different label (e.g., a dye that fluoresces at a different color: B (blue); G (green); O (orange); and R (red)). The frequency of co-localization of the probes in a digital assay (e.g., dPCR, e.g., ddPCR), can be used to determine the frequency of linkage of loci to which the probes anneal.

When multiple 3-plex assays are used to analyze nucleic acids, probes from different 3-plex assays can anneal to the same loci. For example, a second 3-plex assay (“2”) can comprises three probes: a probe that anneals to G1, a probe that anneals to O1, and a probe that anneals to B2 (see FIG. 1 for exemplary order of loci). Two probes in the first 3-plex assay and in the second 3-plex assay can anneal to the same loci on the first chromosome: G1 and O1. A third 3-plex assay (“3”) can comprise a probe to O1, a probe to B2, and a probe to G2 (see FIG. 1 for exemplary order of loci). Two of the probes of the third 3-plex assay, O1, and B2, can anneal to the same sequences as two of probes in the second 3-plex assay. Two of the probes in a third 3-plex assay, O1 and B2, can anneal to the same loci on the first chromosome as two of the probes in the second 3-plex assay (see FIG. 1 for exemplary order of loci).

Linkage frequencies of loci in one or more 3-plex assays can be used to determine the order of loci on a chromosome and distances between one or more loci on a chromosome. The distances between loci that can be determined using one or more 3-plex assays can be the same as distances that can be determined using 4-plex assays described above. Distances can be determined by comparing a linkage frequency to a standard.

In some cases, a 2-plex assay is used to generate information on chromosome arrangement, rearrangement, and/or directional mapping information of a chromosome. A 2-plex assay can comprise a set of probes comprising or consisting of two probes with two different labels, e.g., different dyes, e.g., different fluorophores. In some cases, a set of probes comprises two probes with two different labels, and the probes anneal to different loci on a first chromosome. In some cases, none of the probes in a set anneals to a second chromosome, wherein the second chromosome is different from the first chromosome. A plurality of 2-plex assays can be used to map a chromosome (e.g., more than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 2-plex assays). When a plurality of 2-plex assays is used to analyze a chromosome, each assay can comprise one probe that is the same as a probe in another 2-plex assay. A label on a probe in at least two different 2-plex assays can be the same in the at least two different 2-plex assays. Examples of distances between loci that can be determined using one or more 2-plex assays can be the distances that can be determined using a 4-plex assay.

In some cases, an assay used to analyze a chromosome is a 2-plex (comprising or consisting of 2 probes), 3-plex (comprising of consisting of 3 probes), 4-plex assay (comprising or consisting of 4 probes), 5-plex assay (comprising or consisting of 5 probes), 6-plex (comprising or consisting of 6 probes), 7-plex (comprising or consisting of 7 probes), 8-plex (comprising or consisting of 8 probes), 9-plex (comprising or consisting of 9 probes), or 10-plex assay (comprising or consisting of 10 probes), in which one probe comprising a first label anneals to a locus on a control chromosome, and probes with other labels anneal to different loci on a target chromosome.

A plurality of 2-plex, 3-plex, 4-plex, 5-plex, 6-plex, 7-plex, 8-plex, 9-plex, or 10-plex assays can be used to map a chromosome. A combination of sets of probes can be used to directionally order loci on a chromosome. The number of probes among sets used to directionally order loci on a chromosome can be different (e.g., a first set of probes can comprise 3 probes, and a second set of probes can comprise 4 probes). The number of probes between sets of probes or between assays that can anneal to the same loci can be n-1, n-2, n-3, n-4, n-5, n-6, n-7, n-8, n-9, n-10, wherein n is the total number of probes in each set of probes or assay. For example, the number of probes between two 4-plex assays or two sets of 4 probes that can anneal to the same loci can be 3 (4-1), 2 (4-2), 1 (4-3), or 0 (4-4). The number of probes between two 3-plex assays or two sets of 3 probes that can anneal to the same loci can be 2 (3-1), 1 (3-2), or 0 (3-3). The number of probes between two 5-plex assays or two set of 5 probes that can anneal to the same loci can be 4 (5-1), 3 (5-2), 2 (5-3), 1 (5-4), or 0 (5-5).

In some cases, a set of probes does not comprise a probe that anneals to a control chromosome. A control chromosome can be the same as the chromosome being mapped, and a control probe can anneal at least 100, 1000, 10,000, 100,000, or 1,000,000 bases away from loci of interest.

In some cases, a probe is in solution. In some cases, a probe is attached is a solid support, e.g., a bead or chip.

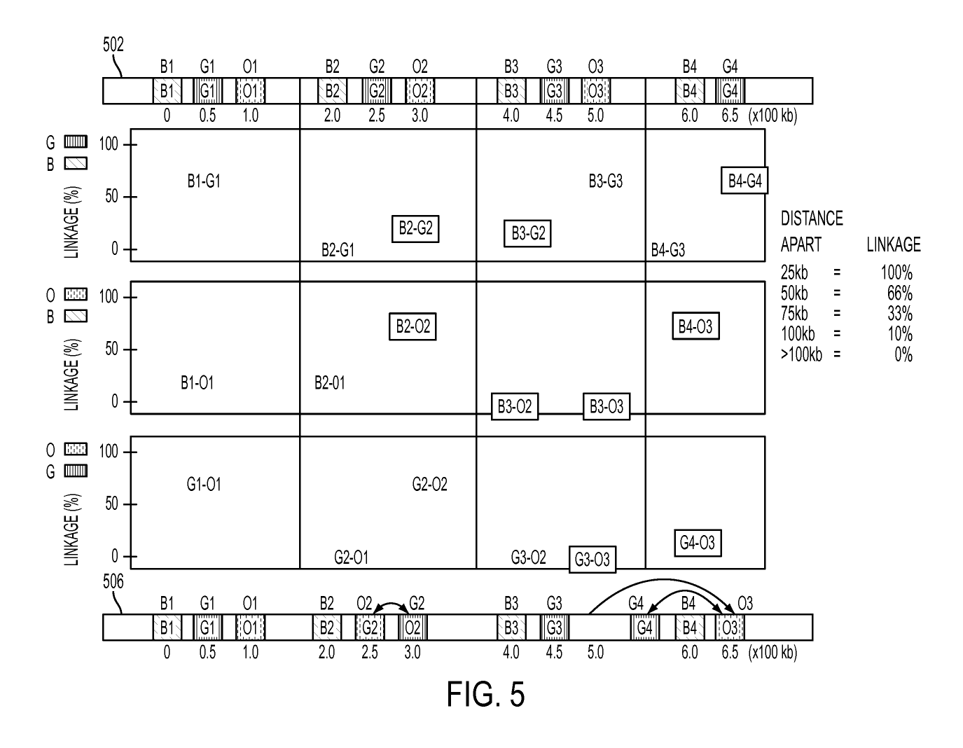

An assay described herein can be used to determine an arrangement of loci on a chromosome. Once determined, the information can serve as a reference chromosome for determining an arrangement of loci another chromosome. FIG. 5 illustrates an example of arrangement of loci on a reference chromosome (502) and arrangement of loci on a second chromosome (506). FIG. 5 illustrates that certain loci on the second chromosome are rearranged relative to loci on the reference chromosome. A reference chromosome with an arrangement of loci can be derived from a database, e.g., genome database.

In some cases, probes in a set that anneal to a first chromosome can each anneal to loci on a stretch of nucleic acid sequence on the first chromosome that is about 0.001, 0.0025, 0.005, 0.0075, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.6, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Mb. In some cases, probes in a plurality of sets of probes that anneal to a first chromosome can each anneal to loci on a stretch of nucleic acid sequence on the first chromosome that is about 0.001, 0.0025, 0.005, 0.0075, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.6, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Mb.

Probes in a set that anneal to a first chromosome can each anneal to loci on a stretch of nucleic acid sequence on the first chromosome that is more than 0.001, 0.0025, 0.005, 0.0075, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.6, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Mb. In some cases, probes in a plurality of sets of probes that anneal to a first chromosome can each anneal to loci on a stretch of nucleic acid sequence on the first chromosome that is more than 0.001, 0.0025, 0.005, 0.0075, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.6, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Mb. In some cases, probes in a plurality of sets of probes that anneal to a first chromosome can each anneal to loci on a stretch of nucleic acid sequence on the first chromosome that is the entire length of the first chromosome.

Probes in a set that anneal to a first chromosome can each anneal to loci on a stretch of nucleic acid sequence on the first chromosome that is less than 0.001, 0.0025, 0.005, 0.0075, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.6, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Mb. In some cases, probes in a plurality of sets of probes that anneal to a first chromosome can each anneal to loci on a stretch of nucleic acid sequence on the first chromosome that is less than 0.001, 0.0025, 0.005, 0.0075, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.6, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Mb.

Probes in a set that anneal to a first chromosome can each anneal to loci on a stretch of nucleic acid sequence on the first chromosome that is at least 0.001, 0.0025, 0.005, 0.0075, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.6, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Mb. In some cases, probes in a plurality of sets of probes that anneal to a first chromosome can each anneal to loci on a stretch of nucleic acid sequence on the first chromosome that is at least 0.001, 0.0025, 0.005, 0.0075, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.6, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Mb.

In some cases, the stretch of nucleic acid on the first chromosome to which probes in a set can anneal is about 0.01 to about 1 MB, about 0.01 to about 0.1 MB, about 0.01 to about 0.05 MB, about 50 kb to about 100 kb, about 50 kb to about 200 kb, or about 50 kb to about 500 kb. In some cases, probes in a plurality of sets of probes that anneal to a first chromosome can each anneal to loci on a stretch of nucleic acid sequence on the first chromosome that is about 0.01 to about 1 MB, about 0.01 to about 0.1 MB, about 0.01 to about 0.05 MB, about 50 kb to about 100 kb, about 50 kb to about 200 kb, or about 50 kb to about 500 kb.

In some cases, a plurality of sets of 4 probes is used for directional mapping of a chromosome. Each probe in a set can comprise a different label, while each set of probes can comprise the same labels. In some cases, the labels among sets of probes are different. In some cases, each probe in a set anneals to a different locus.

In some cases, the number of sets of probes used to determine an arrangement of loci on a chromosome, and/or map a chromosome is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000.

In some cases, the number of sets of probes used to determine an arrangement of loci on a chromosome, and/or map a chromosome is more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000.

In some cases, the number of sets of probes used to determine an arrangement of loci on a chromosome, and/or map a chromosome is less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000.

In some cases, the number of sets of probes used to determine an arrangement of loci on a chromosome, and/or map a chromosome is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000.

In some cases, the number of sets of probes used to determine an arrangement of loci on a chromosome, and/or map a chromosome is about 1 to about 1000, about 1 to about 100, about 1 to about 10, about 5 to about 500, about 5 to about 100, about 10 to about 100, about 2 to about 20, about 5 to about 100, about 10 to about 100, about 10 to about 50, about 5 to about 50, or about 5 to about 25.

In some cases, the arrangement of loci on about 1, 2, 3, 4, 5, 6, 7, 8, 9, 10, 11, 12, 13, 14, 15, 16, 17, 18, 19, 20, 21, 22, 23, or 24 chromosomes is determined and/or mapped. In some cases, the arrangement of loci on more than 1, 2, 3, 4, 5, 6, 7, 8, 9, 10, 11, 12, 13, 14, 15, 16, 17, 18, 19, 20, 21, 22, 23, or 24 chromosomes is determined and/or mapped. In some cases, the arrangement of loci on less than 1, 2, 3, 4, 5, 6, 7, 8, 9, 10, 11, 12, 13, 14, 15, 16, 17, 18, 19, 20, 21, 22, 23, or 24 chromosomes is determined and/or mapped. In some cases, the arrangement of loci on at least 1, 2, 3, 4, 5, 6, 7, 8, 9, 10, 11, 12, 13, 14, 15, 16, 17, 18, 19, 20, 21, 22, 23, or 24 chromosomes is determined and/or mapped.

Individual probes among sets of probes can be identical and/or can anneal to the same sequence. For example, a first set of three or four probes can comprise at least two probes that anneal to the same loci as at least two probes in a second set of three or four probes. In some cases, a first set of three or four probes can comprise at least three probes that anneal to the same loci as at least three probes in a second set of three or four probes. In some cases, one of the identical probes (or probes that anneals to the same sequence) among sets of probes anneals to a control chromosome. In some cases, two probes among a set of probes each anneal to the same loci on a target chromosome. In some cases, one probe among a set of probes anneals to the same locus on a target chromosome. In some cases, two probes among a set of probes each anneal to the same loci on a target chromosome, and one probe among the set of probes anneals to the same locus on a control chromosome. In some cases, probes among sets of probes that anneal to the same locus comprise the same label.

Amplification can be used to detect target loci (e.g., detection of loci with probes). In some cases, loci from about 1, 2, 3, 4, 5, 6, 7, 8, 9, 10, 11, 12, 13, 14, 15, 16, 17, 18, 19, 20, 21, 22, 23, or 24 chromosomes are amplified. In some cases, loci from more than 1, 2, 3, 4, 5, 6, 7, 8, 9, 10, 11, 12, 13, 14, 15, 16, 17, 18, 19, 20, 21, 22, 23, or 24 chromosomes are amplified. In some cases, loci from less than 1, 2, 3, 4, 5, 6, 7, 8, 9, 10, 11, 12, 13, 14, 15, 16, 17, 18, 19, 20, 21, 22, 23, or 24 chromosomes are amplified. In some cases, loci from at least 1, 2, 3, 4, 5, 6, 7, 8, 9, 10, 11, 12, 13, 14, 15, 16, 17, 18, 19, 20, 21, 22, 23, or 24 chromosomes are amplified.

In some cases, a plurality of loci from a first chromosome is amplified, and a single locus from a second chromosome is amplified. In some cases, a plurality of loci from a first chromosome is amplified, and a plurality of loci from a second chromosome is amplified. In some case, at least one amplified locus on the second chromosome is detected with a fourth probe in a set, wherein the fourth probe comprises a label different than a label of the at least three probes in the set.

In some cases, a pair of primers is used to amplify each of the plurality of loci. The amplified loci can be detected by annealing probes to the loci. The amplifying can comprise polymerase chain reaction (PCR), the PCR can be digital PCR, and the digital PCR can be droplet digital PCR. The amplification can comprise any amplification technique described herein.

In a digital PCR assay, nucleic acids can be partitioned, and the partitioning can comprise separating polynucleotide fragments of a first chromosome such that each partition comprises on average about 0, 0.1, 0.2, 0.3, 0.4, 0.5, 0.6, 0.7, 0.8, 0.9, 1, 1.1, 1.2, 1.3, 1.4, 1.5, 2, 2.5, 3, 3.5, 4, 4.5, or 5 polynucleotide fragments of the first chromosome with at least one target locus; the partitioning can also comprise separating polynucleotide fragments of a second chromosome such that each partition comprises on average 0, 0.1, 0.2, 0.3, 0.4, 0.5, 0.6, 0.7, 0.8, 0.9, 1, 1.1, 1.2, 1.3, 1.4, 1.5, 2, 2.5, 3, 3.5, 4, 4.5, or 5 polynucleotide fragments of the second chromosome with at least one target locus. In some cases, each partition comprises 0 or 1 polynucleotide fragments from a first chromosome comprising at least one target locus. In some cases, each partition comprises 0 or 1 polynucleotide fragments from a second chromosome comprising at least one target locus. In some cases, a partition comprises an entire chromosome. In some cases, a partition comprises an entire genome.

Partitioning can comprise separately haploid genome equivalents. In some cases, each partition on average comprises about 0, 0.2, 0.3, 0.4, 0.5, 0.6, 0.7, 0.8, 0.9, 1, 1.1, 1.2, 1.3, 1.4, 1.5, 2, 2.5, 3, 3.5, 4, 4.5, or 5 haploid genome equivalents. Each partition can have 0, 0.2, 0.3, 0.4, 0.5, 0.6, 0.7, 0.8, 0.9, 1, 1.1, 1.2, 1.3, 1.4, 1.5, 2, 2.5, 3, 3.5, 4, 4.5, or 5 haploid genome equivalents. In some cases, each partition comprises 0 or 1 haploid genome equivalents.

In some cases, the methods described herein, e.g., chromosome mapping, do not involve nucleic acid amplification. Polynucleotides can be detected with a probe without amplification.

A label on a probe can be any label described herein. In some cases, a label on a probe comprises a dye. The dye can be any dye described herein. For example, the dye can comprise a fluorescent dye. The fluorescent dye can comprise FAM™, VIC™, or NED™ (Life Technologies).

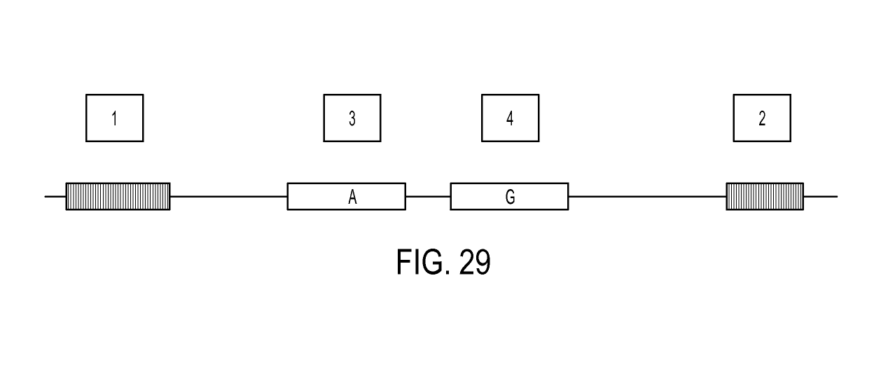

In some cases, the loci that are amplified and/or detected are located in a region of the chromosome that does not comprise one or more copy number variations. In some cases, the loci that are amplified and/or detected are located in a region of a chromosome that does comprise one or more copy number variations. A first chromosome can comprise one or more copy number variations. Next generation sequencing can be used to determine a presence or absence of a copy number variation. In some cases, small nucleotide polymorphisms can be used to distinguish between different copies of chromosomes in a region with copy number variation. In some cases, one or more alleles (e.g., SNPs) can be assayed to determine which copy of an amplified section is closer or farther from an anchor point (loci). If amplified (copied) segments are identical, the order of the segments may or may not be determined. For example, FIG. 29 illustrates the architecture of a chromosome mapped in a linkage analysis using different alleles of a copied locus. Boxes shaded with either vertical (1) or horizontal lines (2) are unique sequences. Empty rectangles (3 and 4) represent identical copies of a gene, which, e.g., could be 1 Mb in length. The only difference between loci (3) and (4) is that that loci (3) has a mutation changing a base to an ‘A’ allele, while loci (4) has a ‘G’ allele, the wild-type allele in this example. The presence of this SNP can allow for these two copies to be appropriately mapped. For example, linkage analysis (e.g., a 3-plex reaction) can be performed across two wells; a second well can be used for confirmation. The first well can have assays (primers and probes) for detecting the following loci: 1, 3 (A allele), and 4(G allele)); the second well can have assays (primers and probes for detecting locus 3 (A allele), locus 4 (G allele), and locus 2). Based on the relative abundance of partitions with locus 1, 3 (A allele) to the abundance of partitions with locus 1, locus 3 (A allele), and locus 4 (G allele), it can be determined that locus 3 (A allele) is closer to locus 1 than locus 4 (G allele) is to locus 1. Likewise, based on the abundance of partitions (e.g., droplets) comprising signal from locus 4 (G allele) and locus 2 relative to the abundance of partitions with locus 3 (A allele), locus 4 (G allele), and locus 2 signal, it can be determined that locus 4 (G allele) is closer to locus 2 than locus 3 (A allele) is to locus 2.

As described above, linkage analysis for mapping purposes can be accomplished by comparing an abundance of double positive partitions (e.g., droplets) relative to triple positive partitions (e.g., droplets).

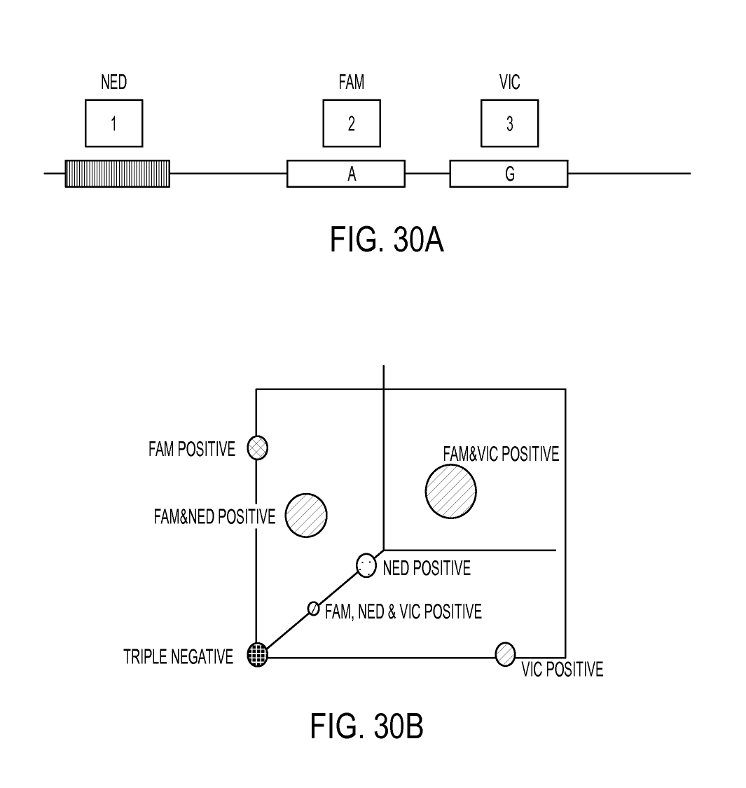

FIG. 30A illustrates a portion of a chromosome, wherein locus 1 is a unique locus, and loci 2 and 3 are copies that differ by a single SNP-loci 2 has an “A” and loci 3 has a “G”. FIG. 30B illustrates a 3-dimensional fluorescence amplitude plot for the portion of the chromosome illustrated in FIG. 30A. Assuming random shearing occurs, single positive partitions (e.g., droplets) are expected each for locus 1, locus 2 (A allele), and locus 3 (G allele). The sample can be analyzed at a very low DNA load, such that the possibility of double positive partitions (e.g., droplets) from random colocalization of individual fragments is very low. In this case, because locus 2 (A allele) is positioned between locus 1 and locus 3 (G allele), double positive locus 1/locus 3 (G allele) partitions (e.g., droplets) would not be expected to be observed without locus 2 (A allele) also being present in the partition, unless these two targets (locus 1 and locus 3) randomly co-localized to the same partition (e.g., droplet). In the plot in FIG. 30B, the size of each circle represents the number of partitions (e.g., droplets) in the cluster. NED, FAM, and VIC are labels on probes for loci 1, 2 (A allele), and 3 (G allele). Here, because the FAM−NED cluster is larger than the FAM, NED, VIC cluster, the target detected by VIC is outside (5′ or 3′) to the region of DNA that contains the targets for the probes labeled with FAM and NED. The FAM and VIC cluster is larger than the FAM and NED cluster. This result suggests that locus 3 (VIC) is closer to locus 2 (FAM) than locus 2 (FAM) is to locus 1 (NED). One or more additional tri-plex assays can be run to learn if locus 3 (G allele—VIC) is 5′ or 3′ of loci 2.

Determining linkage frequencies can comprises measuring a difference between an observed number of partitions (e.g., droplets) that comprise co-localized loci versus an expected number of partitions that comprise co-localized loci due to random Poisson-based distribution of two independently segregating loci. In some cases, the determining arrangement of loci and/or mapping loci includes determining distances between loci of the first chromosome. Determining an arrangement of loci and/or mapping loci can include determining a degree of amplification of each of the loci of the chromosome. Determining an arrangement of loci on a chromosome can comprise determining a distance between loci on a chromosome and determining an order of loci on a chromosome. In some cases, determining an arrangement of loci on a chromosome can comprise determining distance between loci on a chromosome, determining an order of loci on a chromosome, and determining a degree of amplification of loci on a chromosome.

In some cases, linkage of a locus on a first chromosome and at least one locus on the second chromosome is 0%. In some cases, the first chromosome and second chromosome are different. In some cases, linkage of a locus on a first chromosome and a locus on a second chromosome is greater than 0%. In some cases, the first chromosome and second chromosome are the same. Determining linkage frequencies can comprise enumerating a number of partitions comprising signal from two different probes with different labels. The linkage frequency of two loci that are separated by a smaller distance can be greater than the linkage frequency of two loci that are separated by a larger distance. The linkage frequency can be dependent on a degree of fragmentation of the polynucleotides in the sample. For example, a higher degree of fragmentation can yield a lower linkage frequency.

In some cases, determining the proximity of three loci to one another is achieved by directly comparing the abundance of double-positive versus triple-positive droplets, where the double-positive cluster contains more partitions (e.g., droplets) than the triple-positive cluster, and the loci amplified in the double-positive cluster are the two loci of the three screened that are closest to one another.

In some cases, for a 3-plex assay, where an amount of DNA used is low enough that a random distribution of two independent loci into the same partition (e.g., droplet) is not expected, then one expects to see only negative partitions (e.g., droplets), single-positive partitions (e.g., droplets) for each of the three loci (A, B, and C), two double-positive cluster (A/B and B/C), and a single triple-positive cluster (A, B, and C). In this case, there can be two double-positives (A/B and B/C), rather than three double-positive clusters (no A/C), because in this example loading occurs in a regime where the other double positive cluster (A/C) should only occur through random distribution of fragmented copies. In some cases, the following populations of partitions are generated: partitions with no loci; partitions with individual loci A, B, or C; partitions with loci A and B; partitions with B and C; and partitions with loci A, B, and C. In some cases, fragmented loci can randomly co-localize to the same partition. For example, if locus A and locus C are separated by a large distance, and locus A is on one fragment of nucleic acid, and locus C is on a separate nucleic acid fragment, on occasion, a nucleic acid fragment with locus A and a nucleic acid fragment with locus C can co-localize to the same partition.

In some cases, each set of at least three probes that anneal to loci on a first chromosome consists of three probes with different labels, and the linkage frequencies are determined among amplified loci to which the three probes anneal.

Linkage frequency can be determined by comparing the total number of partitions with a first locus and/or a second locus relative to the number of partitions in which the first and second locus are colocalized. An algorithm can be used to generate a chromosome map based on the linkage frequencies of multiple loci.

Chromosome mapping can be illustrated with an ideogram, (or ideograms). In some cases, chromosome mapping makes use of the International System for Cytogenetic Nomenclature (ISCN). In the ISCN scheme, numbering for a chromosome can begin at a centromere. A chromosome can have a short arm (p, petite arm) and a long arm (q, queue arm). Each arm of a chromosome can be divided into regions, and numbers assigned to each region can get larger as the distance from the centromere to the telomere increase.

Also provided herein are methods, compositions, and kits for analyzing nucleic acid sequence, e.g., by digital partitioning. Digital portioning can be used for linkage analysis. Also, provided herein are methods, compositions, and kits for estimating the number of copies of a target nucleic acid sequence in a sample, e.g., a genome. Also provided herein are methods, compositions, and kits for determining linkage or haplotype information of one or more target sequences in a sample, e.g., a genome. Haplotyping information can be information regarding whether or not multiple copies of one target sequence are on a single or multiple chromosomes. Using the concept of collocation of different targets within the same partition, it can be practical to infer phase, i.e., whether a particular allele of one mutation or a SNP is physically linked to an allele of another mutation or a SNP. Methods, compositions, and kits are also provided herein for determining an extent of fragmentation or degradation of a nucleic acid sample (e.g., a genomic DNA sample, RNA sample, mRNA sample, DNA sample, cRNA sample, cDNA sample, miRNA sample, siRNA sample), by, e.g., digitally analyzing collocating signals. In another aspect, methods are provided herein for finding inversions, translocations, and deletions.

Copy Number Variation Estimation

In some cases, copy number variation information is used in chromosome mapping. Digital PCR can be used to analyze copy number variations. In some cases, digital analysis (e.g., dPCR) of copy number of a target sequence can underestimate the number of copies of a target nucleic acid sequence in a sample if multiple copies of the target nucleic acid sequence are on the same polynucleotide in a sample. For example, in a digital PCR assay that has multiple compartments (e.g., partitions, spatially isolated regions), nucleic acids in a sample can be partitioned such that each compartment receives on average about 0, 1, 2, or several target polynucleotides. Each partition can have, on average, less than 5, 4, 3, 2, or 1 copies of a target nucleic acid per partition (e.g., droplet). In some cases, at least 0, 1, 2, 3, 4, 5, 6, 7, 8, 9, 10, 20, 30, 40, 50, 60, 70, 80, 90, 100, 125, 150, 175, or 200 partitions (e.g., droplets) have zero copies of a target nucleic acid. The number of compartments that contain a polynucleotide can be enumerated. However, if two copies of a target nucleic acid sequence are on a single polynucleotide a compartment containing that polynucleotide can be counted as having only one target sequence.

Methods provided herein can determine the relative position of target sequences. For example, a target sequence can be present in an organism or cell in multiple copies, e.g., about 2, 3, 4, 5, 6, 7, 8, 9, 10, 20, 30, 40, 50, 60, 70, 80, 90, 100, 125, 150, 175, 200, 500, 1000, 5000, 10,000, 50,000, or 100,000 copies, or more than 2, 3, 4, 5, 6, 7, 8, 9, 10, 20, 30, 40, 50, 60, 70, 80, 90, 100, 125, 150, 175, 200, 500, 1000, 5000, 10,000, 50,000, or 100,000 copies. Target sequences can each have a sequence difference relative to each other; for example, five target sequences can be present in a cell or organism, and each target sequence can differ by a polymorphism. Different target sequences can vary from each other by at least 1, 5, 10, 100, or 1000 bases or by of sequence. Methods provided herein can be used to determine the relative positions of the different target sequences in a nucleic acid sample (e.g., whether the targets are on the same or different chromosomes).

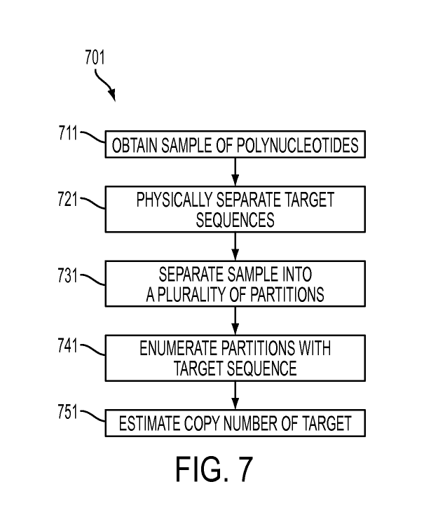

In some cases, to determine copy number variation, target nucleic acids sequences can be physically separated. Methods provided herein can avoid underestimating copy numbers of a target sequence due to the presence of multiple copies of the target sequence on a single polynucleotide. FIG. 7 illustrates an overview of an embodiment of a method of copy number estimation (701); this figure and the other figures provided in this disclosure are for illustrative purposes only and are not intended to limit methods described herein. The steps in FIG. 7 can be performed in any suitable order and combination and can be united with any other steps of the present disclosure. A first sample of polynucleotides is obtained (711); the first sample can be, e.g., a genomic DNA sample. The target nucleic acid sequences in the first sample can be physically separated (e.g., by contacting the first sample with one or more restriction enzymes) (721). The first sample can be separated into a plurality of partitions (731). The number of partitions with the target sequence can be enumerated (741). The copy number of the target can then be estimated (751).

The target nucleic acids can be identical; or, in other cases, the target nucleic acids can be different. In some cases, the target nucleic acids are located within the same gene. In some cases, the target nucleic acids are each located in a different copy (identical or near identical copy) of a gene. In still other cases, the target sequences are located within introns, or in a region between genes. Sometimes, one target sequence is located in a gene; and the second target sequence is located outside of the gene. In some cases, a target sequence is located within an exon.

In some cases, a genome comprises one target sequence. In some cases, a genome comprises two or more target sequences. When a genome comprises two or more target sequences, the target sequences can be about, or more than 50%, 55%, 60%, 65%, 70%, 75%, 80%, 85%, 90%, 95%, or 100% identical.

Physically separating two target sequences can comprise physically separating the target sequences by cleaving a specific site on the nucleic acid sequence. In some cases, the physically separating target nucleic acid sequences can comprise contacting the first sample with one or more restriction enzymes. Physically separating the target nucleic acid sequences can comprise digesting a polynucleotide at a site located between the target nucleic acid sequences. In some cases, the target nucleic acid sequences are each located within a gene. In some cases, the site that is targeted for digestion is located between the two genes. In some cases, the site selected for digestion is located in a gene; and, in some cases, the gene is the same gene as the gene which contains the target sequences. In other cases, the site selected for digestion is located in a different gene from that of the target sequence. In some cases, a target sequence and the site targeted for digestion are located in the same gene; and the target sequence is located upstream of the site targeted for digestion. In other cases, a target sequence and the site targeted for digestion are located in the same gene; but the target sequence is located downstream of the site targeted for digestion. In some cases, target nucleic acids can be separated by treatment of a nucleic acid sample with one or more restriction enzymes. In some cases, target nucleic acids can be separated by shearing. In some cases, target nucleic acids can be separated by sonication.

Following the physical separation step (e.g., digesting with one or more restriction enzymes), the sample can be partitioned into multiple partitions. Each of the plurality of partitions can comprise about 0, 1, 2 or several target polynucleotides. Each partition can have, on average, less than 5, 4, 3, 2, or 1 copies of a target nucleic acid per partition (e.g., droplet). In some cases, at least 0, 1, 2, 3, 4, 5, 6, 7, 8, 9, 10, 20, 30, 40, 50, 60, 70, 80, 90, 100, 125, 150, 175, or 200 droplets have zero copies of a target nucleic acid.

Target nucleic acid can be amplified in the partitions. In some cases, the amplification comprises use of one or more TaqMan probes.

A method can further comprise the step of enumerating the number of partitions comprising a reference nucleic acid sequence. A reference nucleic acid sequence can be known to be present in a certain number of copies per genome and can be used to estimate the number of genome copies of a target nucleic acid sequence in a sample. Estimating the copy number can comprise comparing the number of partitions comprising the target sequence to the number of partitions comprising the reference nucleic acid sequence. A CNV estimate can be determined by a ratio of the concentration of target nucleic acid sequence to a reference sequence.

A method can further comprise the step of analyzing a second sample, wherein the second sample and the first sample are derived from the same sample (e.g., a nucleic acid sample is split to the first sample and the second sample). A method can further comprise not contacting the second sample with one or more restriction enzymes. In some cases, a method further comprises separating the second sample into a plurality of partitions. A method can further comprise enumerating the number of partitions of the second sample that comprise the target sequence. A method can further comprise enumerating the number of partitions of the second sample that comprise a reference sequence. A method can comprise estimating the copy number of the target sequence in the second sample. Estimating the copy number of the target sequence in the second sample can comprise comparing the number of partitions from the second sample with the target sequence and the number of partitions from the second sample with the reference sequence.

The copy number of the target sequence from the first sample and the copy number of the target sequence in the second sample can be compared to determine whether the copy number of the target sequence in the second sample was underestimated. The degree to which the copy number was underestimated may be indicative of whether interrogated copies were all on one chromosome or if at least one copy was on one homologous chromosome and at least one copy was on the other homologous chromosome. Values closer to one per diploid genome may indicate the first case, while values closer to two may indicate the second case.

Additional methods of determining copy number differences by amplification are described, e.g., in U.S. Patent Application Publication No. 20100203538. Methods for determining copy number variation are described in U.S. Pat. No. 6,180,349 and Taylor et al. (2008) PLoS One 3(9): e3179.

When employing methods described herein, a variety of features can be considered:

Sample preparation: Properties of nucleic acids to be considered can include secondary structure, amplicon length, and degree of fragmentation. An assay can be performed to determine the degree of fragmentation of a nucleic acid sample. If the degree of fragmentation of a nucleic acid sample is too high, the sample can be discarded from an analysis. Steps can be taken to eliminate secondary structure of nucleic acids in a sample. Secondary structure of a nucleic acid can be modulated, for example, by regulating the temperature of a sample or by adding an additive to a sample. It can be determined whether a potential amplicon is too large to be efficiently amplified. In one embodiment, a Bioanalyzer is used to assess nucleic acid (e.g., DNA) fragmentation. In another embodiment, size exclusion chromatography is used to assess nucleic acid (e.g., DNA) fragmentation.

Dynamic range: Increasing the number of partitions or spatially isolated regions can increase the dynamic range of a method. Template nucleic acid can be diluted into a dynamic range.

Accuracy: If a homogenous sample is used, CNV values can be expected to fall on integer values (self-referencing). Drop-out amplification can cause inaccurate concentration measurements and, therefore, inaccurate CNV determinations. Additives (e.g., DMSO) can be added in GC-rich assays.

Multiplexing: An experiment can be multiplexed. For example, two colors can be used in the methods provided herein: FAM: BHQ and NFQ-MGB assays; VIC: NFQ-MGB, TAMRA. HEX: BHQ, 5′ and 3′ labeling can be used, and an internal labeled dye can be used. In some cases, the number of colors used in the methods provided herein is greater than two, e.g., greater than 3, 4, 5, 6, 7, 8, 9, or 10 colors.

Precision: Increased precision can be accomplished in several ways. In some cases, increasing the number of droplets in a dPCR experiment can increase the ability to resolve small differences in concentration between target and reference nucleic acids. Software can enable “metawell” analysis by pooling replicates from individual wells. In some cases, the methods provided herein enable detection of a difference in copy number that is less than 30%, 20%, 15%, 14%, 13%, 12%, 11%, 10%, 9%, 8%, 7%, 6%, 5%, 4%, 3%, 2%, or 1%.

Assay landscape: Target gene assays described herein can be combined with commercially available or custom designed target gene assays.

Copy number variations described herein can involve the loss or gain of nucleic acid sequence. Copy number variations can be inherited or can be caused by a de novo mutation. A CNV can be in one or more different classes. See, e.g. Redon et al. (2006) Global variation in copy number in the human genome. Nature 444 pp. 444-454. A CNV can result from a simple de novo deletion, from a simple de novo duplication, or from both a deletion and duplication. A CNV can result from combinations of multi-allelic variants. A CNV can be a complex CNV with de novo gain. A CNV can include about, or more than 0, 1, 2, 3, 4, 5, 6, 7, 8, 9, or 10 contiguous genes. A CNV can include about 1 to about 10, about 1 to about 5, about 1 to about 4, about 1 to about 3, about 1 to about 2, about 0 to about 10, about 0 to about 5, or about 0 to about 2 contiguous genes. A copy number variation can involve a gain or a loss of about, or more than, 100, 500, 1000, 2000, 3000, 4000, 5000, 6000, 7000, 8000, 9000, 10,000, 20,000, 30,000, 40,000, 50,000, 60,000, 70,000, 80,000, 90,000, 100,000, 200,000, 500,000, 750,000, 1 million, 5 million, or 10 million base pairs. In some cases, a copy number variation can involve the gain or loss of about 1,000 to about 10,000,000, about 10,000 to about 10,000,000, about 100,000 to about 10,000,000, about 1,000 to about 100,000, or about 1,000 to about 10,000 base-pairs of nucleic acid sequence. A copy number variation can be a deletion, insertion, or duplication of nucleic acid sequence. In some cases, a copy number variation can be a tandem duplication.

In some cases, CNV haplotypes can be estimated from fluorescent signals generated by real-time PCR or ddPCR of partitioned samples. Before the late stages of a real-time PCR or ddPCR experiment, when reagents can become limiting, a partition with a higher copy number of a target sequence can have a higher signal than a partition with a lower copy number of the target sequence. A sample (e.g., a subsample of a sample used in a linkage experiment) can be partitioned, and PCR can be performed on the partitions (e.g., droplets). The mean fluorescence intensity of partitions can be determined as they undergo exponential amplification for a target and/or reference nucleic acid sequence. The mean intensity can correspond to the number of starting copies of the target. If multiple targets are linked along a single polynucleotide strand, the intensity in the partition (e.g., droplet) that captures this strand may be higher than that of a partition (e.g., droplet) that captures a strand with only a single copy of the target. Excess presence of positive droplets with higher mean amplitudes can suggest the presence of a haplotype with multiple CNV copies. Conversely, presence of positive droplets with only low mean amplitudes can suggest that only haplotypes with single CNV copies are present in the sample. The number of cycles used to estimate CNV can be optimized based on the size of the partitions and the amount of reagent in the partitions. For example, smaller partitions with lower amounts of reagent can require fewer amplification cycles than larger partitions that would be expected to have higher amounts of reagent.

The methods described herein can be used to analyze target copies that are near each other on a polynucleotide, e.g., less than 10, 9, 8, 7, 6, 5, 4, 5, 2, 1, 0.7 0.5, 0.3, 0.2, 0.1, 0.05, or 0.01 megabases apart; or that are very near each other on a polynucleotide, e.g., less than 10, 9, 8, 7, 6, 5, 4, 3, 2, or 1 kilobase apart. In some cases, a method provided herein is useful for analyzing target copies that are very close to each other on the polynucleotide, e.g., within about 1, 10, 20, 30, 40, 50, 60, 70, 80, 90, 100, 200, 300, 400, 500, 600, 700, 800, 900, or 950 base pairs (bp's) apart. In some cases, the method is useful for analyzing target copies that are separated by zero (0) base pairs. In some cases, the method can be applied to identical, near identical, and completely different targets.

Additional embodiments of methods for estimating the copy number of one or more target sequences are described herein. In some cases, next generation sequencing (or massively parallel sequencing) is used to determine copy number variation (see e.g., Duan J, Zhang J-G, Deng H-W, Wang Y-P (2013) Comparative Studies of Copy Number Variation Detection Methods for Next-Generation Sequencing Technologies. PLoS ONE 8(3): e59128. doi:10.1371/journal.pone.0059128),

Determining Linkage of Target Sequences

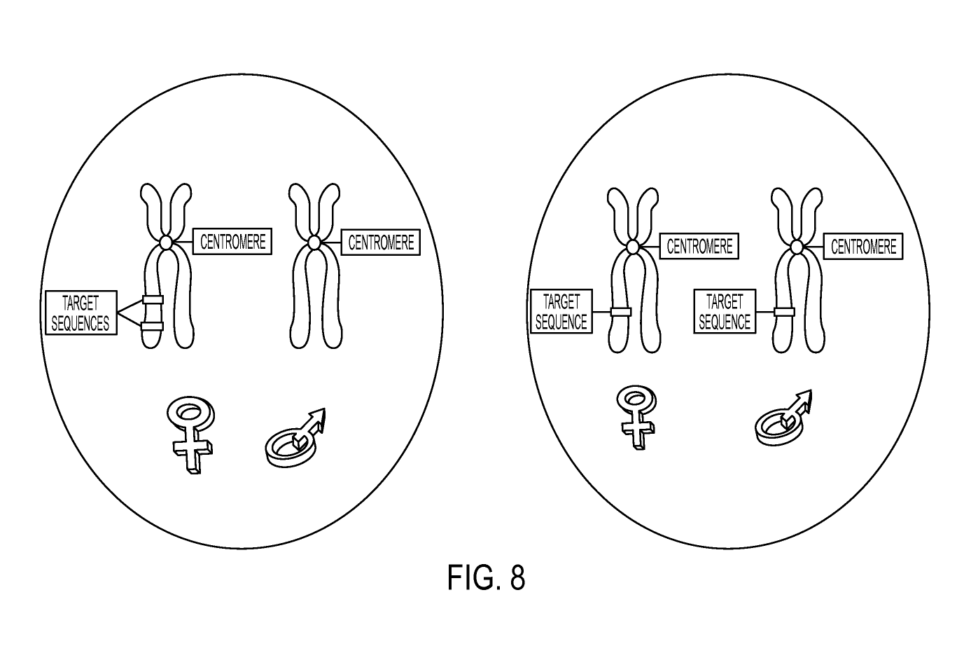

In some cases, chromosome mapping makes use of information concerning the linkage of two or more loci (target sequences). Methods described herein can indicate whether two or more target sequences are linked on a polynucleotide (e.g., the methods can be used to determine the linkage of target sequences). In one embodiment, a method is provided comprising physically separating target sequence copies (e.g., by using one or more restriction enzymes) so that the copies can be assorted independently into partitions for a digital readout, and using a readout of undigested DNA together with a readout from digested DNA to estimate how the target copies are linked. For example, methods described herein can be used to determine if the target sequences are present on the same chromosome or if they are on different chromosomes (see e.g., FIG. 8). FIG. 8 illustrates a nucleus (left) in which a maternal chromosome comprises two copies of a target sequence, but the corresponding paternal chromosome comprises no copies; in the nucleus on the right, a maternal chromosome and the corresponding paternal chromosome each comprise one copy of the target.

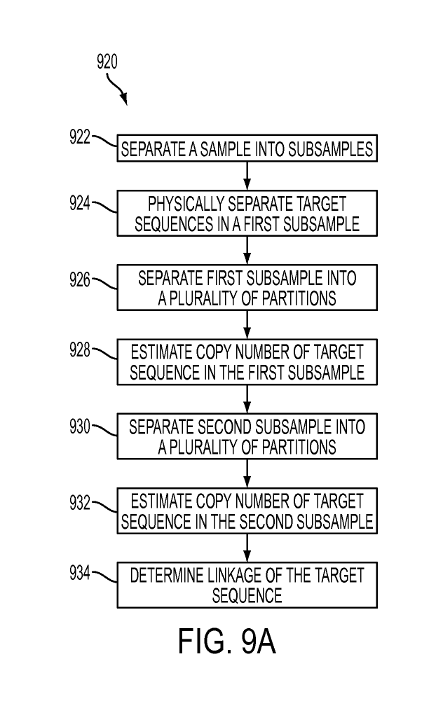

FIG. 9a illustrates a workflow of an embodiment of a method, without being restricted to any order of the steps. In one aspect, a method (920) is provided comprising a) separating a sample comprising a plurality of polynucleotides into at least two subsamples (922); b) physically separating physically linked target sequences in a first subsample (924); c) separating the first subsample into a first set of a plurality of partitions (926); d) estimating the copy number of a target sequence in the first subsample (928); e) separating a second subsample into a second set of a plurality of partitions (930); f) estimating the copy number of the target sequence in the second subsample (932); g) comparing the estimated copy number of the target sequence in the first subsample to the estimated copy number of the target sequence in the second subsample to determine the haplotypes of the target sequence in the sample (934).

Physically separating physically linked target sequences in the first subsample can comprise contacting the first subsample with one or more restriction enzymes. Contacting the sample comprising polynucleotides with one or more restriction enzymes can comprise digesting nucleic acid sequence between at least two target nucleic acid sequences. In some cases, physically linked target nucleic acids can be separated by contacting a nucleic acid sample with one or more restriction enzymes. In some cases, physically linked target nucleic acids can be separated by shearing. In some cases, physically linked target nucleic acids can be separated by sonication.

Each of the plurality of partitions of a first and second subsample comprise about 0, 1, 2 or several target polynucleotides. Each partition can have, on average, less than 5, 4, 3, 2, or 1 copies of a target nucleic acid per partition (e.g., droplet). In some cases, at least 0, 1, 2, 3, 4, 5, 6, 7, 8, 9, 10, 20, 30, 40, 50, 60, 70, 80, 90, 100, 125, 150, 175, or 200 partitions (e.g., droplets) have zero copies of a target nucleic acid.

Target Sequences can be Amplified in Partitions.

Estimating the copy number of a target sequence in a first subsample comprises enumerating the number of partitions of the first subsample comprising the target sequence. Estimating the copy number of the target sequence in the first subsample can comprise enumerating the number of partitions of the first subsample comprising a reference nucleic acid sequence. Estimating the copy number of the target sequence in the first subsample can comprise comparing the number of partitions of the first subsample comprising the target sequence to the number of partitions comprising the reference nucleic acid sequence in the first subsample.

In some cases, the second subsample is not contacted with one or more restriction enzymes. Estimating the copy number of the target sequence in the second subsample can comprise enumerating the number of partitions of the second subsample that comprise the target sequence. Estimating the copy number of the target sequence in the second subsample can comprise enumerating the number of partitions of the second subsample that comprise a reference sequence. Estimating the copy number of the target sequence in the second subsample can comprise comparing the number of partitions from the second subsample with the target sequence and the number of partitions from the second subsample with the reference sequence. The reference sequence for the first and second subsample can be the same sequence or a different sequence.

Determining haplotypes of the target sequence can comprise comparing the estimated copy number of the target sequence in the first subsample to the estimated copy number of the target sequence in the second subsample. Haplotypes can comprise two copies of the target sequence on a single polynucleotide and no copies on the homologous polynucleotide. Haplotyping can comprise one copy of a target sequence on a first polynucleotide and a second copy of the target sequence on a second (possibly homologous) polynucleotide.

In some cases, the greater the difference between copy numbers in the first subsample and the second subsample, the more likely it is that one of the chromosomes does not carry a copy of the target.

FIG. 9b illustrates a workflow of another embodiment of a method, without being restricted to any order of the steps. A method (936) is provided comprising, a) obtaining a sample of polynucleotides (938) and dividing a plurality of polynucleotides into at least two subsamples (940); b) pre-amplifying target sequence in the first subsample with short cycle PCR (942); c) separating the first subsample into a first set of a plurality of partitions (944); d) estimating the copy number of a target sequence in the first subsample (946); e) taking a second subsample that has not been pre-amplified (948) into a second set of a plurality of partitions (950); f) estimating the copy number of the target sequence in the second subsample (952); g) comparing the estimated copy number of the target sequence in the first subsample to the estimated copy number of the target sequence in the second subsample to determine the linkage of the target sequence in the sample (954). See e.g., U.S. Patent Application Publication No. 20120322058, which is incorporated by reference for all purposes.

In some cases, the preamplification used to separate targets is Specific Target Amplification (STA) (Qin et al. (2008) Nucleic Acids Research 36 e16), which can entail performing a short pre-amplification step to generate separate unlinked amplicons for the target nucleic acids.

Pre-amplifying target sequence in the first subsample can comprise contacting the first subsample with a reaction mixture comprising DNA polymerase, nucleotides, and primers specific to the target sequence and amplifying the target sequence for a limited number of cycles. Optionally, the method also comprises using primers for a reference sequence and, optionally, amplifying the reference sequence for a limited number of cycles. In some embodiments, the number for the number of cycles can range from about 4 to about 25 cycles. In some cases, the number of cycles is less than 25, 24, 23, 22, 21, 20, 19, 18, 17, 16, 15, 14, 13, 12, 11, 10, 9, 8, 7, 6, 5, or 4 cycles. The number of cycles may vary depending on the droplet size and the quantity of available reagents. For example, few cycles can be used for partitions (e.g., droplets) that are of smaller size.

The pre-amplified first subsample can be partitioned into multiple partitions, each partition comprising on average less than one target polynucleotide. Each partition can have, on average, less than 5, 4, 3, 2, or 1 copies of a target nucleic acid per partition (e.g., droplet). In some cases, at least 0, 1, 2, 3, 4, 5, 6, 7, 8, 9, 10, 20, 30, 40, 50, 60, 70, 80, 90, 100, 125, 150, 175, or 200 partitions (e.g., droplets) have zero copies of a target nucleic acid.

Estimating the copy number of the target sequence in the first subsample can comprise enumerating the number of partitions of the first subsample comprising a reference nucleic acid sequence. Estimating the copy number of the target sequence in the first subsample can comprise comparing the number of partitions of the first subsample comprising the target sequence to the number of partitions comprising the reference nucleic acid sequence in the first subsample.

In some cases, the second subsample is not subjected to a pre-amplification step. The second subsample can be partitioned into multiple partitions, each partition containing on average about 0, 1, 2, or several target polynucleotides. Each partition can have, on average, less than 5, 4, 3, 2, or 1 copies of a target nucleic acid per partition (e.g., droplet). In some cases, at least 0, 1, 2, 3, 4, 5, 6, 7, 8, 9, 10, 20, 30, 40, 50, 60, 70, 80, 90, 100, 125, 150, 175, or 200 partitions (e.g., droplets) have zero copies of a target nucleic acid. Estimating the copy number of the target sequence in the second subsample can comprise enumerating the number of partitions of the second subsample that comprise the target sequence. Estimating the copy number of the target sequence in the second subsample can comprise enumerating the number of partitions of the second subsample that comprise a reference sequence. Estimating the copy number of the target sequence in the second subsample can comprise comparing the number of partitions from the second subsample with the target sequence and the number of partitions from the second subsample with the reference sequence. The reference sequence for the first and second subsample can be the same sequence or a different sequence.

Determining haplotypes of the target sequence can comprise comparing the estimated copy number of the target sequence in the first subsample to the estimated copy number of the target sequence in the second subsample. The haplotypes can comprise two copies of the target sequence on a single polynucleotide and no copies on the homologous polynucleotide. The haplotypes can comprise one copy of a target sequence on a first polynucleotide and a second copy of the target sequence on a second (possibly homologous) polynucleotide.

In some cases, the greater the difference between copy numbers in the first subsample and the second subsample, the more likely it is that one of the chromosomes does not carry a copy of the target.

In yet another aspect, this disclosure provides a method of identifying a plurality of target nucleic acids as being present on the same polynucleotide comprising, a. separating a sample comprising a plurality of polynucleotides into at least two subsamples, wherein the polynucleotides comprise a first and second target nucleic acid; b. contacting the first subsample with an agent capable of physically separating the first target nucleic acid from the second target nucleic acid if they are present on the same polynucleotide; c. following step b, separating the first subsample into a first set of partitions; d. determining the number of partitions in the first set of partitions that comprise the target nucleic acid; e. separating a second subsample into a second set of partitions; f. determining the number of partitions in the second set of partitions that comprise a target nucleic acid; and g. comparing the value obtained in step d with the value obtained in step f to determine the whether the first and second target nucleic acid are present within the same polynucleotide.

The sample can be of sufficiently high molecular weight so that if a pair of targets is on the same chromosome, they can be mostly linked in solution as well. If the nucleic acid (e.g., DNA) in a sample is completely unfragmented, the readout can be 0, 1, or 2 copies of the target (integers). However, because nucleic acid (e.g., DNA) can be partially degraded, copy numbers can span non-integer values, as well as numbers greater than 2. Another step can be taken to assess nucleic acid fragmentation of a sample, e.g., by using gels, a Bioanalyzer, size exclusion chromatography, or a digital PCR co-location method (milepost assay). If a nucleic acid sample is found to be overly fragmented, this decreases the likelihood information can be gleaned about linkage.

This approach can be used to determine smaller copy number states, e.g., 2, 3, 4.

A method of linkage determination of a target nucleic acid sequence is provided herein making use of probes with two different labels (e.g., VIC and FAM) to detect the same target sequence. For example, a nucleic acid sequence can be separated into a plurality of spatially-isolated partitions, the target sequence can be amplified in the partitions, and the two different probes can be used to detect the target sequence. The nucleic acid sample can be partitioned such that on average about 0, 1, 2, or several target polynucleotides are in each partition. Each partition can have, on average, less than 5, 4, 3, 2, or 1 copies of a target nucleic acid per partition (e.g., droplet). In some cases, at least 0, 1, 2, 3, 4, 5, 6, 7, 8, 9, 10, 20, 30, 40, 50, 60, 70, 80, 90, 100, 125, 150, 175, or 200 partitions (e.g., droplets) have zero copies of a target nucleic acid.

If a partition comprises two targets linked on a polynucleotide, the partition can have signal for a first probe (e.g., VIC only (VIC/VIC)), a second probe (e.g., FAM only (FAM/FAM)), or for both probes (e.g., VIC and FAM). Overabundance of partitions with VIC and FAM signal in a partition compared to what is expected from random dispersion of first and second probe targets can indicate that the sample contained polynucleotides that have at least two targets linked on a polynucleotide. Lack of overabundance of partitions with both signals (e.g., VIC and FAM) can indicate that two target nucleic acid sequences are not linked in a sample.

Determining Distances Between Loci

Provided herein are methods for determining a distance between loci on a polynucleotide. A method for determining a distance between a first locus and second locus on a first polynucleotide is provided herein, the method comprising a) partitioning a sample comprising the first and second locus into a plurality of partitions; b) determining a number of partitions that comprise the first locus but not the second locus; c) determining a number of partitions that comprise the second locus but not the first locus; d) determining a number of partitions that comprise the first locus and the second locus; e) determining a number of partitions that comprise neither the first locus nor the second locus; f) determining, based on the numbers in steps b-e, a linkage frequency of the first locus and second locus in the sample; and g) based on the linkage frequency, determining a distance between the first locus and second locus on the first polynucleotide. In some cases, a number of partitions that comprise the first locus but not the second locus is determined and used to determine a linkage frequency between the loci. In some cases, a number of partitions that comprise the second locus but not the first locus is determined and is used to determine a linkage frequency between the loci. In some cases, a number of partitions that comprise the first locus and the second locus is determined and is used to determine a linkage frequency between the loci. In some cases, a number of partitions that comprise neither the first locus nor the second locus is determined. In some cases, only one, two, or three of steps b), c), d), and e) are performed and used to determine a linkage frequency between a first locus and second locus.

The first polynucleotide can be a chromosome, e.g., a human chromosome. Determining distance can comprise comparing the linkage frequency of the first locus and second locus to a standard. The standard can be generated based on a second linkage frequency. The second linkage frequency can be a linkage frequency of at least two loci separated by a known distance on a second polynucleotide.

In some cases, the first polynucleotide and the second polynucleotide are the same (e.g., the same chromosome from the same sample, or the same chromosome (e.g., chromosome 1) from different samples, etc.). In some cases, the first polynucleotide and the second polynucleotide are different (e.g., the first polynucleotide is chromosome 1 from a human sample, and the second polynucleotide is chromosome 2 from the same or different human sample, etc.). In some cases, the first polynucleotide and the second polynucleotide are from the same sample (e.g., the first polynucleotide is chromosome 1 from a sample, and the second polynucleotide is chromosome 2 from the same subject; or the first polynucleotide and the second polynucleotide are chromosome 1 from the same sample, etc.). In some cases, the first polynucleotide and the second polynucleotide are from different samples. In some cases, the first polynucleotide and the second polynucleotide are the same chromosome from the same sample. In some cases, the first polynucleotide is a first chromosome and the second polynucleotide is a second chromosome. In some cases, the first polynucleotide and the second polynucleotide are from samples from different subjects. In some cases, the first polynucleotide and the second polynucleotide are from the same sample from the same subject. In some cases, the first polynucleotide and the second polynucleotide are from different samples from the same subject (e.g., samples taken before and after a subject is administered a treatment).

The standard can be a standard curve. In some cases, the standard is an equation. A standard curve can be a fit of data for linkage frequencies between a plurality of loci and known distances between the loci of each pair. In some cases, the relationship between linkage frequencies between a plurality of loci and known distances between the loci of each pair is linear; in some cases, the relationship is exponential. The equation can be based on linkage frequencies of a plurality of pairs of loci. The plurality of pairs of loci can each be separated by a known distance. The distances can be known based on sequencing data. The plurality of pairs of loci can each share a common locus, e.g., an anchor locus. In some cases, the plurality of pairs of loci is on the same second polynucleotide. In some cases, the first polynucleotide and the second polynucleotide are the same. In some cases, the first polynucleotide and the second polynucleotide are different. In some cases, the first polynucleotide and the second polynucleotide are from the same sample. The first polynucleotide and the second polynucleotide can be from different samples. The first polynucleotide and the second polynucleotide can be the same chromosome from the same sample. The first polynucleotide can be a first chromosome and the second polynucleotide can be a second chromosome. The distance between loci can be an estimated distance or a calculated distance.

In some cases, the methods described herein are used to measure a distance between a first locus and a second locus in a polynucleotide from a subject with a tri-nucleotide repeat disease. In some cases, the first locus and the second locus flank a region with a tri-nucleotide repeat region. In some cases, the first locus and second locus are selected based on results from a sequencing technique, e.g., next generation sequencing. In some cases, the first locus and second locus are selected based on analysis of a reference chromosome or genome. In some cases, the first locus and/or second locus are located with less than 10,000, 1,000, 500, 250, 100, 50, 25, 10, 5, or 2 bases or base pairs of a 5′ end or 3′ end of a tri-nucleotide repeat region. In some cases, the tri-nucleotide repeat region is expanded. In some cases, the tri-nucleotide repeat region comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000 tri-nucleotide repeats. In some cases, the tri-nucleotide region disease is Fragile X, Huntington's disease, Dentatorubropallidoluysian atrophy, Spinobulbar muscular atrophy, Kennedy disease, Spinocerebellar ataxia, Friedreich's ataxia, Myotonic dystrophy. A tri-nucleotide repeat disease can be a polyglutamine (PolyQ) disease, e.g., Dentatorubropallidoluysian atrophy (DRPLA). Huntington's disease (HD), spinocerebellar ataxia Type 1 (SCA1), spinocerebellar ataxia Type 2 (SCA2), Spinocerebellar ataxia Type 3 (SCA3 or Machado-Joseph disease), Spinocerebellar ataxia Type 6 (SCA6), Spinocerebellar ataxia Type 7 (SCAT), or spinocerebellar ataxia 17 (SCA17). In some cases, a tri-nucleotide repeat disease is a non-polyglutamine disease, e.g., Fragile X syndrome (FRAXA), Fragile X-associated tremor/ataxia syndrome (FXTAS), Fragile XE mental retardation (FRAXE), Friedreich's ataxia (FRDA). Myotonic strophe (DM). Spinocerebellar ataxia Type 8) SCA8, or spinocerebellar ataxia Type 12 (SCA12). In some cases, a disease status is determined by based on a distance determined between a first locus and a second locus that flank a tri-nucleotide repeat region.

Collocalization

Sample partitioning and the ability to analyze multiple targets in a partition can allow detection of targets that are spatially clustered together in the sample. This spatial clustering analysis can be done by assessing whether the number of partitions with a particular combination of targets is in statistical excess compared to what would be expected if the targets were randomly distributed in the partitions. The extent of overabundance of such partitions can be used to estimate the concentration of the combination of targets.

For example, one can measure two targets: A and B using a digital PCR (e.g., ddPCR). For example, there would be four types of droplets: droplets negative for both targets, droplets positive for A, droplets positive for B and droplets positive for both. Under random distribution the number of double positive droplets should be close to (total number of droplets)*(fraction of droplets with at least B)*(fraction of droplets with at least A). If the number of double positive droplets significantly exceeds the expectation, an inference can be made that the two targets are in proximity to each other in the sample. This result can mean that target A and B are physically linked by virtue of, e.g., being on the same polynucleotide, that they are part of the same protein/nucleic acid complex, that they are part of the same exosome, or that they are part of the same cell.

The presence of a particular target in a partition can be assessed by using a fluorophore specific to that target as part of a probe-based TaqMan assay scheme. For example, when measuring two targets A and B, one can use a probe labeled with FAM for A and a probe labeled with VIC for B. Different targets can be assessed with the same fluorophore or intercalating dye using endpoint fluorescence to distinguish partitions containing A from those containing B from those containing A and B.

Sometimes, random distribution of two loci on different polynucleotide fragments into the same partition does not occur.

Rearrangements

Two assays (e.g., amplicons) can be constructed that are normally far apart from each other on a polynucleotide (e.g., two genes separated by millions of by on a chromosome). One assay is on one channel (e.g., probe labeled with FAM), the other on another channel (e.g., probe labeled with VIC). In a digital amplification method, e.g., dPCR or ddPCR, normally, co-localization in the same partition (e.g., droplet) should not be observed above the baseline statistical expectation. If colocalization of FAM and VIC signal occurs (e.g., as measured a linkage analysis described herein), this can be an indication that the two loci were brought in the vicinity of each other on the genome by a rearrangement. This result can indicate an inversion or a translocation depending on where the loci are normally located in the genome. The assays can also be multiplexed on the same channel if their endpoint fluorescences are distinct enough. More than two assays can be multiplexed to catch multiple inversion/translocation events or to account for the fact that a given translocation may present with different breakpoints.

Detection of rearrangements can be used for diagnosing and prognosing a variety of conditions, including cancer and fetal defects. Detection of rearrangements can be used to select one or more therapeutic treatments for a subject. For example, detection of translocation t(9;22)(q34.1;q11.2) can lead to generation of a BCR-ABL fusion protein, associated with chronic myelogenous leukemia (CML). CML patients that express BCR-ABL can be treated with imatinib (Gleevec).

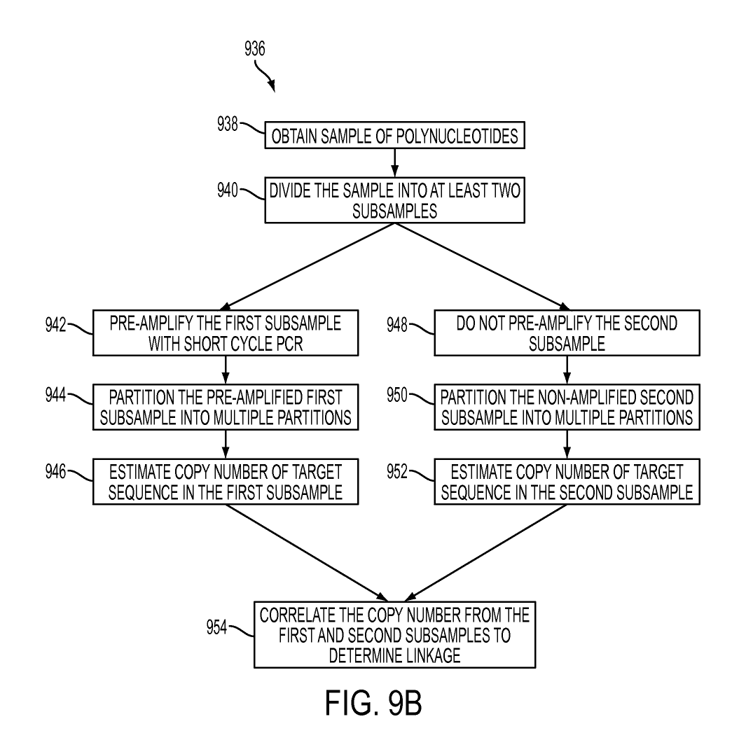

Rearrangements that can be detected with methods described here include, e.g., inversions, translocations, duplications, or deletions (see e.g., FIG. 10).

In some cases, a genome can comprise one or more rearrangements, and next-generation sequencing, digital PCR, and/or other techniques can be used to determine the arrangement of loci on a chromosome and/or map the loci to a chromosome. A chromosomal rearrangement can be, e.g., a deletion, duplication, inversion, or translocation.

A genome can comprise one or more translocations. A translocation can occur when parts between nonhomologous chromosomes are rearranged. A translocation can be a balanced translocation, in which pieces of chromosomes are rearranged but no genetic material is lost or gained in a cell. A translocation can be an unbalanced translocation, in which an exchange of chromosome material is unequal and results in extra or missing genetic material. A translocation can be a reciprocal (non-Robertsonian translocation), which can involve the exchange of material between nonhomologous chromosomes. A translocation can be a Roberstonian translocation. A Robertsonian translocation can involve a rearrangement of two acrocentric chromosomes that fuse near a centromere. Translocations can be associated with cancer, e.g., leukemia (acute myelogenous leukemia and chronic myelogenous leukemia), e.g., solid malignancies such as Ewing's sarcoma.

In some cases, a genome can comprise one or more inversions. An inversion can be a chromosome rearrangement in which a segment of a chromosome is reversed, end to end. An inversion can occur when a single chromosome undergoes breakage and rearrangement within itself. There can be two types of inversions: paracentric and pericentric. A paracentric inversion does not include a centromere; both breaks occur in one arm of the chromosome. A pericentric inversion can include the centromere; a break point exists in each arm.

In some cases, a genome can comprise one or more duplications. A duplication can occur when part of a chromosome is copied, resulting in extra genetic material from the duplicated segment. Duplication can occur through homologous recombination or retrotransposition. In some cases, an entire chromosome is duplicated. Duplication can arise from unequal crossing-over during meiosis between misaligned homologous chromosomes. Duplications can occur in cancer cells. Cancers that can have oncogene amplifications include breast cancer (MYC, ERBB2, CCND1, FGFR1, FGFR2), cervical cancer (MYC, ERBB2), colorectal cancer (HRAS, KRAS, MYB), esophageal cancer (MYC, CCND1, MDM2), gastric cancer (CCNE, KRAS, MET), glioblastoma (ERBB1, CDK4), head and neck cancer (CCND1, ERBB1, MYC), hepatocellular cancer (CCND1), neuroblastoma (MYCN), ovarian cancer (MYC, ERBB2, AKT2), sarcoma (MDM2, CDK4), and small cell lung cancer (MYC).

In some cases, a genome can comprise one or more deletions. A genome deletion can be a mutation in which part of a chromosome or a sequence of DNA is absent from a genome. In some cases, a deletion is a single base, two or more bases, or an entire chromosome. A deletion can result from an error in chromosomal crossover during meiosis, losses from translocation, chromosomal crossover with a chromosomal inversion, unequal crossing over, or breaking of a chromosome without rejoining. In some cases, a deletion can result in a frameshift mutation. In some cases, a deletion is a terminal deletion, which can occur towards an end of a chromosome. In some cases, a deletion is an intercalary deletion or interstitial deletion, which can be a deletion that occurs in the interior of a chromosome. In some cases, a deletion is a microdeletion, which can be a deletion of up to 5000 base pairs.

Confirming Linkage (Haplotype) Information Generated by Digital Experiment

Linkage information can be determined using digital analysis and restriction enzyme digest of samples can be confirmed by one or more other assays. Signal generation during a real-time PCR or ddPCR experiment of a partitioned sample as described herein can be used to confirm linkage information. For example, a sample (e.g., a subsample of a sample used in a linkage experiment) can be partitioned, and PCR can be performed on the partitions (e.g., droplets). The mean fluorescence intensity of partitions can be determined as they undergo exponential amplification for a target and/or reference nucleic acid sequence. Partitions with a polynucleotide with multiple (e.g., 2) linked copies of a target nucleic acid sequence can have higher fluorescence intensity than droplets with only one copy of a target nucleic acid sequence.

Long range PCR can be used to confirm linkage information. For example, PCR can be used to detect the presence of two tandemly arranged copies of a target nucleic acid sequence on the same chromosome (cis-configuration), and it can be used to detect deletion of the target nucleic acid sequence on another chromosome. Primers outside of the amplified region (region suspected of having tandem copies of the target) can be used. DNA polynucleotides can be partitioned into droplets. Partitioning DNA polynucleotides into droplets can be beneficial, as it can permit detection of two types of DNA species: a) the DNA segment with tandemly arranged targets and b) a DNA segment with the deletion of the target. If a similar reaction is performed in bulk (e.g., without partitioning polynucleotides), the smaller PCR product representing the DNA with the deleted target can outcompete the PCR product representing the DNA segment with tandemly arranged target sequences. As a result, only one PCR product can be generated. The size difference of these PCR products can be estimated using, e.g., gel electrophoresis or a Bioanalyzer.

In some cases, DNA with tandemly arranged copies of a target nucleic acid sequence can be too large to be successfully PCR amplified (e.g., >20 KB in size). In these cases, often only the smaller PCR product is amplified, representing the DNA segment with the deleted target nucleic acid sequence. If the target nucleic acid sequence is too long to permit generation of a PCR product, PCR can be performed on a chromosome that contains a deletion for the target nucleic acid sequence. In this case, a product can be generated if the PCR is over a region deleted for the sequence, but a product may not be generated if the target sequence is present because the distance between the primers can be too great.

Long range PCR can be used to resolve linkage or determine copy number estimation. Long range PCR can be used in conjunction with the methods provided herein. Genotypes of parents or other relatives can be used (alone or in combination with the methods provided herein) to infer the copy number state of the target individual.

A chromosomal region can be cloned using recombinant DNA technology and individual copies of the chromosomal region can be sequenced. Next-generation sequencing can be used to identify information related to polymorphisms that are closely spaced (e.g., less than 2000 nucleotides, less than 1000 nucleotides, less than 500 nucleotides, less than 200 nucleotides, or less than 100 nucleotides apart) and are present together in the same sequencing read, and a method provided herein can be used to identify information related to polymorphisms that are further apart (e.g., greater than about 5, 10, 50, 100, 150, 200, 250, 300, 400, 450, 500, 550, 600, 650, 700, 750, 800, 850, 900, 950, 1000, 1250, 1500, 1750, 2000, 2500, 3000, 3500, 4000, 4500, or 5000 nucleotides apart). A method provided herein can be used to identify information related to polymorphisms that are further apart (e.g., greater than about 5, 10, 50, 100, 150, 200, 250, 300, 400, 450, 500, 550, 600, 650, 700, 750, 800, 850, 900, 950, 1000, 1250, 1500, 1750, 2000, 2500, 3000, 3500, 4000, 4500, or 5000 nucleotides apart). In some cases, the method comprises using a method provided herein in conjunction with using genotype information for the parents or other close relatives of a subject to infer phase information using Mendelian rules of inheritance. However, this approach in some cases cannot phase every polymorphism. Some embodiments comprise a method provided herein used in conjunction with statistical approaches to linkage determination.

Haplotypes

A haplotype can refer to two or more alleles that are present together or linked on a single chromosome (e.g., on the same chromosome copy) and/or on the same piece of nucleic acid and/or genetic material. Phasing can be the process of determining whether or not alleles exist together on the same chromosome. Determination of which alleles in a genome are linked can be useful for considering how genes are inherited. The present disclosure provides a system, including method and apparatus, for haplotype analysis by amplification of a partitioned sample.

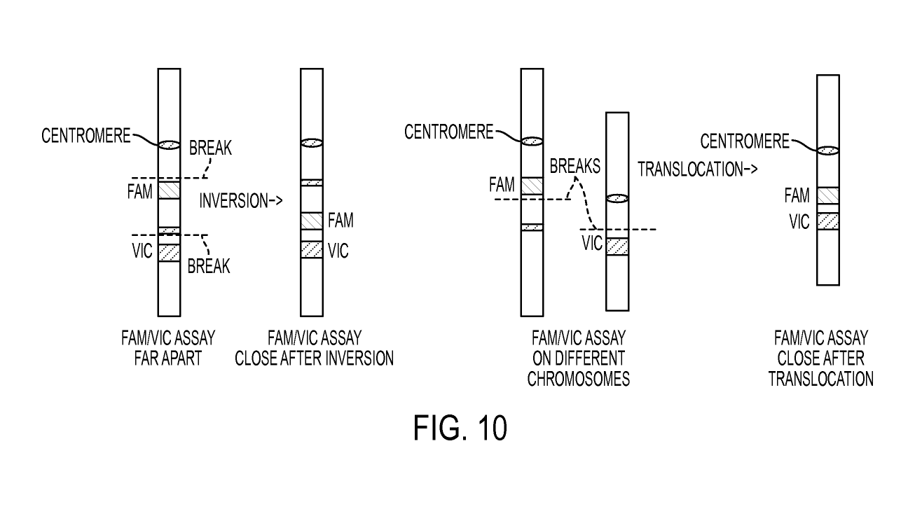

FIG. 11 shows a flowchart listing steps that can be performed in an exemplary method (20) of haplotype analysis. The steps can be performed in any suitable order and combination and can be united with any other steps of the present disclosure. A sample can be obtained (22), generally from a subject with a diploid or higher complement of chromosomes. The sample can be partitioned (24). Partitioning the sample can include partitioning or dividing an aqueous phase that includes nucleic acid of the sample. A pair (or more) of polymorphic loci can be amplified (26). Allele-specific amplification data can be collected for each polymorphic locus (28). Amplification data for the polymorphic loci and from the same volumes can be correlated (30). A haplotype for the polymorphic loci can be selected (32).

Haplotype analysis can be performed with a sample obtained from a subject, such as a person. An aqueous phase containing nucleic acid of the sample can be partitioned into a plurality of discrete volumes, such as droplets. Each volume can contain on average less than one genome equivalent of the nucleic acid, such that each volume contains on average less that about one copy of an allele of a first polymorphic locus and an allele of a linked second polymorphic locus. At least one allele sequence from each of the first polymorphic locus and the second polymorphic locus in the nucleic acid can be amplified. Distinguishable allele-specific amplification data for each of the loci can be collected from individual volumes. Allele-specific amplification data for the first locus can be correlated with allele-specific amplification data for the second locus from the same volumes. A haplotype of the nucleic acid for each of the first and second loci can be selected based on correlation of the allele-specific amplification data. In general, the method can rely on co-amplification, in the same volumes, of allele sequences from distinct loci, if the allele sequences constitute a haplotype of the subject, and, conversely, lack of co-amplification if they do not.

A system for haplotype analysis can comprise a droplet generator configured to form droplets of an aqueous phase including nucleic acid. The system also can comprise a detector configured to collect allele-specific amplification data for each of the loci from individual droplets. The system further can comprise a processor. The processor can be configured to correlate allele-specific amplification data for the first locus with allele-specific amplification data for the second locus from the same volumes and to select a haplotype of the nucleic acid based on correlation of the allele-specific amplification data.

Optionally, the sample may be divided into subsamples. Optionally, the first subsample may be contacted with a restriction enzyme that cleaves a site between the polymorphic loci; and the second subsample may optionally be exposed to a restriction enzyme. Optionally, allele-specific amplification data from the first subsample may be correlated with allele-specific amplification data from the second subsample.

Further aspects of the present disclosure are presented in the following sections: (I) definitions, (II) system overview, (III) exemplary potential haplotypes created by linked SNPs, (IV) exemplary haplotype analysis with amplification in droplets, and (V) selected embodiments.

### I. DEFINITIONS

Technical terms used in this disclosure have the meanings that are commonly recognized by those skilled in the art. However, the following terms may have additional meanings, as described below.

Sequence variation can be any divergence in genome sequence found among members of a population or between/among copies of a chromosome type of a subject and/or a sample. Sequence variation also may be termed polymorphism.

Locus can be a specific region of a genome, generally a relatively short region of less than one kilobase or less than one-hundred nucleotides.

Polymorphic locus can be a locus at which sequence variation exists in the population and/or exists in a subject and/or a sample. A polymorphic locus can be generated by two or more distinct sequences coexisting at the same location of the genome. The distinct sequences can differ from one another by one or more nucleotide substitutions, a deletion/insertion, and/or a duplication of any number of nucleotides, generally a relatively small number of nucleotides, such as less than 50, 10, or 5 nucleotides, among others. A polymorphic locus can be created by a single nucleotide polymorphism (a “SNP”), namely, a single nucleotide position that varies within the population.

Allele can be one of the two or more forms that coexist at a polymorphic locus. An allele also can be termed a variant. An allele can be the major or predominant form or a minor or even very rare form that exits at a polymorphic locus. Accordingly, a pair of alleles from the same polymorphic locus can be present at any suitable ratio in a population, such as about 1:1, 2:1, 5:1, 10:1, 100:1, 1000:1, etc.

Allele sequence can be a string of nucleotides that characterizes, encompasses, and/or overlaps an allele. Amplification of an allele sequence can be utilized to determine whether the corresponding allele is present at a polymorphic locus in a sample partition.

Haplotype can be two or more alleles that are present together or linked on a single chromosome (e.g., on the same chromosome copy) and/or on the same piece of nucleic acid and/or genetic material; haplotype can also refer to two or more target nucleic acids that are present together or linked on a single chromosome. The target nucleic acids can be the same or different.

Linkage can be a connection between or among alleles from distinct polymorphic loci and can also be a connection between or among target nucleic acids that are identical or nearly identical. Polymorphic loci that show linkage (and/or are linked) generally include respective alleles that are present together on the same copy of a chromosome, and can be relatively close to one another on the same copy, such as within about 10, 1, or 0.1 megabases, among others.

In some cases, next generation sequencing can be used to determine a presence or absence of multiple alleles at one or more loci. In some cases, next generation sequencing is used to determine a presence or absence of multiple alleles at one or more loci in a region comprising a copy number variation. A 2-plex, 3-plex, 4-plex, etc. assay can be used to determine whether alleles, e.g., alleles identified by next generation sequencing, at one or more loci are located on the same or different chromosomes. In some cases, digital PCR (e.g., droplet digital PCR) can be used to determine if alleles at different loci are on the same or different chromosomes. In some cases, it is determined whether alleles at at least 2, 3, 4, 5, 6, 7, 8, 9, 10, 11, 12, 13, 14, 15, 16, 17, 18, 19, or 20 loci are on the same or different chromosomes.

### II. SYSTEM OVERVIEW FOR HAPLOTYPE ANALYSIS

FIG. 11 shows a flowchart listing steps that may be performed in an exemplary method 20 of haplotype analysis. The steps may be performed in any suitable order and combination and may be united with any other steps of the present disclosure.

A sample may be obtained, indicated at 22. The sample can be obtained from a subject, generally a subject with a diploid or higher complement of chromosomes. In other words, the subject typically has at least two sets of chromosomes, and at least a pair of each type of chromosome in the subject's cells. For example, somatic cells of humans each contain two copies of chromosome 1, 2, 3, etc., to give 23 chromosome pairs (two sets of chromosomes) and a total of 46 chromosomes.

The sample can be partitioned, indicated at 24. Partitioning the sample can include partitioning or dividing an aqueous phase that includes nucleic acid of the sample. Partitioning divides the aqueous phase into a plurality of discrete and separate volumes, which also can be called partitions. The volumes can be separated from one another by fluid, such as a continuous phase (e.g., an oil). Alternatively, the volumes can be separated from one another by walls, such as the walls of a sample holder. The volumes can be formed serially or in parallel. The volumes can be droplets forming a dispersed phase of an emulsion.

A pair (or more) of polymorphic loci can be amplified, indicated at 26. More particularly, at least one allele sequence from each of the polymorphic loci can be amplified. Each allele sequence can be characteristic of a corresponding allele of the locus. In some cases, only one allele sequence can be amplified from each locus, or a pair of allele sequences can be amplified from at least one of the loci. The particular allele sequences and number of distinct allele sequences that are amplified can be determined by the particular primer sets included in the aqueous phase before the aqueous phase is partitioned.

Allele-specific amplification data can be collected for each polymorphic locus, indicated at 28. The data can relate to distinguishable amplification (or lack thereof) of each of the allele sequences in individual volumes. The data can be detected from distinguishable probes corresponding to and capable of hybridizing specifically to each of the allele sequences amplified. The data can be collected in parallel or serially from the volumes. The data can be collected by optical detection of amplification signals. For example, optical detection can include detecting fluorescence signals representing distinguishable amplification of each allele sequence.

Amplification data for the polymorphic loci and from the same volumes can be correlated, indicated at 30. Correlation generally determines which allele sequences are most likely to be present together in individual volumes, and thus originally linked to one another on the same chromosome copy in genetic material of the subject. Correlation can include determining at least one correlation coefficient corresponding to co-amplification of distinct allele sequences in the same volumes. In some cases, correlation can include determining a pair of correlation coefficients corresponding to co-amplification of each of a pair of allele sequences of the same locus with an allele sequence of another locus. Correlation also can include comparing correlation coefficients with each other and/or with a threshold, or can include determining whether a correlation coefficient is negative or positive. Correlation can be performed with amplification data that has been converted to a binary form by applying a threshold that distinguishes amplification-positive and amplification-negative signals. Correlation also or alternatively can include comparing the numbers of volumes that exhibit co-amplification of different sets of allele sequences and/or comparing the number of volumes that exhibits co-amplification of a set of allele sequences versus the number that exhibits amplification of only one of the allele sequences.

One or both of the steps indicated at 28 and 30 can be substituted by a step of determining at least one measure of co-amplification of allele sequences from both loci in the same volumes. Any suitable measure(s) of co-amplification can be used, such as at least one correlation coefficient obtained by correlation of allele-specific amplification data for the polymorphic loci from the same volumes. In other examples, the measure of co-amplification can be at least one value representing at least one number or frequency of co-amplification of an allele sequence from each locus. Further aspects of correlating amplification data and determining measures of co-amplification are described elsewhere in the present disclosure, such as in Section IV.

The sample containing polynucleotides can be divided into two or more subsamples. The first subsample can be exposed to a restriction enzyme which cleaves at a site between the two polymorphic loci. The first subsample can then be partitioned into multiple partitions. Allele-specific amplification data can then be collected for each polymorphic locus, as described herein. The second subsample, having not been exposed to a restriction enzyme which cleaves at a site between the two polymorphic loci, can be partitioned into multiple partitions. Allele-specific amplification can then be collected for each polymorphic locus. Amplification data from the first and second subsamples can be correlated to determine the haplotype for the polymorphic loci.

A haplotype for the polymorphic loci can be selected, indicated at 32. Selection can be based on correlation of amplification data and/or based on the at least one measure of co-amplification. The haplotype can be selected from among a set of potential haplotypes for the polymorphic loci being investigated. The selected haplotype generally includes designation of at least a pair of particular alleles that are likely to be linked to one another on the same chromosome copy of the subject.

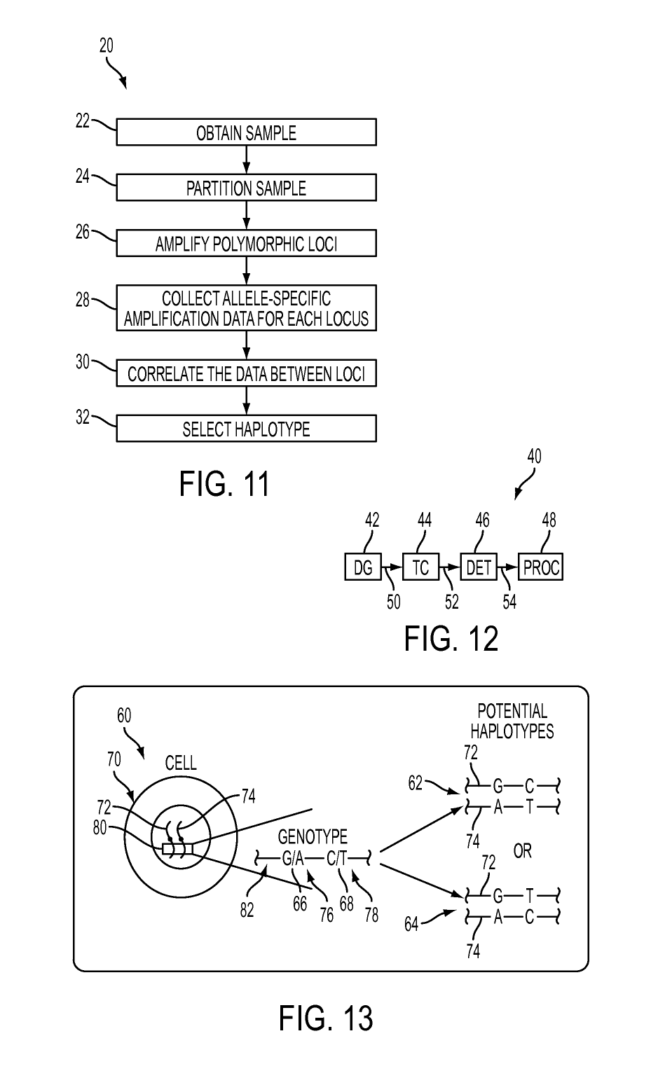

FIG. 12 shows a schematic view of selected aspects of an exemplary system 40 for performing method 20 of FIG. 11. The system may include a droplet generator (DG) 42, a thermocycler (TC) 44, a detector (DET) 46, and a processor (PROC) 48. Arrows 50-54 extend between system components to indicate movement of droplets (50 and 52) and data (54), respectively.

Droplet generator 42 can form droplets of an aqueous phase including nucleic acid. The droplets can be formed serially or in parallel.

Thermocycler 44 can expose the droplets to multiple cycles of heating and cooling to drive amplification, such as PCR amplification, of allele sequences. The thermocycler can be a batch thermocycler, which can amplify all of the droplets in parallel, or can be a flow-based thermocycler, which amplifies droplets serially, among others.

Detector 46 collects amplification data, such as allele-specific amplification data from the droplets. The detector can, for example, be a fluorescence detector, and can detect droplets serially or in parallel.

Processor 48, which also can be termed a controller, can be in communication with detector 46 and can be programmed to process amplification data from the detector. The processor, which can be a digital processor, can be programmed to process raw data from the detector, such as to subtract background and/or normalize droplet data based on droplet size. The processor also or alternatively can be programmed to apply a threshold to convert the data to binary form, to perform a correlation of amplification data, to calculate and/or compare one or more measures of co-amplification, to select a haplotype based on the correlation and/or measures, or any combination thereof.

Further aspects of droplet generators, thermocyclers, detectors, and controllers are described in U.S. Patent Application Publication No. 2010/0173394 A1, published Jul. 8, 2010, which is incorporated herein by reference.

### III. EXEMPLARY POTENTIAL HAPLOTYPES CREATED BY LINKED SNPS

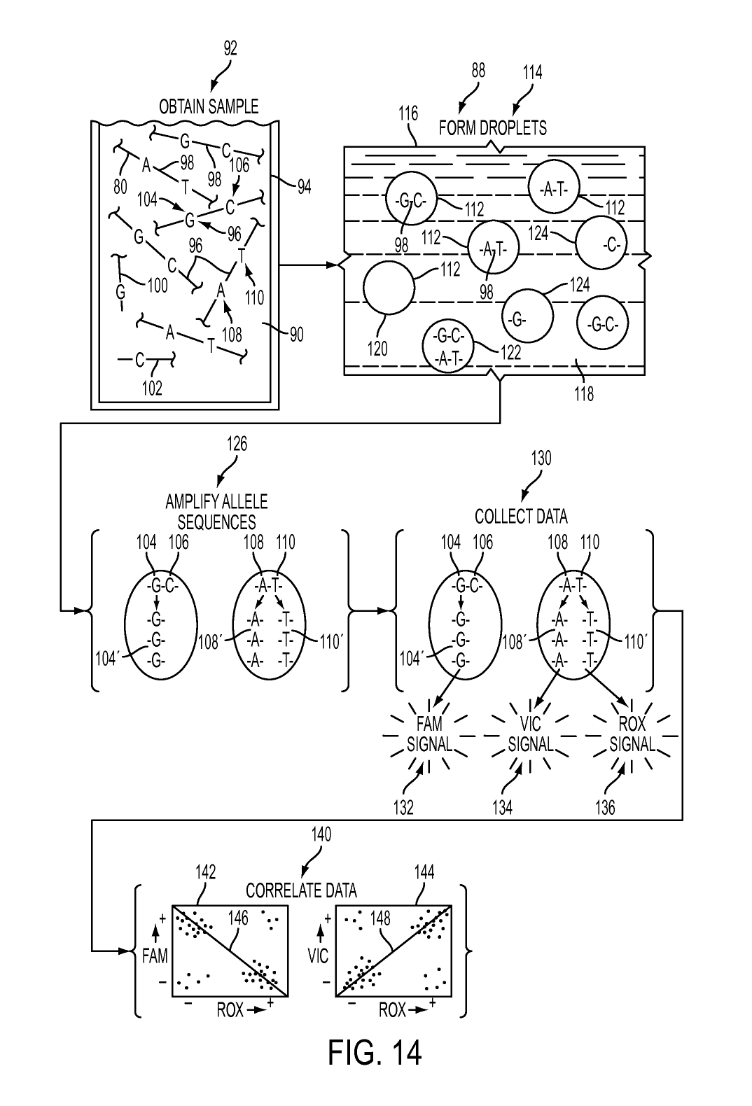

FIG. 13 schematically illustrates a haplotyping situation created by linked SNPs in which the genetic material of a diploid subject 60 has two different nucleotides at each of two different loci. The goal of haplotyping is to determine which nucleotide at the first locus is combined with which nucleotide at the second locus on each chromosome copy.

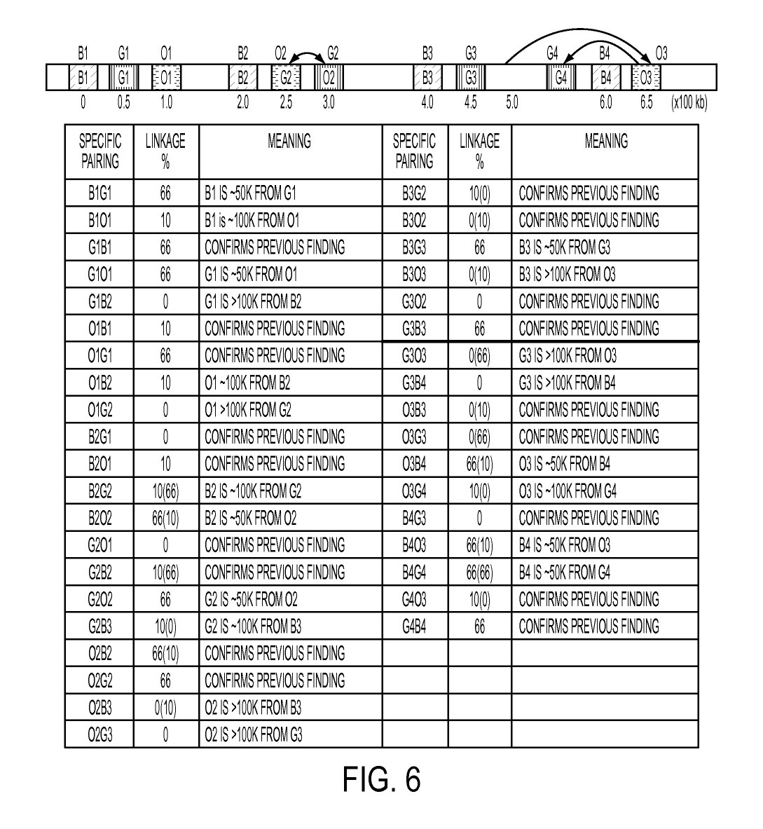

Subject 60 can have either of two alternative haplotype configurations 62, 64 created by a pair of single nucleotide polymorphisms 66, 68. Each configuration represents two haplotypes: configuration 62 has haplotypes (G, C) and (A, T), and configuration 64 has haplotypes (G, T) and (A, C). A cell 70 of the subject includes a pair of chromosome copies 72, 74 of the same type. (Other types of chromosomes that can be present in the cell are not shown.) Chromosome copies 72, 74 can be mostly identical in sequence to each other, but the copies also typically have many loci of sequence variation, such as polymorphic loci 76, 78, where the two chromosome copies differ in sequence. Loci 76, 78 are contained in a genome region or target region 80, which is outlined by a dashed box in the nucleus of cell 70 and which is shown enlarged adjacent the cell as a composite sequence that represents a genotype 82 for loci 76, 78. (Only one strand of each chromosome copy and target region is shown in FIG. 6 (and FIG. 7) to simplify the presentation.)

Genotype 82 can be determined by any suitable genotyping technology, either before haplotype analysis or as a part of a haplotype analysis. Genotype 82 shows that the single polymorphic nucleotide of locus 76 is a “G” and an “A” on chromosome copies 72 and 74 (or vice versa), and for locus 78 is a “C” and a “T.” However, the genotype does not indicate how the individual nucleotides of the two loci are combined on chromosome copies 72, 74. Accordingly, the genotype can be produced by alternative, potential haplotype configurations 62, 64. Haplotype analysis, as disclosed herein, permits determination of which of the potential haplotypes are present in the subject s genetic material.

### IV. EXEMPLARY HAPLOTYPE ANALYSIS WITH AMPLIFICATION IN DROPLETS

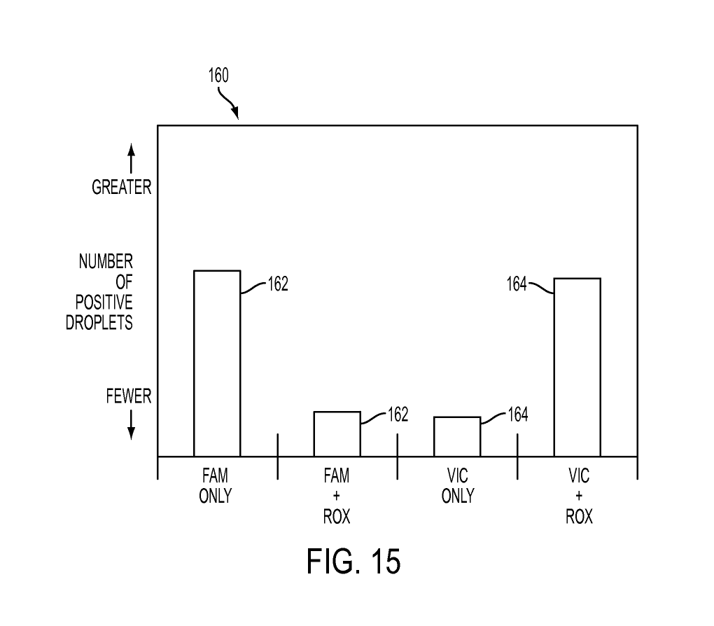

FIG. 14 schematically illustrates performance of an exemplary version 88 of the method of FIG. 11. Here, genetic material from the subject of FIG. 13 is analyzed to distinguish the alternative, potential haplotype configurations described in the preceding section.

A sample 90 is obtained, indicated at 92. The sample is disposed in an aqueous phase 94 including nucleic acid 96 of the subject. In this view, for simplification, only fragments 98 containing genome region 80 are depicted. Fragments 98 are long enough that only a minority (e.g., incomplete fragments 100, 102) fail to include an allele sequence 104-110 from both loci 76, 78 (also see FIG. 13). The aqueous phase may be configured for PCR amplification of allele sequences 104-110.

Droplets 112 are formed, indicated at 114. The droplets may be part of an emulsion 116 that includes a continuous phase 118 separating the droplets from one another. The droplets may be monodisperse, that is, of substantially the same size. Exemplary degrees of monodispersity that may be suitable are described in U.S. Patent Application Publication No. 2010/0173394 A1, published Jul. 8, 2010, which is incorporated herein by reference.

Fragments 98 may distribute randomly into the droplets as they are formed. At a proper dilution of fragments 98 in the aqueous phase that is partitioned, and with a proper selection of droplet size, an average of less than one copy or molecule of target region 80 is contained in each droplet. Accordingly, some droplets, such as the empty droplet indicated at 120, contain no copies of the target, many contain only one copy of the target region, some contain two or more copies of the target (e.g., the droplet indicated at 122), and some contain only one of the allele sequences of the target region (e.g., the droplets indicated at 124).

Allele sequences can be amplified, indicated at 126. Here, two allele sequences, 104 and 108, are amplified from locus 76 and only allele sequence 110 is amplified from locus 78 (also see FIG. 13). Amplified copies of each allele sequence are indicated at 104′, 108′, and 110′. In other embodiments, only one allele sequence may be amplified from each locus, or at least two allele sequences may be amplified from each locus, among others. (For example, allele sequence 106 may be amplified with the same primers that amplify allele sequence 110, but amplification of allele sequence 106 is not shown here to simplify the presentation.)

Allele-specific amplification data can be collected from the droplets, indicated at 130. In this example, fluorescence data is collected, with a different, distinguishable fluorescent dye, each included in a different allele-specific probe, providing amplification signals for each allele sequence 104′, 108′, 110′. In particular, the dyes FAM, VIC, and ROX emit FAM−, VIC−, and ROX signals 132-136 that relate to amplification of allele sequences 104, 108, and 110, respectively. In other embodiments, allele-specific amplification of all four allele sequences 104-110 or of only two allele sequences (one from each locus) may be detected.

The amplification data is correlated, indicated at 140, and/or at least one measure of co-amplification of allele sequences in the same droplets is determined Graphs 142, 144 schematically illustrate an approach to correlation and/or determination of measures of co-amplification. Graph 142 plots FAM and ROX signal intensities for individual droplets (represented by dots in the plot), while graph 144 plots VIC and ROX signal intensities for individual droplets. Signal values that represent amplification-negative (“−”) and amplification-positive (“−”) droplets for a given signal type (and thus a given allele sequence) are indicated adjacent each axis of the graphs.

Lines 146, 148 represent a best-fit of the amplification data of each graph to a linear relationship. However, the two fits have associated correlation coefficients of opposite polarity. The amplification data in graph 142 provides a negative correlation coefficient, because there is a negative correlation for co-amplification of allele sequence 104 (as reported by FAM signals) and allele sequence 110 (as reported by ROX signals) in the same droplets. In contrast, the amplification data in graph 144 provides a positive correlation coefficient, because there is a positive correlation for co-amplification of allele sequence 108 (as reported by VIC signals) and allele sequence 110 (as reported by ROX signals) in the same droplets. The correlation coefficients may be compared to one another to select a haplotype. For example, the haplotype may be selected based on which correlation coefficient is larger (e.g., closer to 1.0) and/or which is positive (if only one is positive). Here, a first haplotype including allele sequences 104 and 106 and a second haplotype including allele sequences 108 and 110 may be selected based on the positive correlation of VIC and ROX signals. In some embodiments, a haplotype may be selected based on only one correlation, such as based on whether a correlation coefficient is negative or positive or based on comparison of the correlation coefficient to a predefined value.

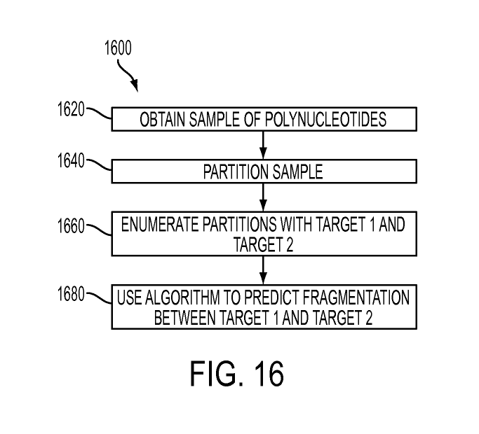

FIG. 15 shows a bar graph 160 illustrating an alternative approach to correlating the amplification data of FIG. 14. The amplification data of FIG. 14 has been converted to binary form by comparing each type of droplet signal (FAM, VIC, and ROX) with a threshold that distinguishes amplification-positive droplets (assigned a “1”) from amplification-negative droplets (assigned a “0”) for each allele sequence. Graph 160 tabulates the binary form of the data to present the number of amplification-positive droplets for various allele sequences alone or in combination. The leftward two bars, indicated at 162, allow a comparison of the number of droplets that contain only allele sequence 104 (FAM) with the number that contain both allele sequences 104 (FAM) and 110 (ROX). The leftward data shows that amplification of allele sequence 104 does not correlate well with amplification of allele sequence 110. In other words, allele sequences 104 and 110 tend not to be co-amplified in the same droplets. The rightward two bars, indicated at 164, allow a comparison of the number of droplets that contain only allele sequence 108 (VIC) with the number that contain both allele sequences 108 (VIC) and 110 (ROX). The rightward data shows that amplification of allele sequence 108 correlates well with amplification of allele sequence 110. In other words, allele sequences 108 and 110 tend to be co-amplified in the same droplets. The leftward pair of bars and the rightward pair of bars considered separately or collectively indicate a haplotype in which allele sequence 108 is associated with allele sequence 110.

A sample comprising genetically linked loci can be subjected to fragmentation before being analyzed by the methods, compositions, or kits described herein. A sample comprising genetically linked loci can be fragmented by, e.g., mechanical shearing, passing the sample through a syringe, sonication, heat treatment (e.g., 30 mins at 90° C.), and/or nuclease treatment (e.g., with DNase, RNase, endonuclease, exonuclease, restriction enzyme). A sample comprising genetically linked loci can be subjected to no or limited processing before being analyzed.

In another embodiment, using droplet digital PCR (ddPCR), a duplex reaction can be performed targeting two genomic loci, e.g., two genes on a common chromosome. The droplets can be categorized into four populations according to their fluorescence. For example, if a FAM-labeled probe is used to detect to one loci, and a VIC-labeled probe is used to detect another loci, the four populations can be FAM+/VIC+, FAM+/VIC−, FAM−/VIC+, and FAM−/VIC−. By comparing the number of droplets with each of these populations, it can be possible to determine the frequency at which loci co-segregate to the same droplet. Using Poisson statistics, the percentage of species that are actually linked to one another can be estimated versus instances where two separated loci are in the same droplet by chance.

The number of genetically linked loci that can be examined to determine if they are still linked in a sample or are separated in the sample using the methods, compositions, and kits described herein can be about, at least, or more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. The number of genetically linked loci that can be examined to determine if they are still linked in a sample or are separated in the sample using the methods, compositions, and kits described herein can be about 2 to about 10, about 2 to about 8, about 2 to about 6, about 2 to about 4, about 3 to about 10, about 3 to about 8, about 3 to about 6, about 4 to about 10, about 4 to about 6, about 10 to about 100, about 10 to about 50, about 10 to about 25, about 10 to about 20, about 5 to about 100, about 5 to about 50, about 5 to about 25, about 5 to about 20, about 5 to about 15, or about 5 to about 10.

The number of base pairs between each of the genetically linked loci can be about, at least, more than, or less than 10 bp, 25 bp, 50 bp, 75 bp, 100 bp, 250 bp, 500 bp, 750 bp, 1000 bp, 2000 bp, 3000 bp, 4000 bp, 5000 bp, 6000 bp, 7000 bp, 8000 bp, 9000 bp, 10,000 bp, 15,000 bp, 20,000 bp, 33,000 bp, 50,000 bp, 75,000 bp, 100,000 bp, 250,000 bp, 500,000 bp, 750,000 bp, 1,000,000 bp, 1,250,000 bp, 1,500,000 bp, 2,000,000 bp, 5,000,000 bp, or 10,000,000 bp. The number of base pairs between each of the genetically linked loci can be about 10 to about 10,000,000 bp, about 100 to about 10,000,000 bp, about 1,000 to about 10,000,000 bp, about 1,000 to about 1,000,000 bp, about 1,000 to about 500,000 bp, about 1,000 to about 100,000 bp, about 3000 to about 100,000 bp, about 1000 to about 33,000 bp, about 1,000 to about 10,000 bp, or about 3,000 to about 33,000 bp. The number of base pairs between each genetically linked alleles can be 0 bp.

A method of haplotying can comprise examining if two alleles at two different loci co-localize to the same spatially-isolated partition. Additional alleles at the two loci can be analyzed. For example, if two alleles at two different loci do not co-localize in a digital experiment, one or more other alleles at the two loci can be analyzed to provide a positive control for colocalization. For example, assume a maternally-inherited chromosome has allele A is at locus 1 and allele Y is at locus 2, 100 bp away from locus 1. On the corresponding paternally-inherited chromosome, assume allele B is at locus 1 and allele Z is at locus 2. If a nucleic acid sample comprising these nucleic acids is separated into spatially isolated partitions, and amplification for allele A and allele Z is performed, the amplification signal for allele A and allele Z should rarely or never colocalize to a single partition because allele A and allele Z are not linked. A digital analysis can be performed to confirm that allele A and allele Y are linked on the maternally-inherited chromosome or that allele B and allele Z are linked on the paternally-inherited chromosome.

Haplotyping with Two Colors

While embodiments shown herein demonstrate the use of a three-color system to measure phasing, phasing may also be measured using a two color system. For example, if two heterozygous SNPs (Aa and Bb) need to be phased, one can use an assay with a FAM-labeled probe targeting A and an assay with a VIC-labeled probe targeting B. Excess of partitions containing both A and B would be indicative of linkage between A and B, suggesting that the two haplotypes are A-B and a-b. Absence of such excess may be suggestive of the alternative combination of haplotypes: A-b and a-B. One can determine that the DNA is of high enough molecular weight to make this later inference. In order to confirm the alternative combination of haplotypes, another duplex assay can be run in a separate well, where a different combination of alleles is targeted. For example, a FAM assay can be run targeting A and a VIC assay can be run targeting b. Excess of partitions containing both A and b would be indicative of linkage between A and b, suggesting that the two haplotypes are A-b and a-B.

Reference Sequences

In methods involving the analysis of copy number (or other applications described herein), it can be useful to count the number of times a particular sequence (e.g., target) is found, e.g., in a given genome. This analysis can be done by assessing (or comparing) the concentrations of a target nucleic acid sequence and of a reference nucleic acid sequence known to be present at some fixed number of copies in every genome. For the reference, a housekeeping gene (e.g., a gene that is required for the maintenance of basic cellular function) can be used that is present at two copies per diploid genome. Dividing the concentration or amount of the target by the concentration or amount of the reference can yield an estimate of the number of target copies per genome. One or more references can also be used to determine target linkage.

A housekeeping gene that can be used as reference in the methods described herein can include a gene that encodes a transcription factor, a transcription repressor, an RNA splicing gene, a translation factor, tRNA synthetase, RNA binding protein, ribosomal protein, RNA polymerase, protein processing protein, heat shock protein, histone, cell cycle regulator, apoptosis regulator, oncogene, DNA repair/replication gene, carbohydrate metabolism regulator, citric acid cycle regulator, lipid metabolism regulator, amino acid metabolism regulator, nucleotide synthesis regulator, NADH dehydrogenase, cytochrome C oxidase, ATPase, mitochondrial protein, lysosomal protein, proteosomal protein, ribonuclease, oxidase/reductase, cytoskeletal protein, cell adhesion protein, channel or transporter, receptor, kinase, growth factor, tissue necrosis factor, etc. Specific examples of housekeeping genes that can be used in the methods described include, e.g., HSP90, Beta-actin, tRNA, rRNA, ATF4, RPP30, and RPL3.

For determining the linkage of a target, one of the loci genetically linked to another locus can be a common reference, e.g., RPP30. Any genetically linked loci can be used in the methods described herein.

A single copy reference nucleic acid (e.g., gene) can be used to determine copy number variation. Multi-copy reference nucleic acids (e.g., genes) can be used to determine copy number to expand the dynamic range. For example, the multi-copy reference gene can comprise about, or more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 500, 1000, 2000, 3000, 4000, 5000, 6000, 7000, 8000, 9000, 10,000, 20,000, 30,000, 40,000, 50,000, 60,000, 70,000, 80,000, 90,000, or 100,000 copies in a genome. Multiple different nucleic acids (e.g., multiple different genes) can be used as a reference.

Determining Probability of Nucleic Acid Fragmentation

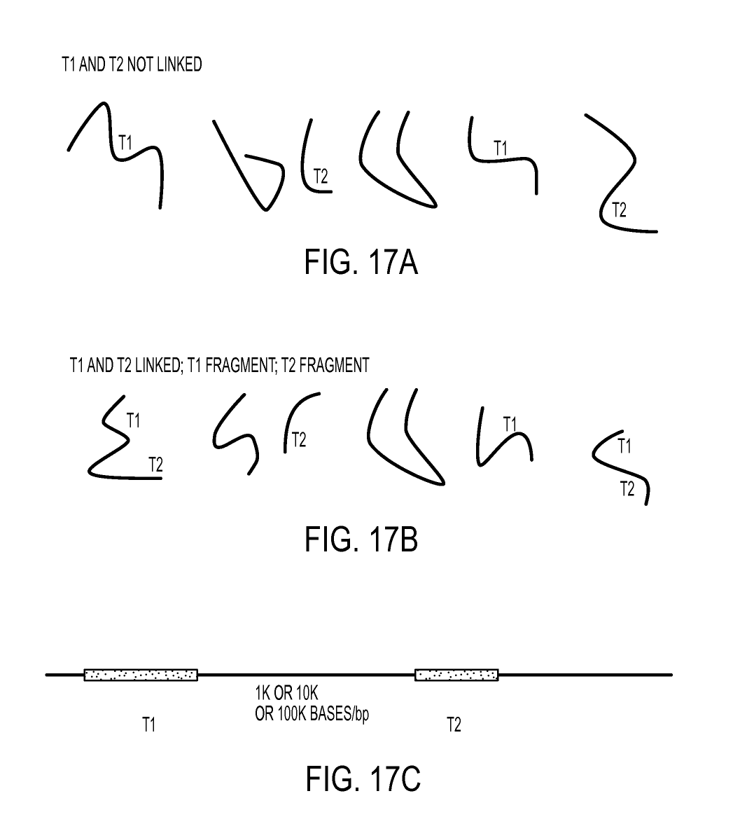

Digital analysis can be performed to determine the extent of fragmentation between two markers in a nucleic acid sample. FIG. 16 illustrates a workflow (1600). The steps in FIG. 16 can be performed in any suitable order and combination and can be united with any other steps of the present disclosure. A sample of polynucleotides can be obtained (1620). The sample can be partitioned into a plurality of partitions (1640) such that each partition contains on average only about 0, 1, 2, or several target polynucleotides. Each partition can have, on average, less than 5, 4, 3, 2, or 1 copies of a target nucleic acid per partition (e.g., droplet). In some cases, at least 0, 1, 2, 3, 4, 5, 6, 7, 8, 9, 10, 20, 30, 40, 50, 60, 70, 80, 90, 100, 125, 150, 175, or 200 partitions (e.g., droplets) have zero copies of a target nucleic acid.

The partitions can be assayed to enumerate partitions with a first target and a second target sequence (1660) and an algorithm can be used to predict fragmentation between the first and second target sequence (1680).

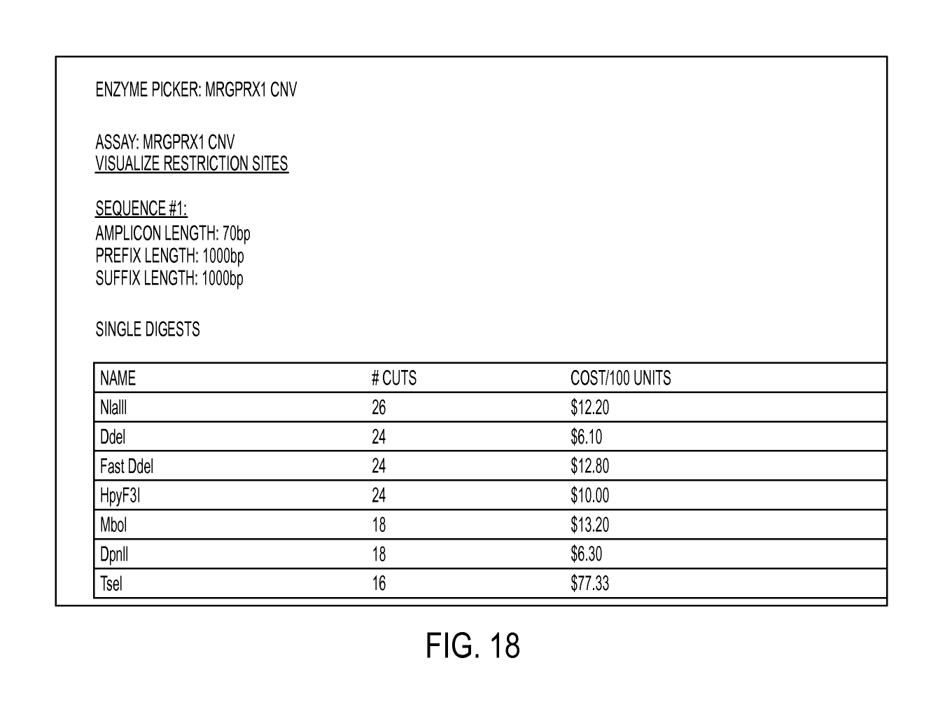

If two different loci (T1 and T2) are on different polynucleotides, a sample with the polynucleotides (1620) will contain polynucleotides with T1 only and T2 only (see FIG. 17A). However, if T1 and T2 are on the same polynucleotide, a sample containing polynucleotides with T1 and T2 can have three species: fragmented polynucleotides with T1, fragmented polynucleotides with T2, and fragmented polynucleotides with T1 and T2 (FIG. 17B). The longer the distance between T1 and T2, the higher the probability of fragmentation between T1 and T2. The sample can be partitioned (FIG. 16: 1640). A digital analysis can be performed, such as digital PCR or droplet digital PCR, and partitions with signal for T1, T2, and T1 and T2 can be enumerated (1660). An algorithm can be developed and used to determine the probability of fragmentation between T1 and T2 (1680). The algorithm can make use of the number of bases or base pairs between T1 and T2 if known. This method can be used to determine the extent of fragmentation of a DNA sample. If there are a number of partitions containing signal for T1 and T2 is greater than the number of partitions one would expect T1 and T2 to be in the same partition, this observation can indicate that T1 and T2 are linked.

It can be advantageous to use the above methods on a nucleic acid (e.g., DNA) sample to ensure that DNA is of high enough molecular weight that linkage information is preserved in the sample.

In any of the methods described herein making use of DNA, an assay can be performed to estimate the fragmentation of the DNA in the sample, and the methods can incorporate the information on fragmentation of the DNA. In another embodiment, results of an assay can be normalized based on the extent of fragmentation of DNA in a sample.

Nucleic acid fragmentation can also be measured by, e.g., gels, a Bioanalyzer, or size exclusion chromatography.

Separation

Physical separation of target sequences can occur in a sequence-specific or non-sequences specific manner. Nonsequence specific means for separating target sequences include use of a syringe, sonication, heat treatment (e.g., 30 mins at 90° C.), and some types of nuclease treatment (e.g., with DNase, RNase, endonuclease, exonuclease).

Restriction Enzymes

A sequence specific method of separation of nucleic acid sequences can involve use of one or more restriction enzymes. One or more restriction enzymes can be used in any of the methods described herein. For example, restriction enzymes can be used to separate target copies in order to estimate copy number states accurately, to assess phasing, to generate haplotypes, or determine linkage, among other methods. One or more enzymes can be chosen so that the nucleic acid (e.g., DNA or RNA) between the target nucleic acids sequences is restricted, but the regions to be amplified or analyzed are not. In some embodiments, restriction enzymes can be chosen so that the restriction enzyme does cleave within the target sequence, e.g., within the 5′ or 3′ end of a target sequence. For example, if target sequences are tandemly arranged without spacer sequence, physical separation of the targets can involve cleavage of sequence within the target sequence. The digested sample can be used in the digital analysis (e.g., ddPCR) reaction for copy number estimation, linkage determination, haplotyping, examining RNA or DNA degradation, or determining methylation burden, e.g., of a CpG island.

Restriction enzymes can be selected and optimal conditions can be identified and validated across numerous sample and assay types for broad applications, e.g., digital PCR (e.g., ddPCR) for CNV determinations and any of the other methods described herein.

Computer software can be used to select one or more restriction enzymes for the methods, compositions, and/or kits described herein. For example, the software can be Qtools software.

One or more restriction enzyme used in the methods, compositions, and/or kits described herein can be any restriction enzyme, including a restriction enzyme available from New England BioLabs®, Inc. (see www.neb.com). A restriction enzyme can be, e.g., a restriction endonuclease, homing endonuclease, nicking endonuclease, or high fidelity (HF) restriction enzyme. A restriction enzyme can be a Type I, Type II, Type III, or Type IV enzyme or a homing endonuclease. In some cases, the restriction digest occurs under conditions of high star activity. In some cases, the restriction digest occurs under conditions of low star activity.

A Type I enzyme can cleave at sites remote from the recognition site; can require both ATP and S-adenosyl-L-methionine to function; and can be a multifunctional protein with both restriction and methylase activities. The recognition sequence for a Type I restriction endonuclease can be bipartite or interrupted. The subunit configuration of a restriction endonuclease can be a penatmeric complex. Coactivators and activators of Type I restriction endonucleases include, e.g., magnesium, AdoMet (S-Adenosyl methionine; SAM, SAMe, SAM-e), and ATP. Type I restriction endonucleases can cleave at a cleavage site distant and variable form the recognition site. Examples of Type I restriction endonucleases can include, e.g., EcoKI, EcoAI, EcoBI, CfrAI, StyLTII, StyLTIII, and StySPI.

A Type II enzyme can cleave within or at short specific distances from a recognition site; can require magnesium; and can function independent of methylase. The recognition sequence for a Type II restriction endonuclease can be palindromic or an interrupted palindrome. The subunit structure of a Type II restriction endonuclease can be a homodimer Cleavage of a cleavage site with a Type II restriction endonuclease can result in fragments with a 3′ overhang, 5′ overhang, or a blunt end. Examples of Type II restriction endonucleases include, e.g., EcoRI, BamHI, KpnI, NotI, PstI, SmaI, and XhoI.

There are several subtypes of Type II restriction enzymes, including Type II, Type IIs, and Type IIe.

A Type IIb restriction endonuclease can have a recognition sequence with is bipartite or interrupted. The subunit structure of a Type IIb restriction endonuclease can be a heterotrimer. Cofactors and activators of Type IIb restriction endonucleases can include magnesium and AdoMet (for methylation). A Type IIb restriction endonuclease can cleave at a cleavage site on both strands on both sides of a recognition site a defined, symmetric, short distance away and leave a 3′ overhang. Examples of Type IIb restriction endonucleases include, e.g., BcgI, Bsp24I, CjeI, and CjePI.

A Type IIe restriction endonuclease can have a recognition site that is palindromic, palindromic with ambiguities, or non-palindromic. The subunit structure of a Type IIe restriction endonuclease can be a homodimer or monomer. Cofactors and activators of Type IIe restriction endonuclease can include magnesium, and a second recognition site that can act in cis or trans to the endonuclease can act as an allosteric affector. A Type IIe restriction enzyme can cleave a cleavage site in a defined manner with the recognition sequence or a short distance away. Activator DNA can be used to complete cleavage. Examples of Type IIe restriction enzymes include, e.g., NaeI, BspMI, HpaII, Sa II, EcoRII, Eco57I, AtuBI, Cfr9I, SauBMKI, and Ksp632I.

A Type IIs restriction enzyme can have a recognition sequence that is non-palindromic. The recognition sequence can be contiguous and without ambiguities. The subunit structure of a Type IIs restriction endonuclease can be monomeric. A cofactor that can be used with a Type IIs restriction enzyme can be magnesium. A Type IIs restriction enzyme can cleave at a cleavage site in a defined manner with at least one cleavage site outside the recognition sequence. Examples of Type IIs restriction enzymes include, e.g., FokI, Alw26I, BbvI, BsrI, EarI, HphI, MboII, SfaNI, and Tth111I.

A Type III enzyme can cleave at a short distance from a recognition site and can require ATP. S-adenosyl-L-methionine can stimulate a reaction with a Type III enzyme but is not required. A Type III enzyme can exist as part of a complex with a modification methylase. The recognition sequence of a Type III restriction endonuclease can be non-palindromic. Cofactors and activators that can be used with Type III restriction endonucleases include, e.g., magnesium, ATP (not hydrolyzed), and a second unmodified site in the opposite orientation, a variable distance away. Examples of Type III restriction endonucleases include, e.g., EcoP15I, EcoPI, HinfIII, and StyLTI.

A Type IV enzyme can target methylated DNA. Examples of Type IV restriction enzymes include, e.g., McrBC and Mrr systems of E. coli.

The restriction enzyme can be a homing endonuclease. A homing endonuclease can be a double stranded DNase. A homing endonuclease can have large, asymmetric recognition sites (e.g., 12-40 base pairs). Coding sequences for homing endonucleases can be embedded in introns or inteins. An intein can be a “protein intron” that can excise itself and rejoin the remaining portions (the exteins) with a peptide bond. A homing endonuclease can tolerate some sequence degeneracy within its recognition sequence. The specificity of a homing endonuclease can be 10-12 base pairs. Examples of homing endonucleases include I-CeuI, I-SceI, I-PpoI, PI-SceI, PI-PspI, and PI-SceI.

A restriction enzyme used in the methods, compositions, and/or kits herein can be a dimer, trimer, tetramer, pentamer, hexamer, etc.

The one or more restriction enzymes used in the methods, compositions and/or kits described herein can be a component of a hybrid or chimeric protein. For example, a domain of a restriction enzyme comprising an enzymatic activity (e.g., endonuclease activity) can be fused to another protein, e.g., a DNA binding protein. The DNA binding protein can target the hybrid to a specific sequence on a DNA. The nucleic acid cleavage activity of the domain with enzymatic activity can be sequence specific or sequence non-specific. For example, the non-specific cleavage domain from the type IIs restriction endonuclease Fold can be used as the enzymatic (cleavage) domain of the hybrid nuclease. The sequence the domain with the enzymatic activity can cleave can be limited by the physical tethering of the hybrid to DNA by the DNA binding domain. The DNA binding domain can be from a eukaryotic or prokaryotic transcription factor. The DNA binding domain can recognize about, or at least, 2, 3, 4, 5, 6, 7, 8, 9, 10, 11, 12, 13, 14, 15, 16, 17, 18, 19, 20, 21, 22, 23, 24, or 25 base pairs of continuous nucleic acid sequence. In some cases, the restriction enzyme is a 4-base cutter, 6-base cutter, or 8-base cutter. The DNA binding domain can recognize about 9 to about 18 base pairs of sequence. The DNA binding domain can be, e.g., a zinc finger DNA binding domain. The hybrid can be a zinc finger nuclease (e.g., zinc finger nuclease). The hybrid protein can function as a multimer (e.g., dimer, trimer, tetramer, pentamer, hexamer, etc.).

Examples of specific restriction enzymes that can be used in the methods, compositions, and/or kits described herein include AaaI, AagI, AarI, AasI, AatI, AatII, AauI, AbaI, AbeI, AbrI, AccI, AcpII, AccIII, Acc16I, Acc36I, Acc65I, Acc113I, AccB1I, AccB2I, AccB7I, AccBSI, AccEBI, AceI, AceII, AceIII, AciI, AclI, AclNI, AclWI, AcpI, AcpII, AcrII, AcsI, AcuI, AcvI, AcyI, AdeI, AeuI, AfaI, Afa22MI, Afa16RI, AfeI, AflI, AflII, AflIII, AgeI, AgeI-HF, AglI, AhaI, AhalI, AhaIII, AhaB8I, AhdI, AhlI, AhyI, AitI, AjnI, AjoI, AleI, AlfI, AliI, AliAJI, AloI, AluI, AlwI, Alw21I, Alw261, Alw441, AlwNI, AlwXI, Ama87I, AcoI, AocII, AorI, Aor13HI, Aor51HI, AosI, AosII, ApaI, ApaBI, ApaCI, ApaLI, ApaORI, ApeKI, ApiI, ApoI, ApyI, AquI, AscI, AseI, AselII, AsiSI, AvaI, AvaII, AvrII, BaeGI, BaeI, BamHI, BamHI-HF, BanI, BanII, BbsI, BbvCI, BbvI, BccI, BceAi, BcgI, BciVI, BcII, BcoDI, BfaI, BfuAI, BfuCI, BglI, BglII, BlpI, BmgBI, BmrI, BmtI, BpmI, Bpu10I, BpuEI, BsaAI, BsaBI, BsaHI, BsaI, BsaI-HF, BsaJI, BsaWI, BsaXI, BseRI, BseYI, BsgI, BsiEI, BsiHKAI, BsiWI, BsII, BsmAI, BSmBI, BsmFI, BsmI, BsoBI, Bsp1286I, BspCNI, BspDI, BspEI, BspHI, BspMI, BspQI, BsrBI, BsrDI, BsrFI, BsrGI, BsrI, BssHII, BssKI, BssSI, BstAPI, BstBI, BsteII, BstNI, BstUI, BstXI, BstYI, BstZ17I, Bsu36I, BtgI, BtgZI, BtsCI, BtsI, BtsIMutI, Cac8I, ClaI, CspCI, CviAII, CviKI-1, CviQI, DdeI, DpnI, DpnII, DraI, DraIII, DraIII-HF™, DrdI, EaeI, EagI, EagI-HF™, EarI, EciI, Eco53kI, EcoNI, Eco0109I, EcoP15I, EcoRI, EcoRI-HF™, EcoRV, EcoRV-HF™, FatI, FauI, Fnu4HI, FokI, FseI, FspEI, FspI, HaeII, HaeIII, HgaI, HhaI, HincII, HindIII, HindIII-HF™, Hinfl, HinP1I, HpaI, HpaII, HphI, Hpy166II, Hpy188I, Hpy188III, Hpy99I, HpyAV, HpyCH4III, HpyCH4IV, HpyCH4V, I-CeuI, I-SceI, KasI, KpnI, KpnI-HF™, LpnPI, MboI, MboII, MfeI, MfeI-HF™, MluCI, MluI, MmeI, MnII, MscI, MseI, MslI, MspAlI, MspI, MspJI, MwoI, NaeI, NarI, Nb.BbvCI, Nb.BsmI, Nb.BsrDI, Nb.BtsI, NciI, NcoI, NcoI-HF™, NdeI, NgoMIV, NheI, NheI-HF™, NlaIII, NlaIV, NmeAIII, NotI, NotI-HF™, NruI, NsiI, NspI, Nt.AlwI, Nt.BbvCI, Nt.BsmAI, Nt.BspQI, Nt.BstNBI, Nt.CviPII, PacI, PaeR7I, PciI, PflFI, PflMI, PhoI, PI-PspI, PI-SceI, PleI, PmeI, PmlI, PpuMI, PshAI, PsiI, PspGI, PspOMI, PspXI, PstI, PstI-HF™, PvuI, PvuI-HF™, PvuII, PvuII-HF™, RsaI, RsrII, SacI, SacI-HF™, SacII, SalI, SalI-HF™, SapI, Sau3AI, Sau96I, SbfI, SbfI-HF™, ScaI, ScaI-HF™, ScrFI, SexAI, SfaNI, SfcI, SfiI, SfoI, SgrAI, SmaI, SmlI, SnaBI, SpeI, SphI, SphI-HF™, SspI, SspI-HF™, StuI, StyD4I, StyI, StyI-HF™, SwaI, TaqαI, TfiI, TliI, TscI, Tsp45I, Tsp509I, TspMI, TspRI, Tth111I, XbaI, XcmI, XhoI, XmaI, XmnI, and ZraI.

The one or more restriction enzymes used in the methods, compositions, and/or kits described herein can be derived from a variety of sources. For example, the one or more restriction enzymes can be produced from recombinant nucleic acid. The one or more restriction enzymes can be produced from recombinant nucleic acid in a heterologous host (e.g., in a bacteria, yeast, insect, or mammalian cell). The one or more restriction enzymes can be produced from recombinant nucleic acid in a heterologous host and purified from the heterologous host. The one or more restriction enzymes can be purified from a native source, e.g., a bacterium or archaea. If more than one restriction enzyme is used, at least one of the restriction enzymes can be from a recombinant source and at least one of the more than one restriction enzymes can be from a native source.

A recognition site for the one or more restriction enzymes can be any of a variety of sequences. For example, a recognition site for the one or more restriction enzymes can be a palindromic sequence. A recognition site for the one or more restriction enzymes can be a partially palindromic sequence. In some embodiments, a recognition site for the one or more restriction enzymes is not a palindromic sequence. A recognition site for the one or more restriction enzymes can be about, or more than, 1, 2, 3, 4, 5, 6, 7, 8, 9, 10, 11, 12, 13, 14, 15, 16, 17, 18, 19 or 20 bases or base pairs. A recognition site for a restriction enzyme can be about 2 to about 20, about 5 to about 20, about 5 to about 15, about 5 to about 10, about 7 to about 20, about 7 to about 15, or about 7 to about 10 bases or base pairs.

Two or more restriction enzymes can be used to digest a polynucleotide. The two or more restriction enzymes can recognize the same or different recognition sites. There can be one or more recognition sites for a single restriction enzyme between two target nucleic acid sequences on a single polynucleotide. There can be about, or at least, 1, 2, 3, 4, 5, 6, 7, 8, 9, 10 or more recognition sites for a single restriction enzyme between two target nucleic acid sequences on a single polynucleotide. There can be two or more different restriction enzyme recognition sites between two target nucleic acid sequences on a single polynucleotide. There can be about, or at least, 1, 2, 3, 4, 5, 6, 7, 8, 9, 10 or more different restriction enzyme recognition sites between two target nucleic acid sequences on a single polynucleotide. There can be one or more different restriction enzyme restriction sites between two target nucleic acid sequences on a single polynucleotide. There can be about, or at least, 1, 2, 3, 4, 5, 6, 7, 8, 9, 10 or more restriction enzyme restriction sites between two target nucleic acid sequences on a single polynucleotide.

A restriction enzyme digest can comprise one or more isoschizomer. Isoschizomers are restriction endonucleases that recognize the same sequence. The isoschizomers can have different cleavage sites; these enzymes are referred to as neoschizomers.

In some embodiments, cleavage by a restriction enzyme results in a blunt end. In some embodiments, cleavage by a restriction enzyme does not result in a blunt end. In some embodiments, cleavage by a restriction enzyme results in two fragments, each with a 5′ overhang. In some embodiments, cleavage by a restriction enzyme results in two fragments each with a 3′ overhang.

Primers for one or more amplification reactions can be designed to amplify sequences upstream and downstream of restriction enzyme cleavage site.

In one embodiment, a restriction enzyme does not cut the target nucleic acid sequence or a reference amplicon. One can use a reference sequence, e.g., a genome sequence, to predict whether a restriction enzyme will cut a nucleic acid sequence. In another embodiment, a restriction enzyme does cut the target nucleic acid sequence. The cleavage can occur near (within about 5, 10, 15, 25, 50. or 100 bp) of the 5′ or 3′ end of the target sequence, within the target sequence.

In another embodiment, a restriction enzyme does not cut the target or the reference nucleic acid sequence or amplicon even if the sequence or amplicon contains one or more SNPs. SNP information can be obtained from several databases, most readily from dbSNP (www.ncbi.nlm.nih.gov/projects/SNP/).

One or more methylation sensitive restriction enzymes can be used in the methods, compositions, and kits provided herein. The one or more methylation sensitive enzyme can include, e.g., DpnI, Acc65I, KpnI, ApaI, Bsp120I, Bsp143I, MboI, BspOI, NheI, Cfr9I, SmaI, Csp6I, RsaI, Ecl136II, SacI, EcoRII, MvaI, HpaII, or MspI. A methylation sensitive restriction enzyme cannot cleave a methylated nucleotide (e.g., cytocine) in a nucleic acid, but can cleave nucleic acid that is not methylated.

The restriction enzymes used in the present disclosure can be selected to specifically digest a selected region of nucleic acid sequence. The one or more restriction enzymes can cut between target nucleic acid sequences or target amplicons. One or more enzymes can be chosen whose recognition sequences occur e.g., once or multiple times—near the target nucleic acid sequences or target amplicons. Care can be taken to ensure that these recognition sequences are not affected by the presence of SNPs. In some cases, a recognition sequence of a restriction enzyme is not altered by a SNP.

A restriction enzyme can be an efficient but specific (no star activity) cutter. This property, along with digestion time and enzyme concentration, can be determined in advance by performing appropriate enzyme titration experiments. A restriction enzyme can have star activity. Star activity can be the cleavage of sequences that are similar but not identical to a defined recognition sequence.

The ratio of the number of “units” of a restriction enzyme to an amount of nucleic acid (e.g., DNA or RNA) can be, e.g., about, or at least, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 205, 210, 215, 220, 225, 230, 235, 240, 245, 250, 300, 350, 400, 500, 600, 700, 800, 900, 1000, 2000, 3000, 4000, 5000, 6000, 7000, 8000, 9000, 10,000, 12,000, 14,000, 15000, 16,000, 18,000, or 20,000 units/μg of nucleic acid. The ratio of the number of units of restriction enzyme to an amount of nucleic acid can be about 1 to about 20,000, about 1 to about 10,000, about 1 to about 5,000, about 100 to about 10,000, about 100 to about 1,000, about 50 to about 500, or about 50 to about 250 units/μg.

One or more restriction enzymes can be incubated with a sample comprising polynucleotides for about, or more than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes. One or more restriction enzymes can be incubated with a sample comprising polynucleotides for about, less than, at least, or more than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours. One or more restriction enzymes can be incubated with a sample comprising polynucleotides for about 1 to about 60 min., about 1 min. to about 48 hrs, about 1 min. to about 24 hrs, about 1 min. to about 20 hrs, about 1 min to about 16 hrs, about 0.5 hr to about 6 hrs, about 0.5 hr to about 3 hrs, about 1 hr to about 10 hrs, about 1 hr to about 5 hr, or about 1 hr to about 3 hr.

A restriction enzyme digest can be performed at a temperature of about, less than, at least, or more than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65° C. A restriction enzyme digest can be performed at a temperature of about 10 to about 65° C., about 20 to about 65° C., about 30 to about 65° C., about 37 to about 65° C., about 40 to about 65° C., about 50 to about 65° C., about 25 to about 37° C., about 25 to 30° C., about 30 to about 37° C., about 28 to 32° C., about 32 to 38° C., or about 35 to 38° C.

The pH of a restriction enzyme digest using one or more restriction enzymes can be about 2, 2.5, 3, 3.5, 4, 4.5, 5, 5.5, 6, 6.5, 6.6, 6.7, 6.8, 6.9, 7, 7.1, 7.2, 7.3, 7.4, 7.5, 7.5, 7.6, 7.7, 7.8, 7.9, 8, 8.1, 8.2, 8.3, 8.4, 8.5, 8.6, 8.7, 8.8, 8.9, 9, 9.5, 10, 10.5, 11, 11.5, 12, or 12.5. The pH of a restriction enzyme digest can be about 5 to about 9, about 5 to about 8, about 5 to about 7, about 6 to about 9, or about 6 to about 8.

A restriction enzyme digest can contain one or more buffers. The one or more buffers can be, e.g., tris-HCl, bis-tris-propane-HCl, TAPs, bicine, tris, tris-acetate, tris-HCl, tricine, TAPSO, HEPES, TES, MOPS, PIPES, cacodylate, SSC, phosphate buffer, collidine, veronal acetate, MES., ADA, ACES, cholamine chloride, acetamidoglycine, glycinamide, maleate, CABS, piperdine, glycine, citrate, glycylglycine, malate, formate, succinate, acetate, propionate, pyridine, piperazine, histidine, bis-tris, ethanolamine, carbonate, MOPSO, imidazole, BIS-TRIS propane, BES, MOBS, triethanolamine (TEA), HEPPSO, POPSO, hydrazine, Trizma (tris), EPPS, HEPPS, bicine, HEPBS, AMPSO, taurine (AES), borate, CHES, 2-amino-2-methyl-1-propanol (AMP), ammonium hydroxide, or methylamine. The concentration of a buffer in a solution can be, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. The concentration of buffer in a solution can be about 10 to about 100 mM, about 10 to about 75 mM, about 25 to about 75 mM, or about 10 to about 50 mM.

A restriction enzyme digest using one or more restriction enzymes can comprise bovine serum albumin (BSA). The concentration of BSA in a restriction digest can be about, less than, at least, or more than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 3, 4, 5, 6, 7, 8, 9, or 10 mg/ml. The concentration of BSA in a restriction digest can be about 0.01 to about 10 mg/ml, about 0.01 to about 1 mg/ml, about 0.05 to about 1 mg/ml, or about 0.05 to about 0.5 mg/ml.

A restriction enzyme digest using one or more restriction enzymes can comprise glycerol. Glycerol can be at a concentration (volume to volume) of about, less than, more than, or at least, 1, 2, 3, 4, 5, 6, 7, 8, 9, 10, 11, 12, 13, 14, 15, 16, 17, 18, 19, 20, 21, 22, 23, 24, or 25 percent. The concentration of glycerol in a restriction enzyme digest can be about 1 to about 25%, about 1 to about 20%, about 1 to about 15%, about 1 to about 10%, or about 1 to about 5%.

A restriction enzyme digest can comprise one or more organic solvents, e.g., DMSO, ethanol, ethylene glycol, dimethylacetamide, dimethylformamide, or suphalane. A restriction enzyme digest can be free of one or more organic solvents.

A restriction enzyme digest can comprise one or more divalent cations. The one or more divalent cations can be, e.g., Mg²⁺, Mn²⁺, Cu²⁺, Co²⁺, or Zn²⁺.

A restriction digest can comprise one or more salts. The one or more salts can include, for example, potassium acetate, potassium chloride, magnesium acetate, magnesium chloride, sodium acetate, or sodium chloride. The concentration of each of the one or more salts can be, e.g., about, less than, at least, or more than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 205, 210, 215, 220, 225, 230, 235, 240, 245, or 250 mM. The concentration of each of the one or more salts can be about 5 to about 250, about 5 to about 200, about 5 to about 150, about 5 to about 100, about 10 to about 100, about 10 to about 90, about 10 to about 80, about 10 to about 70, about 10 to about 60, or about 10 to about 50 mM.

A restriction digest can comprise one or more reducing agents. The one or more reducing agents can inhibit the formation of disulfide bonds in a protein. A reducing agent can be, for example, dithiothreitol (DTT), 2-mercaptoethanol (BME), 2-mercaptoethylamine-HCl, tris(2-carboxythyl)phosphine (TCEP), or cysteine-HCl, The concentration of the one or more reducing agents in a restriction enzyme digest can be about, less than, at least, or more than, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, or 25 mM. The concentration of the one or more reducing agents in a restriction enzyme digest can be about 0.01 to about 25 mM, about 0.01 to about 15 mM, about 0.01 to about 10 mM, about 0.01 to about 5 mM, about 0.1 to about 5 mM, or about 0.5 to about 2.5 mM.

More than one restriction enzyme can be used in a restriction enzyme digest of nucleic acid. For example, multiple-digests can be employed if one or more of the restriction enzymes do not efficiently cut a nucleic acid, or if they do not all work universally well across all samples (e.g., because of SNPs). Multiple-digets by the one or more restriction enzymes can be performed simultaneously in the same reaction solution, or serially (e.g., add one restriction enzyme, purify the nucleic acid after the first digest, and add another restriction enzyme). The number of different restriction enzymes that can be used in a restriction digest can be about, or at least, 1, 2, 3, 4, 5, 6, 7, 8, 9, 10, 11, 12, 13, 14, 15, 16, 17, 18, 19, or 20. The number of different restriction enzymes that can be used in a restriction enzyme digest can be, e.g., about 1 to about 20, about 1 to about 15, about 1 to about 10, about 1 to about 7, about 1 to about 6, about 1 to about 5, about 1 to about 4, about 1 to about 3, or about 1 to about 2.

In some cases, PCR works better when the size of the fragment containing the amplicon or targets is relatively small. Therefore, selecting restriction enzymes with cutting sites near the amplicons or target can be desirable. For example, a restriction enzyme recognition site or cleavage site can be within about, or less than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 205, 210, 215, 220, 225, 230, 235, 240, 245, 250, 300, 350, 400, 500, 600, 700, 800, 900, 1000, 2000, 3000, 4000, 5000, 6000, 7000, 8000, 9000, 10,000, 12,000, 14,000, 15000, 16,000, 18,000, or 20,000 base pairs from the 5′ end or 3′ end of one of the targets on a polynucleotide. A restriction enzyme recognition site or cleavage site can be within about 1 to about 10,000, about 1 to about 5,000, about 1 to about 2,500, about 1 to about 1,000, about 1 to about 100, about 100 to about 1000, about 100 to about 500, or about 100 to about 250 bp from the 5′ or 3′ end of a target nucleic acid sequence.

A single sample can be analyzed for multiple CNVs. In this case, it can be desirable to select the smallest number of digests that would work well for the entire set of CNVs. A single restriction enzyme cocktail can be found that does not cut within any of the amplicons or target nucleic acid sequences but has recognition sites or cleavage sites near each one of them. A restriction enzyme recognition site or cleavage site can be within about, or less than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 205, 210, 215, 220, 225, 230, 235, 240, 245, 250, 300, 350, 400, 500, 600, 700, 800, 900, 1000, 2000, 3000, 4000, 5000, 6000, 7000, 8000, 9000, 10,000, 12,000, 14,000, 15000, 16,000, 18,000, or 20.000 base pairs from the 5′ end or 3′ end of one of the targets on a polynucleotide. A restriction enzyme recognition site or cleavage site can be within about 1 to about 20,000, about 10 to about 20,000, about 100 to about 20,000, about 1000 to about 20,000, about 10 to about 10,000, about 10 to about 1000, about 10 to about 100, about 50 to about 20,000, about 50 to about 1000, about 50 to about 500. about 50 to about 250. about 50 to about 150. or about 50 to 100 base pairs from the 5′ end or 3′ end of one or the targets on a polynucleotide.

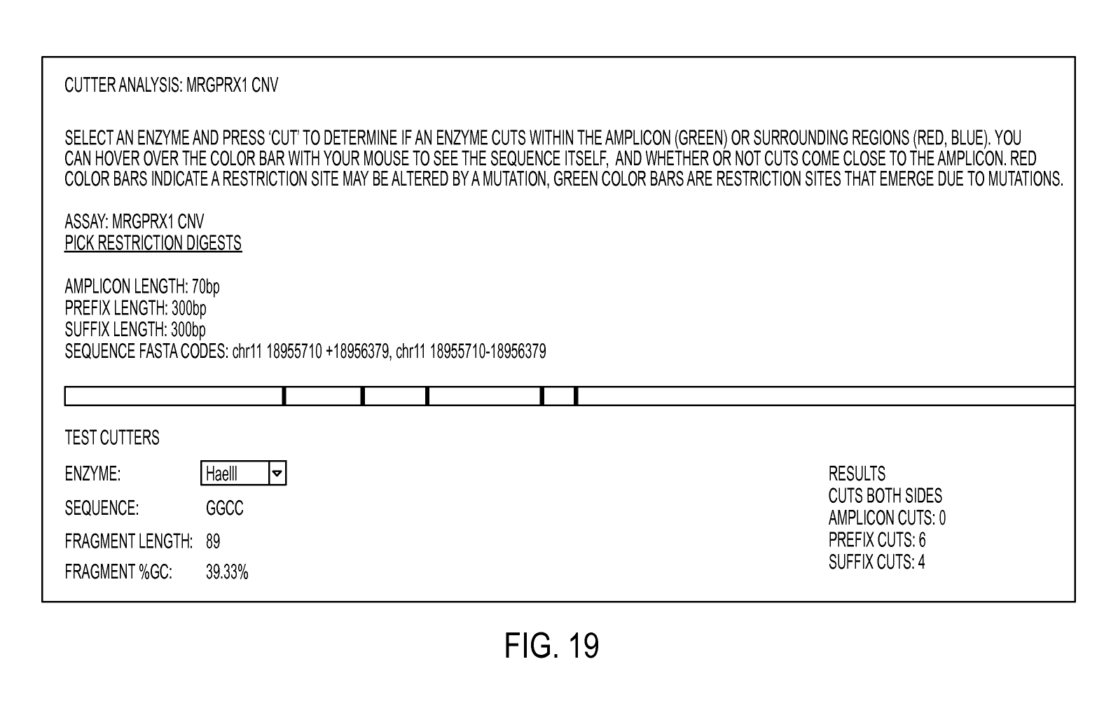

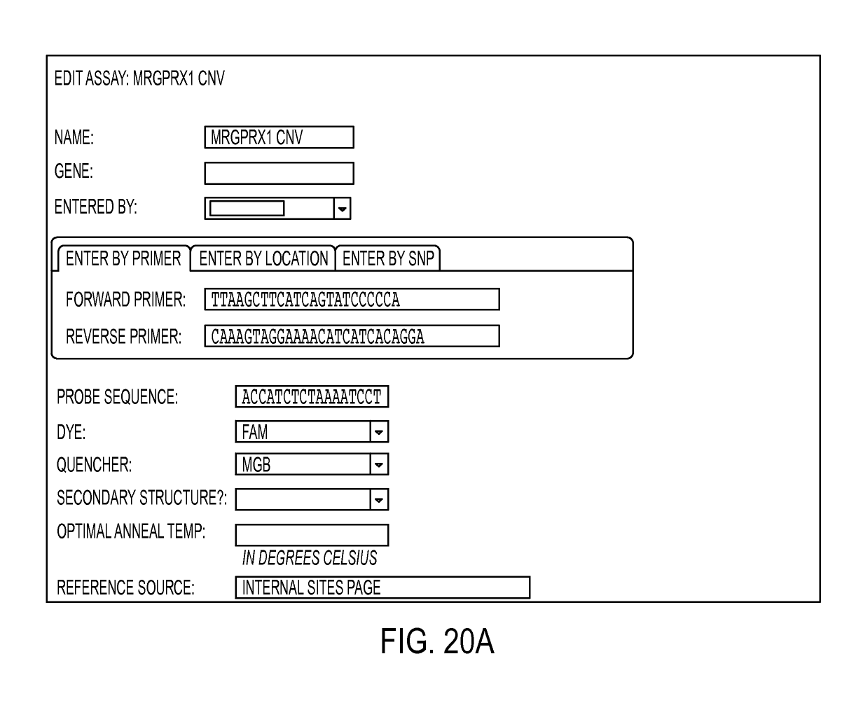

Appropriate software can be written and/or used to automate the process of restriction enzyme choice and present an interface for a user, e.g., an experimental biologist, to choose the most appropriate enzymes given the criteria above. Additional considerations can be employed by the software, such as enzyme cost, enzyme efficiency, buffer compatibility of restriction enzymes, reaction conditions (e.g., temperature, time, etc.), methylation sensitivity, number of cleavage sites in a segment of nucleic acid, or availability. The software can be used on a computer. An algorithm can be generated on a computer readable medium and be used to select one or more restriction enzymes for digesting nucleic acid. A computer can be connected to the internet and can be used to access a website that can permit selection of restriction endonucleases. A web tool can be used to select restriction enzymes that will cut around an amplicon in order to separate linked gene copies for CNV estimation. For example, enzymes and assays can be stored in a database and selection of a restriction enzyme can be automatic. Additional statistics that can be considered include, e.g., length of shortest fragment, % GC content, frequency of cuts around (or in) an amplicon, and cost of enzymes. QTools can be used to assist in the selection of one or more restriction enzymes. FIGS. 18 and 19 illustrate information that can be considered when selecting a restriction enzyme.

For assay storage for data analysis, a researcher can enter assays by location or primer sequence. QTools can automatically retrieve and stores amplicon sequences and known SNPs and compute thermodynamic parameters. As researchers use the assay more, they can enter additional data, including confirmed sample CNVs and annealing temperatures.

Digestion with more than one enzyme, performed serially or together in a single tube, can help to ensure complete cutting of difficult targets. A series of restriction enzyme digests of one sample can be performed with different enzymes, e.g., about 1, 2, 3, 4, 5, 6, 7, 8, 9, or 10. In some cases, serial digests can include purifying the sample before adding the next restriction enzyme.

One or more restriction enzymes in a digest can be inactivated following the restriction enzyme digest. In some embodiments, the one or more restriction enzymes cannot be inactivated by exposure to heat. Most restriction enzymes can be heat-inactivated after restriction by raising the temperature of the restriction reaction. The temperature for heat-inactivation can be, e.g., about, less than, at least, or more than, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C. The temperature for heat inactivation can be about 50 to about 100, about 50 to about 90, about 60 to about 90, about 65 to about 90, about 65 to about 85, or about 65 to about 80° C. The duration of heat-inactivation can be, e.g., about, less than, at least, or more than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 80, 90, 100, 110, 120, 180, 240, 300, 360, 420, 480, 540, 600, 660 or 720 minutes. The duration of heat-inactivation can be about 5 to about 300, about 5 to about 200, about 5 to about 150, about 5 to about 100, about 5 to about 75, about 5 to about 50, about 5 to about 40, about 5 to about 30, about 5 to about 35, about 5 to about 25, about 5 to about 20, or about 10 to about 20 minutes. The temperature of heat-inactivation can be below the melt point of the restricted target fragments, so as to maintain double-stranded template copies.

A restriction enzyme digest can be stopped by addition of one or more chelating agents to the restriction enzyme digest. The one or more chelating agents can be, e.g., EDTA, EGTA, citric acid, or a phosphonate. The concentration of the one or more chelating agents in a restriction enzyme digest can be, e.g., about, or at least, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. The concentration of the one or more chelating agents can be about 1 to about 100 mM, about 1 to about 75 mM, or about 25 to about 75 mM.

A control assay and template can be used to measure the efficiency of a restriction enzyme digestion step.

Samples

Samples to be analyzed using the methods, compositions, and kits provided herein can be derived from a non-cellular entity comprising nucleic acid (e.g., a virus) or from a cell-based organism (e.g., member of archaea, bacteria, or eukarya domains). A sample can be obtained in some cases from a hospital, laboratory, clinical or medical laboratory. The sample can comprise nucleic acid, e.g., RNA or DNA. The sample can comprise cell-free nucleic acid. In some cases, the sample is obtained from a swab of a surface, such as a door or bench top.

The sample can be from a subject, e.g., a plant, fungi, eubacteria, archeabacteria, protest, or animal. The subject can be an organism, either a single-celled or multi-cellular organism. The subject may be cultured cells, which can be primary cells or cells from an established cell line, among others. The sample can be isolated initially from a multi-cellular organism in any suitable form. The animal can be a fish, e.g., a zebrafish. The animal can be a mammal. The mammal can be, e.g., a dog, cat, horse, cow, mouse, rat, rabbit, or pig. The mammal can be a primate, e.g., a human, chimpanzee, orangutan, monkey, or gorilla. The human can be a male or female. The sample can be from a human embryo or human fetus. The human can be an infant, child, teenager, adult, or elderly person. The female can be pregnant, can be suspected of being pregnant, or planning to become pregnant.

The sample can be from a subject (e.g., human subject) who is healthy. In some embodiments, the sample is taken from a subject (e.g., an expectant mother) at at least 4, 5, 6, 7, 8, 9, 10, 11, 12, 13, 14, 15, 16, 17, 18, 19, 20, 21, 22, 23, 24, 25, or 26 weeks of gestation. The subject can be affected by a genetic disease, a carrier for a genetic disease or at risk for developing or passing down a genetic disease, where a genetic disease is any disease that can be linked to a genetic variation such as mutations, insertions, additions, deletions, translocation, point mutation, trinucleotide repeat disorders and/or single nucleotide polymorphisms (SNPs). A sample can be taken from a female patient of child-bearing age and, in some cases, the female patient is not pregnant or of unknown pregnancy status. The subject can be a male patient, a male expectant father, or a male patient at risk of, diagnosed with, or having a specific genetic abnormality. In some cases, a female patient is known to be affected by, or is a carrier of, a genetic disease or genetic variation, or is at risk of, diagnosed with, or has a specific genetic abnormality. In some cases, the status of the female patient with respect to a genetic disease or genetic variation may not be known. A sample can be taken from any child or adult patient of known or unknown status with respect to copy number variation of a genetic sequence. In some cases, the child or adult patient is known to be affected by, or is a carrier of, a genetic disease or genetic variation. In some cases, a sample is from a subject with a neurological condition. In some cases, a sample is from a subject at risk or suspected of having a neurological condition. The neurological condition can be Alzheimer's disease, autism, or schizophrenia.

The sample can be from a subject who has a specific disease, disorder, or condition, or is suspected of having (or at risk of having) a specific disease, disorder or condition. For example, the sample can be from a cancer patient, a patient suspected of having cancer, or a patient at risk of having cancer. The cancer can be, e.g., acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloeptithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chromic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, nonmelanoma, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom Macroglobulinemia, or Wilms Tumor. The sample can be from the cancer and/or normal tissue from the cancer patient.

In some cases, the sample can be from a pregnant female whose fetus has, is suspected of having, or at risk of having aneuploidies. The sample can be from the fetus, the pregnant female or both. The sample can comprise genomic DNA or cell-free DNA.

The sample can be from a subject who is known to have a genetic disease, disorder or condition. In some cases, the subject is known to be wild-type or mutant for a gene, or portion of a gene, e.g., CFTR, Factor VIII (F8 gene), beta globin, hemachromatosis, G6PD, neurofibromatosis, GAPDH, beta amyloid, or pyruvate kinase gene. In some cases, the status of the subject is either known or not known, and the subject is tested for the presence of a mutation or genetic variation of a gene, e.g., CFTR, Factor VIII (F8 gene), beta globin, hemachromatosis, G6PD, neurofibromatosis, GAPDH, beta amyloid, or pyruvate kinase gene.

The sample can be aqueous humour, vitreous humour, bile, whole blood, blood serum, blood plasma, breast milk, cerebrospinal fluid, cerumen, enolymph, perilymph, gastric juice, mucus, peritoneal fluid, saliva, sebum, semen, sweat, tears, vaginal secretion, vomit, feces, or urine. The sample can be obtained from a hospital, laboratory, clinical or medical laboratory. The sample can be taken from a subject. The sample can comprise nucleic acid. The nucleic acid can be, e.g., mitochondrial DNA, genomic DNA, mRNA, siRNA, miRNA, cRNA, single-stranded DNA, double-stranded DNA, single-stranded RNA, double-stranded RNA, tRNA, rRNA, or cDNA. The sample can comprise cell-free nucleic acid. The sample can be a cell line, genomic DNA, cell-free plasma, formalin fixed paraffin embedded (FFPE) sample, or flash frozen sample. A formalin fixed paraffin embedded sample can be deparaffinized before nucleic acid is extracted. The sample can be from an organ, e.g., heart, skin, liver, lung, breast, stomach, pancreas, bladder, colon, gall bladder, brain, etc.

When the nucleic acid is RNA, the source of the RNA can be any source described herein. For example, the RNA can a cell-free mRNA, can be from a tissue biopsy, core biopsy, fine needle aspirate, flash frozen, or formalin-fixed paraffin embedded (FFPE) sample. The FFPE sample can be deparaffinized before the RNA is extracted. The extracted RNA can be heated to about 30, 31, 32, 33, 34, 35, 36, 37 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99° C. before analysis. The extracted RNA can be heated to any these temperatures for about, more than, less than, or at least, 15 min, 30 min, 45 min, 60 min, 1.5 hr, 2 hr, 2.5 hr, 3 hr, 3.5 hr, 4 hr, 4.5 hr, 5 hr, 5.5 hr, 6 hr, 6.5 hr, 7 hr, 7.5 hr, 8 hr, 8.5 hr, 9 hr, 9.5 hr, or 10 hr.

RNA can be used for a variety of downstream applications. For example, the RNA can be converted to cDNA with a reverse transcriptase and the cDNA can optionally be subject to PCR, e.g., real-time PCR or quantative PCR. The RNA or cDNA can be used in an isothermal amplification reaction, e.g., an isothermal linear amplification reaction. The RNA, resulting cDNA, or molecules amplified therefrom can be used in a microarray experiment, gene expression experiment, Northern analysis, Southern analysis, sequencing reaction, next generation sequencing reaction, etc. Specific RNA sequences can be analyzed, or RNA sequences can be globally analyzed.

Nucleic acids can be extracted from a sample by means available to one of ordinary skill in the art. For example, nucleic acids can be extracted by precipitation using organic solvents (e.g., ethanol or isopropanol), or a DNA-binding spin column (e.g., Qiagen DNA mini kit).

A sample can be processed to render it competent for amplification. Exemplary sample processing can include lysing cells of the sample to release nucleic acid, purifying the sample (e.g., to isolate nucleic acid from other sample components, which may inhibit amplification), diluting/concentrating the sample, and/or combining the sample with reagents for amplification, such as a DNA/RNA polymerase (e.g., a heat-stable DNA polymerase for PCR amplification), dNTPs (e.g., dATP, dCTP, dGTP, and dTTP (and/or dUTP)), a primer set for each allele sequence or polymorphic locus to be amplified, probes (e.g., fluorescent probes, such as TAQMAN probes or molecular beacon probes, among others) capable of hybridizing specifically to each allele sequence to be amplified, Mg²⁺, DMSO, BSA, a buffer, or any combination thereof, among others. In some examples, a sample may be combined with a restriction enzyme, uracil-DNA glycosylase (UNG), reverse transcriptase, or any other enzyme of nucleic acid processing.

Target Polynucleotide

The term polynucleotide, or grammatical equivalents, can refer to at least two nucleotides covalently linked together. A nucleic acid described herein can contain phosphodiester bonds, although in some cases, as outlined below (for example in the construction of primers and probes such as label probes), nucleic acid analogs are included that can have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid (also referred to herein as “PNA”) backbones and linkages (see Egholm, J. Am Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids (also referred to herein as “LNA”). Koshkin et al., J. Am. Chem. Soc. 120.13252 3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & amp; Nucleotide 13:1597 (1994); Chapters 2 and 3. ASC Symposium Series 580. “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580. “Carbohydrate Modifications in Antisense Research”, Ed. Y. S. Sanghui and P Dan Cook Nucleic acids containing one or more carbocyclic sugars are also included within the definition of nucleic acids (see Jenkins et al., Chem. Soc. Rev. (1995) pp 169 176). Several nucleic acid analogs are described in Rawls, C & E News Jun. 2, 1997 page 35 “Locked nucleic acids” are also included within the definition of nucleic acid analogs. LNAs are a class of nucleic acid analogues in which the ribose ring is “locked” by a methylene bridge connecting the 2′-O atom with the 4′-C atom. All of these references are hereby expressly incorporated by reference. These modifications of the ribose-phosphate backbone can be done to increase the stability and half-life of such molecules in physiological environments. For example, PNA:DNA and LNA-DNA hybrids can exhibit higher stability and thus can be used in some embodiments. The target nucleic acids can be single stranded or double stranded, as specified, or contain portions of both double stranded or single stranded sequence. Depending on the application, the nucleic acids can be DNA (including, e.g., genomic DNA, mitochondrial DNA, and cDNA), RNA (including, e.g., mRNA and rRNA) or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

The methods and compositions provided herein can be used to evaluate a quantity of polynucleotides (e.g., DNA, RNA, mitochondrial DNA, genomic DNA, mRNA, siRNA, miRNA, cRNA, single-stranded DNA, double-stranded DNA, single-stranded RNA, double-stranded RNA, tRNA, rRNA, cDNA, etc.). The methods and compositions can be used to evaluate a quantity of a first polynucleotide compared to the quantity of a second polynucleotide. The methods can be used to analyze the quantity of synthetic plasmids in a solution; to detect a pathogenic organism (e.g., microbe, bacteria, virus, parasite, retrovirus, lentivirus, HIV-1, HIV-2, influenza virus, etc.) within a sample obtained from a subject or obtained from an environment. The methods also can be used in other applications wherein a rare population of polynucleotides exists within a larger population of polynucleotides.

The number of copies of a target nucleic acid sequence in a sample (e.g., a genome) of a subject whose sample is analyzed using the methods, compositions, and kits provided herein can be 0, or about, more than, less than, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1000, 5000, 10,000, 20,000, 50,000, or 100,000. The number of copies of a target nucleic acid sequence in a genome of a subject whose sample is analyzed using the methods, compositions, and kits provided herein can be about 1 to about 20, about 1 to about 15, about 1 to about 10, about 1 to about 7, about 1 to about 5, about 1 to about 3, about 1 to about 1000, about 1 to about 500, about 1 to about 250, about 1 to about 100, about 10 to about 1000, about 10 to about 500, about 10 to about 250, about 10 to about 100, about 10 to about 50, about 10 to about 20, about 0 to about 100, about 0 to about 50, about 0 to about 25 or about 0 to about 10.

The target nucleic acid sequence can be on one chromosome. If the target nucleic acid is in a sample derived from a human subject, the target nucleic acid sequence can be on one or more of chromosome 1, 2, 3, 4, 5, 6, 7, 8, 9, 10, 11, 12, 13, 14, 15, 16, 17, 18, 19, 20, 21, 22, X, or Y. The target nucleic acid can be on about, at least, less than, or more than, 1, 2, 3, 4, 5, 6, 7, 8, 9, 10, 11, 12, 13, 14, 15, 16, 17, 18, 19, 20, 21, 22, or 23 chromosomes. Two or more copies of the target nucleic acid sequence can be on the same or different chromosomes. In a human subject, two or more copies of the target nucleic acid sequence can be on chromosome 1, 2, 3, 4, 5, 6, 7, 8, 9, 10, 11, 12, 13, 14, 15, 16, 17, 18, 19, 20, 21, 22, X, or Y. Two or more copies of the target nucleic acid sequence can be on one polynucleotide (e.g., chromosome) in a subject, but the target nucleic acids can be separated in a sample taken from the subject due to handling of the sample (e.g., by fragmentation).

When two copies of a target nucleic acid are on the same polynucleotide, e.g., same chromosome, the two copies can be spaced apart on the polynucleotide by about, at least, more than, or less than 1, 2, 3, 4, 5, 6, 7, 8, 9, 10, 25, 50, 75, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1 million, 2 million, 3 million, 4 million, 5 million, 6 million, 7 million, 8 million, 9 million, 10 million, 20 million, 30 million, 40 million, 50 million, 60 million, 70 million, 80 million, 90 million, or 100 million bases or base pairs. Two target nucleic acids can be spaced apart by about 100 to about 100,000, about 100 to about 10,000, about 100 to about 1,000, about 10 to about 10,000, or about 10 to about 1,000 bases or base pairs.

The target sequence can be a gene. For example, the gene can be ERBB2, EGFR, BRCA1, BRCA2, APC, MSH2, MSH6, MLH1, CYP2D6, a low-copy repeat (LCR)-rich sequence (see e.g., Balikova et al. (2008) Am J. Hum Genet. 82: 181-187), TAS1R1, GNAT1, IMPDH1, OPN1SW, OR2A12, OR2A14, OR2A2, OR2A25, OR2A5, OR2A1, OR2A42, OR2A7, OR4F21, OR4F29, OR4C6, OR4P4, OR4S2, OR5D13, ROM1, TAS@R14, TAS2R44, TAS2R48, TAS2R49, TAS2R50, OR6C2, OR6C4, OR6C68, OR6C70, OR4MI, OR4Q3, OR4K1, OR4K2, OR4K5, OR4N2, OR4K13, OR4K14, OR4K15, OR4M2, OR4N4, OR1F1, ACTG1, FSCN2, OR2Z1, OR11H1, MYH9, SKI, TP73, TNFRSF25, RAB3B, VAV3, RALB, BOK, NAT6, TUSC2, TUSC4, TAB33B, C6orf210, ESR1, MAFK, MAD1L1, MYC, VAV2, MAP3K8, CDKN1C, WT1, WIT-1, C1QTNF4, MEN1, CCND1, ORAOV1, MLL2, C13orf10, TNFAIP2, AXIN1, BCAR1, TAX1BP3, NF1, PHB, MAFG, C1QTNF1, YES1, DCC, SH3GL1, TNFSF9, TNFSF7, TNFSF14, VAV1, RAB3A, PTOV1, BAX, RRAS, BCAS4, HIC2, NROB2, TTN, SGCB, SMA3, SMA4, SMN1, LPA, PARK2, GCK, GPR51, BSCL2, A2M, TBXA2R, FKRP, or COMT.

The target sequence can encode a microRNA, e.g., hsa-let-7g, hsa-mir-135a-1, hsa-mir-95, hsa-mir-218-1, hsa-mir-320, has-let-7a-1, has-let-7d, has-let-7f-1, has-mir-202, has-mir-130a, has-mir-130a, has-mir-338, has-mir-199a-1, has-mir-181c, has-mir-181d, has-mir-23a, has-mir-24-2, has-mir-27a, has-mir-150, has-mir-499, has-mir-124a-3, or has-mir-185.

The target sequence can be any sequence listed in Wong et al. (2007) Am J of Hum Genetics 80: 91-104.

Amplification and Detection

The methods described herein can make use of nucleic acid amplification. Amplification of target nucleic acids can be performed by any means known in the art. Amplification can be performed by thermal cycling or isothermally. In exemplary embodiments, amplification may be achieved by the polymerase chain reaction (PCR).

Examples of PCR techniques that can be used include, but are not limited to, quantitative PCR, quantitative fluorescent PCR (QF-PCR), multiplex fluorescent PCR (MF-PCR), real time PCR(RT-PCR), single cell PCR, restriction fragment length polymorphism PCR (PCR-RFLP), PCR-RFLP/RT-PCR-RFLP, hot start PCR, nested PCR, in situ polony PCR, in situ rolling circle amplification (RCA), bridge PCR, picotiter PCR, digital PCR, droplet digital PCR, and emulsion PCR. Other suitable amplification methods include the ligase chain reaction (LCR), transcription amplification, molecular inversion probe (MIP) PCR, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938. Amplification of target nucleic acids can occur on a bead. In other embodiments, amplification does not occur on a bead. Amplification can be by isothermal amplification, e.g., isothermal linear amplification. A hot start PCR can be performed wherein the reaction is heated to 95° C. for two minutes prior to addition of the polymerase or the polymerase can be kept inactive until the first heating step in cycle 1. Hot start PCR can be used to minimize nonspecific amplification. Other strategies for and aspects of amplification are described in U.S. Patent Application Publication No. 2010/0173394 A1, published Jul. 8, 2010, which is incorporated herein by reference.

Techniques for amplification of target and reference sequences are known in the art and include the methods described in U.S. Pat. No. 7,048,481. Briefly, the techniques can include methods and compositions that separate samples into small droplets, in some instances with each containing on average less than 5, 4, 3, 2, or one target nucleic acid molecule (polynucleotide) per droplet, amplifying the nucleic acid sequence in each droplet and detecting the presence of a target nucleic acid sequence. In some cases, the sequence that is amplified is present on a probe to the genomic DNA, rather than the genomic DNA itself. In some cases, at least 200, 175, 150, 125, 100, 90, 80, 70, 60, 50, 40, 30, 20, 10, or 0 droplets have zero copies of a target nucleic acid.

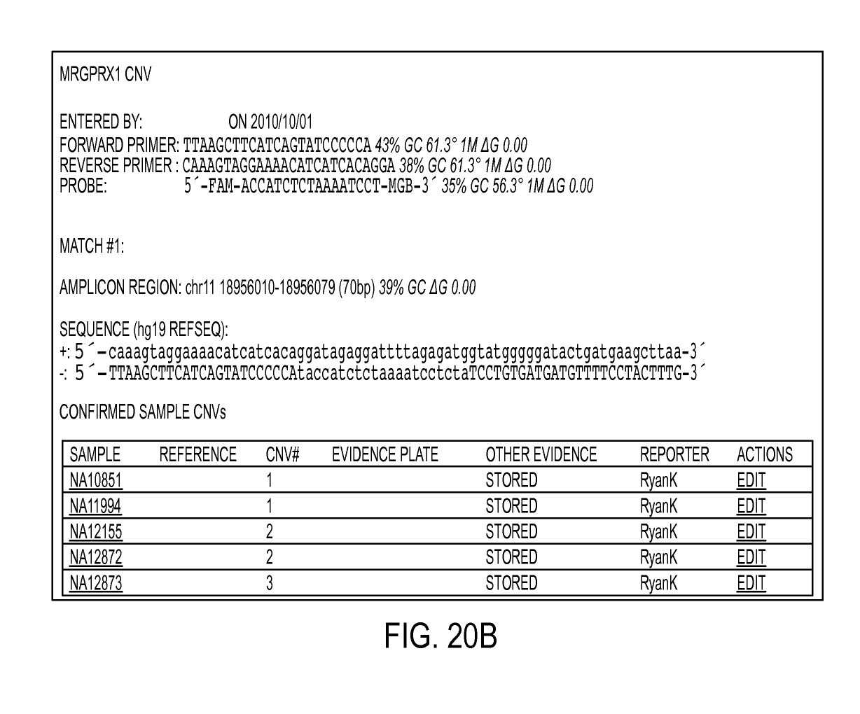

Information about an amplification reaction can be entered into a database. For example, FIGS. 20A and 20B illustrate assay information that can be entered into a database.

Primers

Primers can be designed according to known parameters for avoiding secondary structures and self-hybridization. Different primer pairs can anneal and melt at about the same temperatures, for example, within about 1, 2, 3, 4, 5, 6, 7, 8, 9 or 10° C. of another primer pair. In some cases, greater than about 1, 2, 3, 4, 5, 6, 7, 8, 9, 10, 15, 20, 25, 30, 35, 40, 45, 50, 100, 200, 500, 1000, 5000, 10,000 or more primers are initially used. Such primers may be able to hybridize to the genetic targets described herein. In some cases, about 2 to about 10,000, about 2 to about 5,000, about 2 to about 2,500, about 2 to about 1,000, about 2 to about 500, about 2 to about 100, about 2 to about 50, about 2 to about 20, about 2 to about 10, or about 2 to about 6 primers are used.

Primers can be prepared by a variety of methods including but not limited to cloning of appropriate sequences and direct chemical synthesis using methods well known in the art (Narang et al., Methods Enzymol. 68:90 (1979); Brown et al., Methods Enzymol. 68:109 (1979)). Primers can also be obtained from commercial sources such as Integrated DNA Technologies, Operon Technologies, Amersham Pharmacia Biotech, Sigma, and Life Technologies. The primers can have an identical melting temperature. The melting temperature of a primer can be about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 81, 82, 83, 84, or 85° C. In some embodiments, the melting temperature of the primer is about 30 to about 85° C., about 30 to about 80° C., about 30 to about 75° C., about 30 to about 70° C., about 30 to about 65° C., about 30 to about 60° C., about 30 to about 55° C., about 30 to about 50° C., about 40 to about 85° C., about 40 to about 80° C., about 40 to about 75° C., about 40 to about 70° C., about 40 to about 65° C., about 40 to about 60° C., about 40 to about 55° C., about 40 to about 50° C., about 50 to about 85° C., about 50 to about 80° C., about 50 to about 75° C., about 50 to about 70° C., about 50 to about 65° C., about 50 to about 60° C., about 50 to about 55° C., about 52 to about 60° C., about 52 to about 58° C., about 52 to about 56° C., or about 52 to about 54° C.

The lengths of the primers can be extended or shortened at the 5′ end or the 3′ end to produce primers with desired melting temperatures. One of the primers of a primer pair can be longer than the other primer. The 3′ annealing lengths of the primers, within a primer pair, can differ. Also, the annealing position of each primer pair can be designed such that the sequence and length of the primer pairs yield the desired melting temperature. An equation for determining the melting temperature of primers smaller than 25 base pairs is the Wallace Rule (Td=2(A+T)+4(G+C)). Computer programs can also be used to design primers, including but not limited to Array Designer Software (Arrayit Inc.), Oligonucleotide Probe Sequence Design Software for Genetic Analysis (Olympus Optical Co.), NetPrimer, and DNAs is from Hitachi Software Engineering. The TM (melting or annealing temperature) of each primer can be calculated using software programs such as Net Primer (free web based program at http://www.premierbiosoft.com/netprimer/index.html). The annealing temperature of the primers can be recalculated and increased after any cycle of amplification, including but not limited to about cycle 1, 2, 3, 4, 5, about cycle 6 to about cycle 10, about cycle 10 to about cycle 15, about cycle 15 to about cycle 20, about cycle 20 to about cycle 25, about cycle 25 to about cycle 30, about cycle 30 to about cycle 35, or about cycle 35 to about cycle 40. After the initial cycles of amplification, the 5′ half of the primers can be incorporated into the products from each loci of interest; thus the TM can be recalculated based on both the sequences of the 5′ half and the 3′ half of each primer.

The annealing temperature of the primers can be recalculated and increased after any cycle of amplification, including but not limited to about cycle 1, 2, 3, 4, 5, about cycle 6 to about cycle 10, about cycle 10 to about cycle 15, about cycle 15 to about cycle 20, about cycle 20 to about cycle 25, about cycle 25 to about cycle 30, about cycle 30 to about 35, or about cycle 35 to about cycle 40. After the initial cycles of amplification, the 5′ half of the primers can be incorporated into the products from each loci of interest, thus the TM can be recalculated based on both the sequences of the 5′ half and the 3′ half of each primer.

DNA Polymerase

Any DNA polymerase that catalyzes primer extension can be used including but not limited to E. coli DNA polymerase, Klenow fragment of E. coli DNA polymerase 1, T7 DNA polymerase, T4 DNA polymerase, Taq polymerase, Pfu DNA polymerase, Pfx DNA polymerase, Tth DNA polymerase, Vent DNA polymerase, bacteriophage 29. REDTaq™, Genomic DNA polymerase, or sequenase. A thermostable DNA polymerase can be used. The DNA polymeras can have 3′ to 5′ exonuclease activity the DNA polymeras can possess 5′ to 3′ exonuclease activity. The DNA polymerase can possess both 3′ to 5′ exonuclease activity and 5′ to 3′ exonuclease activity. In some cases, the DNA polymerase has strand displacement activity. In some cases, the DNA polymerase does not have strand displacement activity. In some cases, the DNA polymerase has weak strand displacement activity. In some cases, the DNA polymerase has strong strand displacement activity.

Thermocycling

Any number of PCR cycles can be used to amplify DNA, e.g., about, at least, more than, or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 cycles. The number of amplification cycles can be about 1 to about 45, about 10 to about 45, about 20 to about 45, about 30 to about 45, about 35 to about 45, about 10 to about 40, about 10 to about 30, about 10 to about 25, about 10 to about 20, about 10 to about 15, about 20 to about 35, about 25 to about 35, about 30 to about 35, or about 35 to about 40.

Thermocycling reactions can be performed on samples contained in droplets. The droplets can remain intact during thermocycling. Droplets can remain intact during thermocycling at densities of greater than about 10,000 droplets/mL, 100,000 droplets/mL, 200,000 droplets/mL, 300,000 droplets/mL, 400,000 droplets/mL, 500,000 droplets/mL, 600,000 droplets/mL, 700,000 droplets/mL, 800,000 droplets/mL, 900,000 droplets/mL or 1,000,000 droplets/mL. In other cases, two or more droplets may coalesce during thermocycling. In other cases, greater than 100 or greater than 1,000 droplets may coalesce during thermocycling.

Probes

Universal probes can be designed by methods known in the art. In some cases, a probe comprises a random sequence. A universal probe can be selected to ensure that it does not bind the target polynucleotide in an assay, or to other non-target polynucleotides likely to be in a sample (e.g., genomic DNA outside the region occupied by the target polynucleotide).

A label (fluorophore, dye) used on a probe (e.g., a Taqman probe) to detect a target nucleic acid sequence or reference nucleic acid sequence in the methods described herein can be, e.g., 6-carboxyfluorsecin (FAM), tetrachlorofluorescein (TET), 4,7,2′-trichloro-7′-phenyl-6-carboxyfluorescein (VIC), HEX, Cy3, Cy 3.5, Cy 5, Cy 5.5, Cy 7, tetramethylrhodamine, ROX, and JOE. The label can be an Alexa Fluor dye, e.g., Alexa Fluor 350, 405, 430, 488, 532, 546, 555, 568, 594, 633, 647, 660, 680, 700, and 750. The label can be Cascade Blue, Marina Blue, Oregon Green 500, Oregon Green 514, Oregon Green 488, Oregon Green 488-X, Pacific Blue, Rhodamine Green, Rhodol Green, Rhodamine Green-X, Rhodamine Red-X, and Texas Red-X. The label can be at the 5′ end of a probe, 3′ end of the probe, at both the 5′ and 3′ end of a probe, or internal to the probe. A unique label can be used to detect each different locus in an experiment.

A probe, e.g., a Taqman probe, can comprise a quencher, e.g., a 3′ quencher. The 3′ quencher can be, e.g., TAMARA, DABCYL, BHQ-1, BHQ-2, or BHQ-3. In some cases, a quencher used in the methods provided herein is a black hole quencher (BHQ). In some cases, the quencher is a minor groove binder (MGB). In some cases, the quencher is a fluorescent quencher. In other cases, the quencher is a non-fluorescent quencher (NFQ).

A probe can be about, more than, less than, or at least, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases long. A probe can be about 8 to about 40, about 10 to about 40, about 10 to about 35, about 10 to about 30, about 10 to about 25, about 10 to about 20, about 15 to about 40, about 15 to about 35, about 15 to about 30, about 15 to about 25, about 15 to about 20, about 18 to about 40, about 18 to about 35, about 18 to about 30, about 18 to about 25, or about 18 to 22 bases.

Reagents and Additives

Solution and reagents for performing a PCR reaction can include buffers. The buffered solution can comprise about, more than, at least, or less than 1, 5, 10, 15, 20, 30, 50, 100, or 200 mM Tris. In some cases, the solution and reagents comprise potassium chloride (KCl). The concentration of potassium chloride can be about, more than, at least, or less than 10, 20, 30, 40, 50, 60, 80, 100, 200 mM. The buffered solution can comprise about 15 mM Tris and 50 mM KCl. The nucleotides can comprise deoxyribonucleotide triphosphate molecules, including dATP, dCTP, dGTP, dTTP, in concentrations of about, more than, at least, or less than 5, 10, 15, 20, 25, 50, 100, 200, 300, 400, 500, 600, or 700 μM each. In some cases, a non-canonical nucleotide, e.g., dUTP is added to amplification reaction to a concentration of about, more than, at least, or less than 5, 10, 15, 20, 25, 50, 100, 200, 300, 400, 500, 600, or 700, 800, 900, or 1000 μM. In some cases, magnesium chloride (MgCl₂) is added to an amplification reaction at a concentration of about, more than, at least, or less than 1.0, 2.0, 3.0, 4.0, or 5.0 mM. The concentration of MgCl₂ can be about 3.2 mM.

A non-specific blocking agent such as BSA or gelatin from bovine skin can be used, wherein the gelatin or BSA is present in a concentration range of approximately 0.1 to about 0.9% w/v. Other possible blocking agents can include betalactoglobulin, casein, dry milk, or other common blocking agents. In some cases, preferred concentrations of BSA and gelatin are about 0.1% w/v.

An amplification reaction can also comprise one or more additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors). The one or more additives can include, e.g., 2-pyrrolidone, acetamide, N-methylpyrolidone (NMP), B-hydroxyethylpyrrolidone (HEP), propionamide, NN-dimethylacetamide (DMA), N-methylformamide (MMP), NN-dimethylformamide (DMF), formamide, N-methylacetamide (MMA), dimethyl sulfoxide (DMSO), polyethylene glycol, betaine, tetramethylammonium chloride (TMAC), 7-deaza-2′-deoxyguanosine, bovine serum albumin (BSA), T4 gene 32 protein, glycerol, or nonionic detergent (Triton X-100, Tween 20, Nonidet P-40 (NP-40), Tween 40, SDS (e.g., about 0.1% SDS)), salmon sperm DNA, sodium azide, betaine (N,N,N-trimethylglycine; [carboxymethyl]trimethylammonium), formamide, trehalose, dithiothreitol (DTT), betamercaptoethanol (BME), a plant polysaccharide, or an RNase inhibitor.

An amplification reaction can comprise one or more buffers. The one or more buffers can comprise, e.g., TAPS, bicine, Tris, Tricine, TAPSO, HEPES, TES, MOPS, PIPES, cacodylate, SSC, ADA, ACES, cholamine chloride, acetamidoglycine, glycinamide, maleate, phosphate, CABS, piperdine, glycine, citrate, glycylglycine, malate, formate, succinate, acetate, propionate, pyridine, piperazine, histidine, bis-tris, ethanolamine, carbonate, MOPSO, imidazole, BIS-TRIS propane, BES, MOBS, triethanolamine (TEA), HEPPSO, POPSO, hydrazine, Trizma (tris), EPPS, HEPPS, bicine, HEPBS, AMPSO, taurine (AES), borate, CHES, 2-amino-2-methyl-1-propanol (AMP), ammonium hydroxide, methylamine, or MES.

A non-ionic Ethylene Oxide/Propylene Oxide block copolymer can be added to an amplification reaction in a concentration of about 0.1%, 0.2%, 0.3%, 0.4%, 0.5%, 0.6%, 0.7%, 0.8%, 0.9%, or 1.0%. Common biosurfactants include non-ionic surfactants such as Pluronic F-68, Tetronics, Zonyl FSN. Pluronic F-68 can be present at a concentration of about 0.5% w/v.

In some cases magnesium sulfate can be substituted for magnesium chloride, at similar concentrations. A wide range of common, commercial PCR buffers from varied vendors can be substituted for the buffered solution.

Detection

Fluorescence detection can be achieved using a variety of detector devices equipped with a module to generate excitation light that can be absorbed by a fluorescer, as well as a module to detect light emitted by the fluorescer. In some cases, samples (such as droplets) can be detected in bulk. For example, samples can be allocated in plastic tubes that are placed in a detector that measures bulk fluorescence from plastic tubes. In some cases, one or more samples (such as droplets) can be partitioned into one or more wells of a plate, such as a 96-well or 384-well plate, and fluorescence of individual wells can be detected using a fluorescence plate reader.

In some cases, a detector further comprises handling capabilities for droplet samples, with individual droplets entering the detector, undergoing detection, and then exiting the detector. For example, a flow cytometry device can be adapted for use in detecting fluorescence from droplet samples. In some cases, a microfluidic device equipped with pumps to control droplet movement is used to detect fluorescence from droplets in single file. In some cases, droplets are arrayed on a two-dimensional surface and a detector moves relative to the surface, detecting fluorescence at each position containing a single droplet.

Computers

Following acquisition of fluorescence detection data, a computer can be used to store and process the data. A computer-executable logic can be employed to perform such functions as subtraction of background fluorescence, assignment of target and/or reference sequences, and quantification of the data. A computer can be useful for displaying, storing, retrieving, or calculating diagnostic results from the molecular profiling; displaying, storing, retrieving, or calculating raw data from genomic or nucleic acid expression analysis; or displaying, storing, retrieving, or calculating any sample or patient information useful in the methods described herein.

Also provided herein is software (computer readable medium) that comprises instructions that when executed on a computer can cause the computer to execute an algorithm that can analyze digital PCR data and next generation sequencing data to provide a map of a chromosome or region of a chromosome. A computer readable medium can comprise instructions recorded on the computer readable medium suitable for use in an electronic device, e.g., a computer, computer network server, portable electronic device, or electronic device described herein. The computer readable medium can be a non-transitory computer readable medium. Computer readable media can be configured to include data or computer executable instructions for manipulating data. The computer executable instructions can include data structures, objects, programs, routines, or other program modules that can be accessed by a processing system, such as one associated with a general purpose computer capable of performing different functions or one associated with a special purpose computer capable of performing a limited number of functions. Computer executable instructions can cause a processing system to perform a particular function or group of functions and are examples of program codes for implementing steps for methods disclosed herein. A particular sequence of executable instructions can provide an example of corresponding acts that can be used to implement such steps. Computer readable media includes, e.g., a hard disk, diskette, random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), CD±R, CD±RW, DVD, DVD±RW, DVD±R, DVD-RAM, HD DVD, HD DVDR, HD DVD±RW, HD DVD±RAM, Blu-ray Disc, optical or magnetic storage medium, paper tape, punch cards, optical mark sheets or any other device that is capable of providing data or executable instructions that can be accessed by a processing system. Computer readable medium are described, e.g., in U.S. Pat. No. 7,783,072.

Computer code devices can include, e.g., scripts, dynamic link libraries (DLLs), interpretable programs, Java classes and applets, Common Object Request Broker Architecture (COBRA), or complete executable programs.

In some cases, chromosome mapping comprises use of a computer implemented algorithm. In some cases, the mapping comprises inputting linkage frequencies and next generation sequencing data into a computer implemented algorithm.

Also provided herein are systems for mapping chromosomes. The system can comprise instrumentation for extracting nucleic acid from a sample, sequencing nucleic acid (e.g., next generation sequencing); amplifying nucleic acid (e.g., digital PCR, droplet digital PCR), analyzing sequencing and/or amplification data, and/or instrumentation for mapping a chromosome. Systems provided herein can comprise one or more electronic devices that are in electronic communication. The one or more electronic devices can be connected by a wireless and/or wired connection.

A report can be generated using the methods, compositions, and kits described herein. For example, a report can comprise chromosome mapping information. The map can comprise information on distances between loci and degree of amplification of loci. This information can be useful for understanding disease (e.g., autoimmune disease, neurodegenerative disease, cancer) and health traits, as well as responses of an organism to the environment (e.g., exposure to toxins, viruses (e.g., smallpox, influenza), treatment with a drug (e.g., an anesthetic, antibiotic, antidepressant, antidiabetic agent, antiemetic, antihistamine, anti-infective agent, antineoplastic, antiparkisonian drug, antirheumatic agent, antipsychotic, anxiolytic, cardiovascular agent, central nervous system stimulant, drug for Alzheimer's disease management, a cold medication, COPD (chronic obstructive pulmonary disease) drug, dietary supplement, drug for erectile dysfunction, gastrointestinal agent, hormone, drug for the treatment of alcoholism, immunosuppressive agent, migraine preparation, muscle relaxant, drug for treating myocardial infarction, nonsteroidal anti-inflammatory agent, opioid, other analgesic and stimulant, opthalmic preparation, osteoporosis preparation, pain medication, panic medication, prostaglandin, respiratory agent, sedative, skin and mucous membrane agent, insomnia medication, weight loss drug, and vertigo agent; response to an attack with a bioterrorist agent (e.g., anthrax, smallpox, influenza), or stress.

Digital Analysis

A digital readout assay, e.g., digital PCR, can be used to count targets (e.g., target nucleic acid sequences) by partitioning the targets in a sample and identifying partitions containing the target. A digital readout is an all or nothing analysis in that it specifies whether a given partition contains the target of interest, but does not necessarily indicate how many copies of the target are in the partition. For example, a single polynucleotide containing two targets can be in a partition, but under normal analysis conditions, the partition will only be considered to contain one target. If the targets on the same polynucleotide are separated by a large number of base pairs, some of the target nucleic acid sequences may be separated by fragmentation during purification of a sample—some linked target nucleic acid sequences may not remain physically linked after sample preparation. Digital PCR is described generally, e.g., at Vogelstein and Kinzler (1999) PNAS 96:9236-9241. Applications of this technology include, e.g., high-resolution CNV measurements, follow-up to genome-wide association studies, cytogenetic analysis, CNV alterations in cancerous tissue, and CNV linkage analysis.

In general, dPCR can involve spatially isolating (or partitioning) individual polynucleotides from a sample and carrying out a polymerase chain reaction on each partition. The partition can be, e.g., a well (e.g., wells of a microwell plate), capillary, dispersed phase of an emulsion, a chamber (e.g., a chamber in an array of miniaturized chambers), a droplet, or a nucleic acid binding surface. The sample can be distributed so that each partition has about 0, 1, or 2 target polynucleotides. Each partition can have, on average, less than 5, 4, 3, 2, or 1 copies of a target nucleic acid per partition (e.g., droplet). In some cases, at least 0, 1, 2, 3, 4, 5, 6, 7, 8, 9, 10, 20, 30, 40, 50, 60, 70, 80, 90, 100, 125, 150, 175, or 200 partitions (e.g., droplets) have zero copies of a target nucleic acid. After PCR amplification, the number of partitions with or without a PCR product can be enumerated. The total number of partitions can be about, less than, at least, or more than, 500, 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 150,000, 200,000, 500,000, 750,000, or 1,000,000. The total number of partitions can be about 500 to about 1,000,000, about 500 to about 500,000, about 500 to about 250,000, about 500 to about 100,000, about 1000 to about 1,000,000, about 1000 to about 500,000, about 1000 to about 250,000, about 1000 to about 100,000, about 10,000 to about 1,000,000, about 10,000 to about 100,000, or about 10,000 to about 50,000.

In some cases, the digital PCR is droplet digital PCR. In some embodiments of a droplet digital PCR experiment, less than 0.00001, 0.00005, 0.00010, 0.00050, 0.001, 0.005, 0.01, 0.05, 0.1, 0.5, 1, 2, 2.5, 3, 3.5, 4, 4.5, 5, 6, 7, 8, 9, or 10 copies of target polynucleotide can detected. In some cases, less than 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 150, 200, 250, 300, 350, 400, 450, or 500 copies of a target polynucleotide are detected. In some cases, the droplets described herein are generated at a rate of greater than 1, 2, 3, 4, 5, 10, 50, 100, 200, 300, 400, 500, 600, 700, 800, 900, or 1000 droplets/second.

Droplet digital PCR (ddPCR) can offer a practical solution for validating copy number variations identified by next generation sequencers and microarrays. Methods using ddPCR™ can empower one person to screen many samples, e.g., hundreds of samples, for CNV analysis in a single work shift. In one embodiment, a ddPCR workflow is provided that involves using one or more restriction enzymes to separate tandem copies of a target nucleic acid sequence prior to assembling a duplex TaqMan® assay that includes reagents to detect both the target nucleic acid sequence (e.g., a first gene) and a single-copy reference nucleic acid sequence (e.g., a second gene). When ddPCR is used, the reaction mixture can then be partitioned into about, at least, less than, or more than, 500, 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 150,000, 200,000, 500,000, 750,000, 1,000,000, 2,000,000, 3,000,000, 4,000,000, 5,000,000, 6,000,000, 7,000,000, 8,000,000, 9,000,000, or 10,000,000 nanoliter droplets that can be thermo-cycled to end-point before being analyzed. In some cases, the droplets are greater than one nanoliter, in other cases, the droplets are less than one nanoliter (e.g., picoliter). The number of droplets per reaction can be about 1000 to about 1,000,000, about 1000 to about 750,000, about 1000 to about 500,000, about 1000 to about 250,000, about 1000 to about 100,000, about 1000 to about 50,000, about 1000 to about 30,000, about 1000 to about 10,000, about 10,000 to about 1,000,000, about 10,000 to about 750,000, about 10,000 to about 500,000, about 10,000 to about 250,000, about 10,000 to about 100,000, about 10,000 to about 50,000, or about 10,000 to about 30,000. The number of droplets per reaction can be about 20,000 to about 1,000,000, about 20,000 to about 750,000, about 20,000 to about 500,000, about 20,000 to about 250,000, about 20,000 to about 200,000, about 20,000 to about 50,000, about 50,000 to about 100,000, about 50,000 to about 200,000; or about 50,000 to about 300,000.

An analysis can occur in a two-color reader. The fraction of positive-counted droplets can enable the absolute concentrations for the target and reference nucleic acid sequences (e.g., genes) to be measured. This information can be used to determine a relative copy number. For example, at least 20,000 PCR replicates per well can provide the statistical power to resolve higher-order copy number differences. This low-cost method can reliably generate copy number measurements with 95% confidence intervals that span integer without overlap of adjacent copy number states. This technology is capable of determining the linkage of copy number variants, and it can be used to determine whether gene copies are on the same or different chromosomes.

The volumes may have any suitable size. In some cases, the volumes can have a diameter or characteristic cross-sectional dimension of about 10 to 1000 micrometers.

Nucleic acid that is partitioned can have any suitable characteristics. The nucleic acid may include genetic material of the subject (e.g., the subject's genomic DNA and/or RNA), messenger RNA of the subject, and/or cDNA derived from RNA of the subject, among others. The nucleic acid may have any suitable average length. Generally, the average length is substantially greater than the distance on a chromosome between the polymorphic loci to be analyzed. With this average length, alleles linked in the subject are also linked frequently in the isolated nucleic acid and thus tend to distribute together to the same volumes when the aqueous phase is partitioned. In some cases, each primer set can be capable of amplifying at least a pair of distinct alleles from a polymorphic locus.

Each volume can be partitioned to contain any suitable average concentration of nucleic acid. Generally, the process of partitioning, in combination with a suitable starting concentration of the nucleic acid in the aqueous phase, produces volumes that have an average of less than several genome equivalents of the nucleic acid per volume. Although the method can be performed with an average of more than one genome equivalent per volume (e.g., about two genome equivalents per volume), the analysis generally becomes more efficient and reliable, with less background, by limiting the concentration to an average of less than one genome equivalent per volume. Accordingly, each volume can contain on average less than one copy or molecule of a target region that includes each polymorphic locus and/or an average of less than one copy of any allele sequence of each polymorphic locus.

An integrated, rapid, flow-through thermal cycler device can be used in the methods described herein. See. e.g., International Application No. PCT/US2009/005317, filed Sep. 23, 2009. In such an integrated device, a capillary is wound around a cylinder that maintains 2, 3, or 4 temperature zones. As droplets flow through the capillary, they are subjected to different temperature zones to achieve thermal cycling. The small volume of each droplet results in an extremely fast temperature transition as the droplet enters each temperature zone.

A digital PCR device (e.g., droplet digital PCR device) for use with the methods, compositions, and kits described herein can detect multiple signals (see e.g. U.S. Provisional Patent Application No. 61/454,373, filed Mar. 18, 2011, herein incorporated by reference in its entirety).

Droplet digital PCR can involve the generation of thousands of discrete, robust microdroplet reactors per second. ddPCR can involve standard thermal cycling with installed-base instruments, which can make digital data accessible immediately to researchers. Rapid interrogation of each droplet can yield counts of target molecules present in the initial sample.

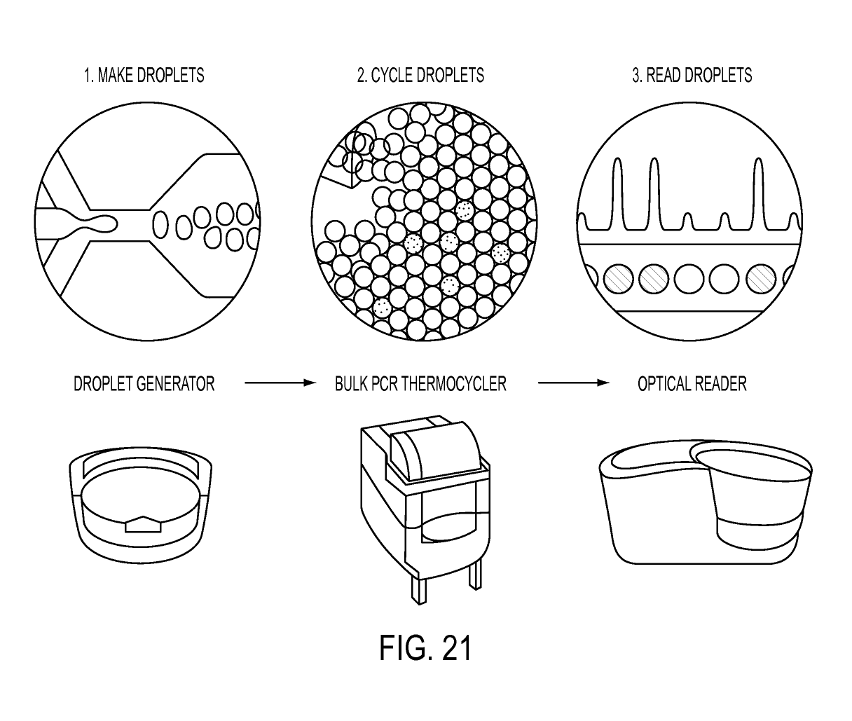

FIG. 21 illustrates an example of a general workflow for a ddPCR experiment. As shown in FIG. 21, the process can start by partitioning a sample into multiple partitions (e.g., droplets), followed by thermal cycling the sample in a thermal cycler. The fluorescence of the droplets can then be detected using a reader (e.g., an optical reader).

Droplet Generation

The present disclosure includes compositions and methods using droplet digital PCR The droplets described herein include emulsion compositions (or mixtures of two or more immiscible fluids) described in U.S. Pat. No. 7,622,280, and droplets generated by devices described in International Application No. PCT/US2009/005317, filed Sep. 23, 2009. The term emulsion, as used herein, can refer to a mixture of immiscible liquids (such as oil and water). Oil-phase and/or water-in-oil emulsions allow for the compartmentalization of reaction mixtures within aqueous droplets. The emulsions can comprise aqueous droplets within a continuous oil phase. The emulsions provided herein can be oil-in-water emulsions, wherein the droplets are oil droplets within a continuous aqueous phase. The droplets provided herein are designed to prevent mixing between compartments, with each compartment protecting its contents from evaporation and coalescing with the contents of other compartments.

The mixtures or emulsions described herein can be stable or unstable. The emulsions can be relatively stable and have minimal coalescence. Coalescence occurs when small droplets combine to form progressively larger ones. In some cases, less than 0.00001%, 0.00005%, 0.00010%, 0.00050%, 0.001%, 0.005%, 0.01%, 0.05%, 0.1%, 0.5%, 1%, 2%, 2.5%, 3%, 3.5%, 4%, 4.5%, 5%, 6%, 7%, 8%, 9%, or 10% of droplets generated from a droplet generator coalesce with other droplets. The emulsions can also have limited flocculation, a process by which the dispersed phase comes out of suspension in flakes.

Splitting a sample into small reaction volumes as described herein can enable the use of reduced amounts of reagents, thereby lowering the material cost of the analysis. Reducing sample complexity by partitioning also improves the dynamic range of detection because higher-abundance molecules are separated from low-abundance molecules in different compartments, thereby allowing lower-abundance molecules greater proportional access to reaction reagents, which in turn enhances the detection of lower-abundance molecules.

Droplets can be generated having an average diameter of about, at least, less than, or more than 0.001, 0.01, 0.05, 0.1, 1, 5, 10, 20, 30, 40, 50, 60, 70, 80, 100, 120, 130, 140, 150, 160, 180, 200, 300, 400, or 500 microns. Droplets can have an average diameter of about 0.001 to about 500, about 0.01 to about 500, about 0.1 to about 500, about 0.1 to about 100, about 0.01 to about 100, or about 1 to about 100 microns. Microfluidic methods of producing emulsion droplets using microchannel cross-flow focusing or physical agitation are known to produce either monodisperse or polydisperse emulsions. The droplets can be monodisperse droplets. The droplets can be generated such that the size of said droplets does not vary by more than plus or minus 5% of the average size of said droplets. In some cases, the droplets are generated such that the size of said droplets does not vary by more than plus or minus 2% of the average size of said droplets. A droplet generator can generate a population of droplets from a single sample, wherein none of the droplets vary in size by more than plus or minus about 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the average size of the total population of droplets.

Higher mechanical stability can be useful for microfluidic manipulations and higher-shear fluidic processing (e.g., in microfluidic capillaries or through 90 degree turns, such as valves, in a fluidic path). Pre- and post-thermally treated droplets or capsules can be mechanically stable to standard pipet manipulations and centrifugation.

A droplet can be formed by flowing an oil phase through an aqueous sample. The aqueous phase can comprise a buffered solution and reagents for performing a PCR reaction, including nucleotides, primers, probe(s) for fluorescent detection, template nucleic acids, DNA polymerase enzyme, and optionally, reverse transcriptase enzyme.

The aqueous phase can comprise one or more buffers and/or additives described herein.

Primers for amplification within the aqueous phase can have a concentration of about, at least, more than, or less than 0.05, 0.1, 0.2, 0.3, 0.4, 0.5, 0.6, 0.7, 0.8, 0.9, 1.0, 1.2, 1.5, 1.7, or 2.0 μM. Primer concentration within the aqueous phase can be about 0.05 to about 2, about 0.1 to about 1.0, about 0.2 to about 1.0, about 0.3 to about 1.0, about 0.4 to about 1.0, or about 0.5 to about 1.0 μM. The concentration of primers can be about 0.5 μM. The aqueous phase can comprise one or more probes for fluorescent detection, at a concentration of about, at least, more than, or less than 0.05, 0.1, 0.2, 0.3, 0.4, 0.5, 0.6, 0.7, 0.8, 0.9, 1.0, 1.2, 1.4, 1.6, 1.8, or 2.0 μM. The aqueous phase can comprise one or more probes for fluorescent detection, at a concentration of about 0.05 to about 2.0, about 0.1 to about 2.0, about 0.25 to about 2.0, about 0.5 to about 2.0, about 0.05 to about 1, about 0.1 to about 1, or about 0.1 to about 0.5 μM. The concentration of probes for fluorescent detection can be about 0.25 μM. Amenable ranges for target nucleic acid concentrations in PCR can be between about 1 pg and about 500 ng.

The oil phase can comprise a fluorinated base oil which can be additionally stabilized by combination with a fluorinated surfactant such as a perfluorinated polyether. In some cases, the base oil can be one or more of HFE 7500, FC-40, FC-43, FC-70, or another common fluorinated oil. In some cases, the anionic surfactant is Ammonium Krytox (Krytox-AM), the ammonium salt of Krytox FSH, or morpholino derivative of Krytox-FSH. Krytox-AS can be present at a concentration of about, more than, at least, or less than 0.1%, 0.2%, 0.3%, 0.4%, 0.5%, 0.6%, 0.7%, 0.8%, 0.9%, 1.0%, 2.0%, 3.0%, or 4.0% w/w. In some cases, the concentration of Krytox-AS is 1.8%. In other cases, the concentration of Krytox-AS is 1.62%. Morpholino derivative of Krytox-FSH can be present at a concentration of about 0.1%, 0.2%, 0.3%, 0.4%, 0.5%, 0.6%, 0.7%, 0.8%, 0.9%, 1.0%, 2.0%, 3.0%, or 4.0% w/w. The concentration of morpholino derivative of Krytox-FSH can be about 1.8%. The concentration of morpholino derivative of Krytox-FSH can be about 1.62%.

The oil phase can further comprise an additive for tuning the oil properties, such as vapor pressure or viscosity or surface tension. Nonlimiting examples include perfluoro-octanol and 1H,1H,2H,2H-Perfluorodecanol. 1H, 1H,2H,2H-Perfluorodecanol can be added to a concentration of about, more than, at least, or less than 0.05%, 0.06%, 0.07%, 0.08%, 0.09%, 1.00%, 1.25%, 1.50%, 1.75%, 2.00%, 2.25%, 2.50%, 2.75%, or 3.00% w/w. 1H,1H,2H,2H-Perfluorodecanol can be added to a concentration of about 0.18% w/w.

The emulsion can be formulated to produce highly monodisperse droplets having a liquid-like interfacial film that can be converted by heating into microcapsules having a solid-like interfacial film; such microcapsules can behave as bioreactors able to retain their contents through a reaction process such as PCR amplification. The conversion to microcapsule form can occur upon heating. For example, such conversion can occur at a temperature of greater than about 50, 60, 70, 80, 90, or 95° C. In some cases this heating occurs using a thermocycler. During the heating process, a fluid or mineral oil overlay can be used to prevent evaporation. Excess continuous phase oil may or may not be removed prior to heating. The biocompatible capsules can be resistant to coalescence and/or flocculation across a wide range of thermal and mechanical processing.

Following conversion, the capsules can be stored at about, more than, at least, or less than 3, 4, 5, 6, 7, 8, 9, 10, 15, 20, 25, 30, 35, or 40° C. These capsules can be useful in biomedical applications, such as stable, digitized encapsulation of macromolecules, particularly aqueous biological fluids containing a mix of nucleic acids or protein, or both together; drug and vaccine delivery; biomolecular libraries; clinical imaging applications, and others.

The microcapsules can contain one or more polynucleotides and may resist coalescence, particularly at high temperatures. Accordingly, PCR amplification reactions can occur at a very high density (e.g., number of reactions per unit volume). In some cases, greater than about 100,000, 500,000, 1,000,000, 1,500,000, 2,000,000, 2,500,000, 5,000,000, or 10,000,000 separate reactions can occur per ml. In some cases, the reactions occur in a single well, e.g., a well of a microtiter plate, without inter-mixing between reaction volumes. The microcapsules can also contain other components to enable a PCR reaction to occur, e.g., primers, probes, dNTPs, DNA or RNA polymerases, etc. These capsules exhibit resistance to coalescence and flocculation across a wide range of thermal and mechanical processing.

In one embodiment, droplet generation can be improved after the size of DNA is reduced by, e.g., digestion, heat treatment, or shearing.

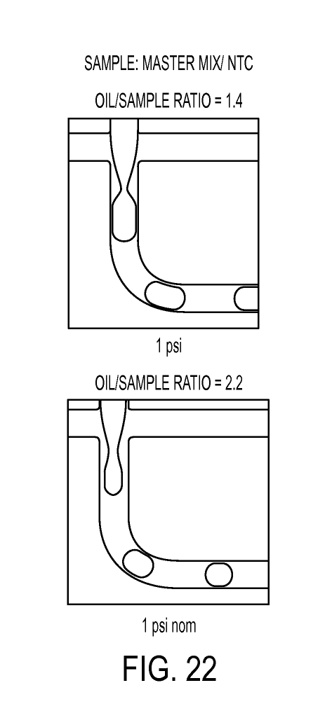

FIG. 22 displays several images of droplets showing a) droplet formation as a droplet is pinched by inflow of oil from the sides and b) stretching/necking down as the droplet pulls away from the bulk fluid

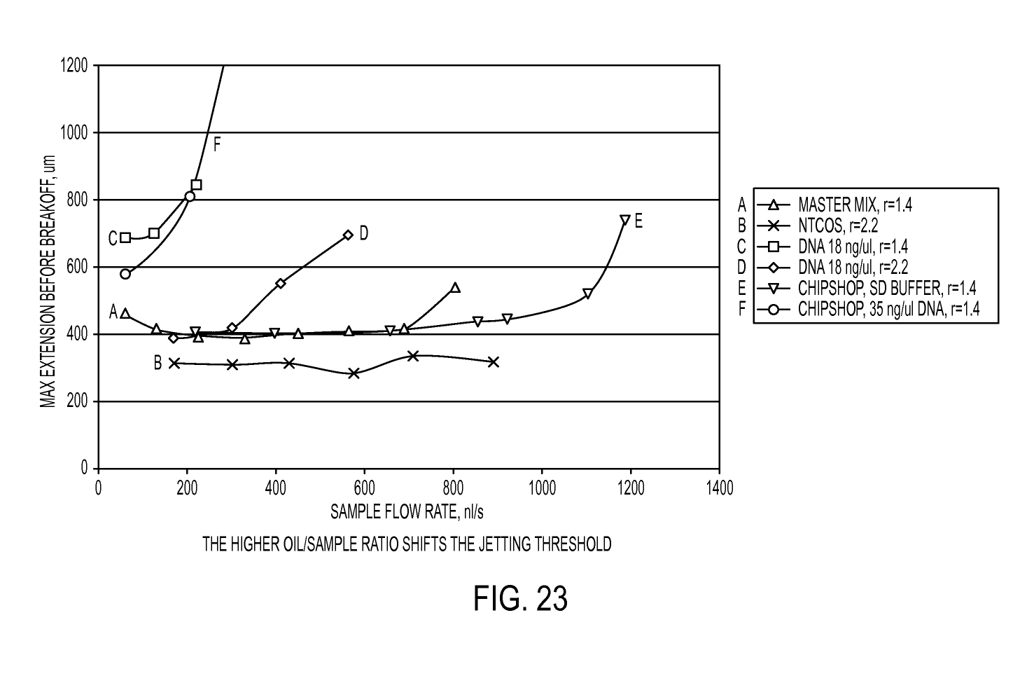

FIG. 23 shows the effect of increasing DNA load. FIG. 23 plots maximum extension versus flow rate. Extension is measured from the center of the cross to the farthest extent of the droplet just as it breaks off. Some droplet extension is tolerable, but if it becomes excessive, a long “thread” is drawn that connects the droplet to the bulk fluid. As the droplet breaks off, this thread may collapse to microdroplets, leading to undesirable polydispersity. In extreme cases, the droplet does not break off; instead the aqueous phase flows as a continuous phase down the center of the channel, while the oil flows along the channel walls, and no droplets are formed.

One way to decrease extension is to decrease flow rate. Decreasing flow rate can have the undesirable side effects of lower throughput and also increased droplet size. The purple (B), teal (E) and green (A) curves have zero DNA. These samples can tolerate high flow rates without substantially increasing their extension into the channel.

The blue (D), orange (F) and red (C) curves have higher DNA loads. For these conditions, higher flow rates cause droplet extension into the channel. Low flow rates can be used to avoid excessive droplet extension.

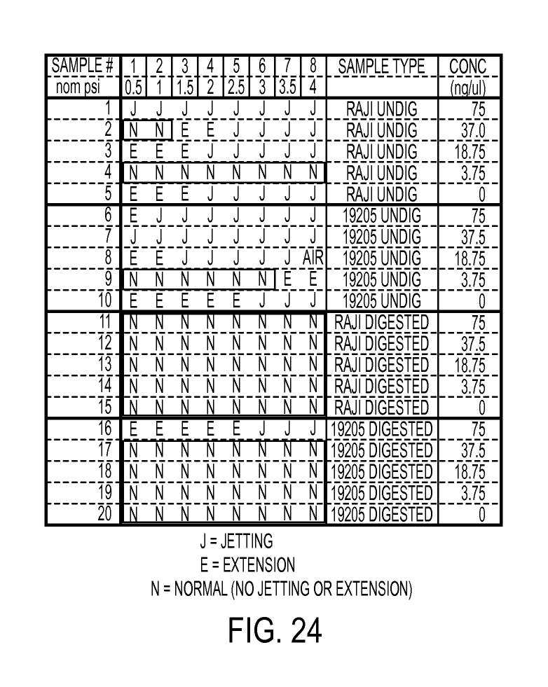

FIG. 24 shows undigested samples 1-10 and digested samples 11-20 in an experiment to investigate droplet properties. DNA load is shown in the right-most column; pressure (roughly proportional to flow rate) is shown in the 2nd row. The table is color and letter coded: J (RED) indicates jetting, E (YELLOW) indicates extension, and N (GREEN) indicates normal (no jetting or extension) droplet generation. As can be seen, digestion (with restriction enzymes) resulted in improved droplet generation, even at high DNA loads and high flow rates.

Applications

The methods described herein can be used for diagnosing or prognosing a disorder or disease.

The methods and compositions provided herein can be useful for both human and non-human subjects. The applications of the methods and compositions provided herein are numerous, e.g., high-resolution CNV measurements, follow-up to genome-wide association studies, cytogenetic analysis, CNV alterations in cancerous tissue, CNV linkage analysis, as well as haplotype analysis.

The applications provided herein include applications for diagnosing, predicting, determining or assessing the genetic characteristics of a fetus or embryo. In some cases, the applications can be used to diagnose, predict, determine, or assess the nucleic acids in an embryo produced by in vitro fertilization or other assisted reproductive technology. Furthermore, the methods provided herein can be used to provide information to an expectant parent (e.g., a pregnant woman) in order to assess CNV or genetic phasing within the genome of a developing fetus. In other cases, the methods provided herein can be used to help counsel patients as to possible genetic attributes of future offspring. In some cases, the methods can be used in connection with an Assisted Reproductive Technology. For example, the information can be used to assess CNV or genetic phasing in a sample taken from an embryo produced by in vitro fertilization.

One or more CNVs can be found in a cancer cell. For example, EGFR copy number can be increased in non-small cell lung cancer. CNVs can be associated with efficacy of a therapy. For example, increased HER2 gene copy number can enhance the response to gefitinib therapy in advanced non-small cell lung cancer. See Cappuzzo F. et al. (2005) J. Clin. Oncol. 23: 5007-5018. High EGFR gene copy number can predict for increased sensitivity to lapatinib and capecitabine. See Fabi et al. (2010) J. Clin. Oncol. 28:15s (2010 ASCO Annual Meeting). High EGFR gene copy number is associated with increased sensitivity to cetuximab and panitumumab.

In one embodiment, a method is provided comprising determining number of copies of a target sequence using a method described herein, and designing a therapy based on said determination. In one embodiment, the target is EGFR, and the therapy comprises administration of cetuximab, panitumumab, lapatinib, and/or capecitabine. In another embodiment, the target is ERBB2, and the therapy comprises trastuzumab (Herceptin).

Copy number variation can contribute to genetic variation among humans. See e.g. Shebat J. et al. (2004) Science 305: 525-528.

Diseases associated with copy number variations can include, for example, DiGeorge/velocardiofacial syndrome (22q11.2 deletion), Prader-Willi syndrome (15q11-q13 deletion), Williams-Beuren syndrome (7q11.23 deletion), Miller-Dieker syndrome (MDLS) (17p13.3 microdeletion), Smith-Magenis syndrome (SMS) (17p11.2 microdeletion), Neurofibromatosis Type 1 (NF1) (17q11.2 microdeletion), Phelan-McErmid Syndrome (22q113 deletion), Rett syndrome (loss-of-function mutations in MECp2 on chromosome Xq28), Merzbacher disease (CNV of PLP1), spinal muscular atrophy (SMA) (homozygous absence of telomerec SMN1 on chromosome 5q13), Potocki-Lupski Syndrome (PTLS, duplication of chromosome 17p.11.2). Additional copies of the PMP22 gene can be associated with Charcot-Marie-Tooth neuropathy type IA (CMT1A) and hereditary neuropathy with liability to pressure palsies (HNPP). The methods of detecting CNVs described herein can be used to diagnose CNV disorders described herein and in publications incorporated by reference. The disease can be a disease described in Lupski J. (2007) Nature Genetics 39: S43-S47.

Aneuploides, e.g., fetal aneuploidies, can include, e.g., trisomy 13, trisomy 18, trisomy 21 (Down Syndrome), Klinefelter Syndrome (XXY), monosomy of one or more chromosomes (X chromosome monosomy, Turner's syndrome), trisomy X, trisomy of one or more chromosomes, tetrasomy or pentasomy of one or more chromosomes (e.g., XXXX, XXYY, XXXY, XYYY, XXXXX, XXXXY, XXXYY, XYYYY and XXYYY), triploidy (three of every chromosome, e.g. 69 chromosomes in humans), tetraploidy (four of every chromosome, e.g. 92 chromosomes in humans), and multiploidy. In some embodiments, an aneuploidy can be a segmental aneuploidy. Segmental aneuploidies can include, e.g., 1p36 duplication, dup(17)(p11.2p11.2) syndrome, Down syndrome, Pelizaeus-Merzbacher disease, dup(22)(q11.2q11.2) syndrome, and cat-eye syndrome. In some cases, an abnormal genotype, e.g., fetal genotype, is due to one or more deletions of sex or autosomal chromosomes, which can result in a condition such as Cri-du-chat syndrome, Wolf-Hirschhorn, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, Steroid sulfatase deficiency, Kallmann syndrome, Microphthalmia with linear skin defects, Adrenal hypoplasia, Glycerol kinase deficiency, Pelizaeus-Merzbacher disease, Testis-determining factor on Y, Azospermia (factor a), Azospermia (factor b), Azospermia (factor c), or 1p36 deletion. In some embodiments, a decrease in chromosomal number results in an XO syndrome.

Excessive genomic DNA copy number variation was found in Li-Fraumeni cancer predisposition syndrome (Shlien et al. (2008) PNAS 105:11264-9). CNV is associated with malformation syndromes, including CHARGE (coloboma, heart anomaly, choanal atresia, retardation, gential, and ear anomalies), Peters-Plus, Pitt-Hopkins, and thrombocytopenia-absent radius syndrome (see e.g., Ropers H H (2007) Am J of Hum Genetics 81: 199-207). The relationship between copy number variations and cancer is described, e.g., in Shlien A. and Malkin D. (2009) Genome Med. 1(6): 62. Copy number variations are associated with, e.g., autism, schizophrenia, and idiopathic learning disability. See e.g., Sebat J., et al. (2007) Science 316: 445-9; Pinto J. et al. (2010) Nature 466: 368-72; Cook E. H. and Scherer S. W. (2008) Nature 455: 919-923; Ruderfer D. et al. (2013) European Journal of Human Genetics doi:10.1038/ejhg.2012.287.

Copy number variations can be associated with resistance of cancer patients to certain therapeutics. For example, amplification of thymidylate synthase can result in resistance to 5-fluorouracil treatment in metastatic colorectal cancer patients. See Wang et al. (2002) PNAS USA vol. 99, pp. 16156-61.

High copy number of CCL3L1 is associated with lower susceptibility to HIV infection (Gonzalez E. et al. (2005) Science 307: 1434-1440). Low copy number of FCGR3B (CD16 cell surface immunoglobulin receptor) can increase susceptibility to systemic lupus erythematosus (Aitman T. J. et al. (2006) Nature 439: 851-855). Autosomal-dominant microtia was found to be linked to five tandem copies of a copy-number-variable region at chromosome 4p16 (Balikova I. (2008) Am J. Hum Genet. 82: 181-187). The methods, compositions, and kits described herein can be used to investigate any of these conditions.

Individuals from populations with high-starch diets generally have more amylase gene (AMY1) copies than individuals from populations with low-starch diets (Perry H. et al. (2007) Nature Genetics 39:1256-1260). Thus, copy number can be subject to positive selection during evolution. The methods, compositions, and kits described herein can be used to study evolution.

Other examples of copy number variations associated with disease include, e.g., trisomy 21 (Down Syndrome), trisomy 18 (Edwards syndrome), and trisomy 13 (Patau syndrome).

Determining whether nucleic acids are linked or separated (fragmented) can provide useful information for a variety of applications. For example, the methods described herein can be used to diagnose or prognose a disorder or disease, for example, a genetic disorder. The methods described herein can be used to diagnose and prognose fetal disorders, e.g., fetal aneuploidy.

The methods described herein can be useful for evaluating an infection, e.g., a viral or bacterial infection. For example, the methods can be used to determine whether two or more mutations lie within a single virus or bacterium or whether two or more mutations are in different individual viruses or bacteria.

The methods described herein can be useful monitoring the generation of a transgenic animal. For example, the methods can be used to determine whether a transgene has been introduced once or multiple times into the genome of a transgenic organism. In other embodiments, the methods can be useful monitoring the generation of a knockout animal. For example, the methods can be used to determine whether a gene has been deleted or interrupted in a knockout organism. The knockout animals can be whole-body knockout animals (e.g., the gene is deleted or interrupted in all tissues), tissue-specific knockout animals (e.g. the gene is deleted or interrupted in specific tissues), or inducible knockout animals (e.g., deletion or interruption of the gene can be induced by reagents). In some cases, the methods can be useful monitoring the generation of knock-in animals. For example, the methods can be used to determine whether a transgene has been introduced once or multiple times into the genome of a knockout animal.

Checkpoints, DNA Damage, and the Cell Cycle

Determining whether loci are linked or separated (fragmented) can be used to study DNA damage repair, double strand break repair, homologous recombination, microhomology-mediated end joining, single-strand annealing (SSA), breakage-induced replication, or non-homologous end joining (NHEJ). The methods described herein can be used to diagnose and prognose diseases associated with these processes.

DNA damage can arise from environmental factors and endogenous or normal metabolic processes. Endogenous factors that can damage DNA include, e.g., reactive oxygen species and replication errors. Physiologic double-strand DNA breaks can include V(D)J recombination breaks and class switch breaks. Pathologic double-strand DNA breaks can result from ionizing radiation, oxidative free radicals, replication across a nick, inadvertent enzyme action at fragile sites, topoisomerase failure, and mechanical stress. Environmental or exogenous factors that can cause DNA damage include ultraviolet radiation, x-rays, gamma-rays, DNA intercalating agents, some plant toxins, viruses, thermal disruption, and chemotherapy. Meiotic cells can have additional sources of DSBs, including the enzyme Spo11.

Double strand DNA breaks can be repaired by, e.g., NHEJ. Factors that can be involved in NHEJ include, e.g., Ku70/86, DNA-PKcs, Artemis, pol μ and λ XRCC4. DNA ligase IV, XRC44, and XLF-Cernunnos. After formation of a double-strand break, Ku can bind to the break to form a DNA complex. The DNA end complex can recruit nuclease, polymerase, and ligase activities. Ku at the end of DNA can form a stable complex with DNA-PKcs. DNA-PKcs can comprise 5′ endonuclease activity, 3′ endonuclease activity, and a hairpin opening activity. Artemis can comprise a 5′ endonuclease activity. The 3′ endonuclease of PALF (APLF) can play a role in NHEJ. Polymerase mu and lambda can bind Ku:DNA complexes through their BRCT domains. DNA ligase IV can ligate across gaps, ligate incompatible DNA ends, and ligate single-stranded DNA. NHEJ can involve strand resection. XRCC4 can tetramerize, and PNK (polynucleotide kinase), APTX (aprataxin, a protein that can play a role in deadenylation of aborted ligation products), and PALF can interact with XRCC4. Double-strand DNA break repair by NHEJ is review, e.g., in Lieber, M (2011) Annu. Rev. Biochem. 79: 181-211, which is hereby incorporated by reference in its entirety. NHEJ can occur at any time in the cell cycle.

NHEJ proteins can play a role in V(D)J recombination. The proteins RAG1 and RAG2 can play a role in V(D)J recombination. Class switch recombination can occur in B cells after completion of V(D)J recombination and can be used to change immunoglobulin heavy chain genes. This process can involve activation-induced deaminase (AID), RNase H, uracil glycosylase, APE1, and Exo1.

Double strand DNA breaks can be repaired by homology-directed repair (e.g., homologous recombination or single-strand annealing). Examples of factors that can be involved in these process include RAD50, MRE11, Nbs1 (collectively, the MRN complex); RAD51 (B, C, D), XRCC2, XRCC3, RAD52, RAD54B, and BRCA2. During the S and G2 phases of the cell cycle, there are two sister chromatids in close proximity, so homology-directed repair can be more common in these phases.

The ATM and ATR kinases can recognize damaged DNA. These kinases, along with DNA-PK, can phosphorylate H2AX and generate γH2AX foci. ATR can be activated by single-stranded DNA regions that result from replication fork stalling or the processing of bulky lesions. ATR can interact with ATRIP. The 9-1-1 complex (Rad9, Hus1, and Rad1) can play a role in substrate phosphorylation by ATR. RPA can bind ssDNA and can play a role in substrate phosphorylation by ATR.

ATM can recognize DNA ends through MRN. Phosphorylated H2AX can recruit MDC1, the ubiquitin ligases RNF8 and RNF168, and 53BP1. ATM can phosphorylate Chk2 and p53.

Checkpoints and cell cycle regulation can also be analyzed using the methods, compositions, and kits described herein. Cells can proceed through a cell cycle, and the cell cycle can comprise G1 phase, S phase (DNA synthesis), G2 phase, and M phase (mitosis). Cells that have stopped dividing can be in G0 phase (quiescence). Checkpoints can be used to halt the cell cycle and permit repair of DNA damage before the cell cycle is permitted to continue. A DNA damage checkpoint can occur at the boundaries of G1 and S phases and G2 and M phases. Another checkpoint is the intra-S phase checkpoint.

Other Processes

Determining whether nucleic acids are linked or separated (fragmented) can be used to study a polymerase (e.g., DNA polymerase, RNA polymerase, reverse transcriptase) in processes such as DNA replication and transcription. For example, the processivity of a polymerase can be determined (e.g., to determine the percentage of nascent strands that are full length versus partial length, one can measure how many truncated versions of a gene are present by counting the number of first half copies versus last half copies of a gene). Because synthesis occurs 5″ to 3″, it is expected that more of the 1st half (5′ end) of a product to be synthesized would be produced than the last half (3′ end).

Determining whether loci are linked or separated (fragmented) in a sample can be useful for studying one or more restriction enzymes, RNAzymes, DNAzymes, exonucleases, endonucleases, RNases, DNase, etc., to determine the efficiency of cleavage (e.g., separation to two linked targets) by these enzymes.

Determining whether genetic loci are linked or separated (fragmented) can be useful for studying RNA splicing, genetic rearrangement, localization of genes, and DNA rearrangement in cancer. The genetic rearrangement can be, e.g., a chromosomal translocation. The translocation can be a reciprocal (non-Robertsonian translocation), which can involve the exchange of material between nonhomologous chromosomes. The translocation can be a Roberstonian translocation. A Robertsonian translocation can involve a rearrangement of two acrocentric chromosomes that fuse near a centromere. Translocations associated with disease include, e.g. t(8:14)(q24: a32) (Burkitt's lymphoma; fusion of c-myc with IGH); t(11;14)(q13;q32) (Mantle cell lymphoma; fusion of cyclin D1 with IGH); t(14;18)(q32;q21) (follicular lymphoma; fusion of IGH with Bcl-2); t(10;(various)(q11;(various)) (papillary thyroid cancer; involves RET proto-oncogene on chromosome 10); t(2;3)(q13;p25) (follicular thyroid cancer, fusion of PAX8 with PPARγ1)): t(8:21)(q22:q22) (acute myeloblastic leukemia): t(9:22)(q34:q11) Philadelphia chromosome (chronic myelogenous leukemia; acute lymphoblastic leukemia; fusion of ETO with AML1); t(15;17) (acute promyolocytic leukemia; fusion of PML with RAR-α): t(12:15)(p13:q25) (Acute mycloid leukemia, congenital fibrosarcoma, secretory breast carcinoma; fusion of TEL with TrkC receptor), t(9;12)(p24;p13) (CML, ALL; fusion of JAK with TEL); t(12;21)(p12;q22) (ALL; fusion of TEL with AML1); t(11;18)(q21;q21) (MALT lymphoma; fusion of Bcl-2 with MLT); and t(1;11)(q42.1;q14.3) (schizophrenia).

Copy number variation analysis described herein can be used to diagnose prenatal conditions, e.g., fetal aneuploidy, e.g., trisomy 13, trisomy 18, or trisomy 21.

Determining the degree of degradation (fragmentation) of forensic genetic material can help determine what analyses can be successfully performed prior to wasting precious sample. Determining whether nucleic acids are linked or separated (fragmented) can be useful for determining an expected defect from perfect integer value copy number estimates due to random shearing of the DNA.

Detecting Deletions of Target Sequence

A method is provided for garnering linkage information through collocation. This method can be used to determine if there is a deletion of a target nucleic acid sequence, or for haplotyping CNV copies. A marker sequence (detected with, e.g., VIC labeled probe) can be outside but near a target sequence (detected with, e.g., a FAM-labeled probe), in a copy number variation region. A sample comprising nucleic acid can be partitioned into a plurality of spatially-isolated regions, and the marker and target nucleic acid sequences can be detected (e.g., through amplification and detection with probes). The collocation of the VIC (marker) and FAM (target) can be analyzed as depicted in FIG. 49. If VIC and FAM always colocalize in a partition, then there are likely no deletions of the target sequence (FIG. 49B). If there are partitions with VIC only that do not colocalize with FAM, this result suggests a deletion of the target sequence (FIG. 49A).

Storage of Digested Nucleic Acid

The length of storage of digested nucleic acid (e.g., DNA) can impact copy number variation measurements. Extended storage can cause reduction in the copy number estimated. For example, extended storage can result in nucleic acid degradation. The length of storage of a digested nucleic acid sample can be about, or less than, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 hrs. The length of storage of a digested nucleic acid sample can be about, or less than, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days. The length of storage of a digested nucleic acid sample can be about, or less than, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years.

In one embodiment, storing digested DNA at 4° C. for extended periods of time can affect the quality of a CN estimate (e.g., the a copy number estimate can become smaller over time, e.g., a sample with a target with an estimated CNV of 6.0, if stored for 3 weeks at 4° C., might yield a CNV of 5.7).

The storage temperature of a nucleic acid sample, (e.g., a digested nucleic acid sample) can be about, or less than 4, 0, −10, −20, −30, −40, −50, −60, −70, −80, −90, −100, −110, −120, −130, −140, −150, −160, −170, −180, −190, or −200° C.

In one embodiment, digested DNA can be stored in a buffer solution (e.g., 10 mM tris, pH 8.0).

In some embodiments, digested DNA can be lyophilized or dried (e.g., using a SpeedVac concentrator) for storage.

Impact of Nucleic Acid Length on CNV Analysis

The presence of long nucleic acids in a sample can affect copy number variation values even if target nucleic acid sequences are not linked (e.g., if they are on different chromosomes). Reduction of nucleic acid size in a sample by, e.g., restriction digestion, heat treatment, shearing, sonication, filtration, etc., can improve the results of a copy number variation experiment. Reduction in nucleic acid length can also improve target accessibility for PCR.

At high nucleic acid loads, reduction in the length of nucleic acids can be used to ensure consistent droplet formation in a droplet digital PCR experiment. At high nucleic acid loads with long nucleic acids, droplet formation can be reduced or prevented, and a stream can result. Nucleic acid length can be reduced by, e.g., sonication, heat treatment, restriction enzyme digest, filtering, or shearing.

Droplet digital PCR can be used to measure restriction enzyme efficiency and specificity.

This application incorporates by reference in their entirety for all purposes the following materials: U.S. Pat. No. 7,041,481, issued May 9, 2006; U.S. Patent Application Publication No. 2010/0173394 A1, published Jul. 8, 2010; and Joseph R. Lakowicz, PRINCIPLES OF FLUORESCENCE SPECTROSCOPY (2nd Ed. 1999).

Kits

Provided herein are kits for carrying out methods of the present disclosure. The kits can comprise one or more restriction enzymes, devices, buffers, reagents, and instructions for use. A kit can comprise a restriction enzyme, a buffer, a salt, and instructions for use. A kit can comprise one or more primers and one or more probes. A kit can comprise at least one restriction enzyme, four primers, and two probes. A kit can comprise at least one restriction enzyme, at least four primers, and at least one probe. A kit can comprise at least one restriction enzyme at least four primers, and at least two probes.

In some cases, a kit can comprise one or more plates, e.g., a plate for digital PCR. The plate can comprise a plurality of partitions. A kit can comprise about, more than, less than, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 plates. Each partition on a plate can comprise primers, e.g., a set of 4 primer pairs (8 primers) and/or a set of four probes. In some cases, each partition comprises a set of about, more than, less than, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 primer pairs. In some cases, each partition comprises about, more than, less than, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 sets of probes. A set of probes can comprise about, more than, less than, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 probes. In some cases, a kit comprising a plate, primers, and/or probes also comprises instructions.

Associated Technologies

Conventional techniques can be used in the methods described herein. Such conventional techniques can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press); Stryer, L. (1995) Biochemistry (4th Ed.) Freeman, New York: Gait, “Oligonucleotide Synthesis: A Practical Approach” 1984. IRL Press. London. Nelson and Cox (2000). Lehninger, (2004) Principles of Biochemistry 4^{th} Ed., W. H. Freeman Pub., New York, N.Y. and Berg et al. (2006) Biochemistry, 6th Ed., W. H. Freeman Pub., New York, N.Y., all of which are herein incorporated in their entirety by reference for all purposes.

Copy number variations can be detected by a variety of means including, e.g., fluorescence in situ hybridization, comparative genomic hybridization, array comparative genomic hybridization, virtual karyotyping with SNP arrays, and next-generation sequencing. Methods of determining copy number variation by digital PCR are described, for example, in U.S. Patent Application Publication No. 20090239308. Copy number variations can be detected by digital PCR by diluting nucleic acids. Copy number variations can be detected by digital PCR by using a nanofluidic chip (digital array) which can partition individual DNA molecules into separate reaction chambers (e.g., Fluidigm nanofluidic chip). Copy number variation can be detected by droplet digital PCR. The methods described herein can be used to confirm the result of a copy number variation analysis performed with one or more of the aforementioned techniques.

Next generation sequencing techniques that can be used to determine copy number variations include, e.g., DNA nanoball sequencing (using rolling circle replication to amplify small fragments of genomic DNA into DNA nanoballs) (used by, e.g., Complete Genomics), nanopore sequencing (used by, e.g., Oxford Nanopore Technologies, Genia Technologies, Nabsys) (Soni G. V. and Meller A. (2007) Clin. Chem. 53: 1996-2001), ion semiconductor sequencing (Ion Torrent Systems, Personal Genome Machine, Life Technologies) (U.S. Patent Application Publication No. 20090026082), SOLiD sequencing (sequencing by ligation; used by, e.g., Applied Biosystems), Illumina (Solexa) sequencing (using bridge amplification), 454 pyrosequencing (used by, e.g., Roche Diagnostics)(Margulies, M. et al. 2005 Nature, 437: 376-380), true single molecule sequencing (used by, e.g., Helicos) (Harris T. D. et al. (2008) Science 320: 106-109), sequencing using technology from Dover Systems (Polonator); or single molecule real-time sequencing (SMRT) used by Pacific Biosciences. The methods, compositions, and/or kits described herein can be used to follow-up on a CNV analysis performed by one of these methods. In some cases, the next generation sequencing technique is 454 sequencing (Roche) (see e.g., Margulies, M et al. (2005) Nature 437: 376-380). 454 sequencing can involve two steps. In the first step, DNA can be sheared into fragments of approximately 300-800 base pairs, and the fragments can be blunt ended. Oligonucleotide adaptors can then ligated to the ends of the fragments. The adaptors can serve as sites for hybridizing primers for amplification and sequencing of the fragments. The fragments can be attached to DNA capture beads, e.g., streptavidin-coated beads using, e.g., Adaptor B, which can contain 5′-biotin tag. The fragments can be attached to DNA capture beads through hybridization. A single fragment can be captured per bead. The fragments attached to the beads can be PCR amplified within droplets of an oil-water emulsion. The result can be multiple copies of clonally amplified DNA fragments on each bead. The emulsion can be broken while the amplified fragments remain bound to their specific beads. In a second step, the beads can be captured in wells (pico-liter sized; PicoTiterPlate (PTP) device). The surface can be designed so that only one bead fits per well. The PTP device can be loaded into an instrument for sequencing. Pyrosequencing can be performed on each DNA fragment in parallel. Addition of one or more nucleotides can generate a light signal that can be recorded by a CCD camera in a sequencing instrument. The signal strength can be proportional to the number of nucleotides incorporated.

Pyrosequencing can make use of pyrophosphate (PPi) which can be released upon nucleotide addition. PPi can be converted to ATP by ATP sulfurylase in the presence of adenosine 5′ phosphosulfate. Luciferase can use ATP to convert luciferin to oxyluciferin, and this reaction can generate light that can be detected and analyzed. The 454 Sequencing system used can be GS FLX+ system or the GS Junior System.

In some embodiments, the next generation sequencing technique is SOLiD technology (Applied Biosystems; Life Technologies). In SOLiD sequencing, genomic DNA can be sheared into fragments, and adaptors can be attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations can be prepared in microreactors containing beads, primers, template, and PCR components. Following PCR, the templates can be denatured and beads can be enriched to separate the beads with extended templates. Templates on the selected beads can be subjected to a 3′ modification that permits bonding to a glass slide. A sequencing primer can bind to adaptor sequence. A set of four fluorescently labeled di-base probes can compete for ligation to the sequencing primer. Specificity of the di-base probe can be achieved by interrogating every first and second base in each ligation reaction. The sequence of a template can be determined by sequential hybridization and ligation of partially random oligonucleotides with a determined base (or pair of bases) that can be identified by a specific fluorophore. After a color is recorded, the ligated oligonucleotide can be cleaved and removed and the process can be then repeated. Following a series of ligation cycles, the extension product can be removed and the template can be reset with a primer complementary to the n-1 position for a second round of ligation cycles. Five rounds of primer reset can be completed for each sequence tag. Through the primer reset process, most of the bases can be interrogated in two independent ligation reactions by two different primers. Up to 99.99% accuracy can be achieved by sequencing with an additional primer using a multi-base encoding scheme. In some cases, the next generation sequencing machine is a 5500 W Series Genetic Analysis System.

In some cases, the next generation sequencing technique is SOLEXA sequencing (ILLUMINA sequencing). ILLUMINA sequencing can be based on the amplification of DNA on a solid surface using fold-back PCR and anchored primers. ILLUMINA sequencing can involve a library preparation step. Genomic DNA can be fragmented, and sheared ends can be repaired and adenylated. Adaptors can be added to the 5′ and 3′ ends of the fragments. The fragments can be size selected and purified. ILLUMINA sequence can comprise a cluster generation step. DNA fragments can be attached to the surface of flow cell channels by hybridizing to a lawn of oligonucleotides attached to the surface of the flow cell channel. The fragments can be extended and clonally amplified through bridge amplification to generate unique clusters. The fragments become double stranded, and the double stranded molecules can be denatured. Multiple cycles of the solid-phase amplification followed by denaturation can create several million clusters of approximately 1,000 copies of single-stranded DNA molecules of the same template in each channel of the flow cell. Reverse strands can be cleaved and washed away. Ends can be blocked, and primers can by hybridized to DNA templates. ILLUMINA sequencing can comprise a sequencing step. Hundreds of millions of clusters can be sequenced simultaneously. Primers, DNA polymerase and four fluorophore-labeled, reversibly terminating nucleotides can be used to perform sequential sequencing. All four bases can compete with each other for the template. After nucleotide incorporation, a laser can be used to excite the fluorophores, and an image is captured and the identity of the first base is recorded. The 3′ terminators and fluorophores from each incorporated base are removed and the incorporation, detection and identification steps are repeated. A single base can be read each cycle. In some embodiments, a HiSeq system (e.g., HiSeq 2500, HiSeq 1500, HiSeq 2000, or HiSeq 1000) is used for sequencing. In some embodiments, a MiSeq personal sequencer is used. In some embodiments, a Genome Analyzer IIx is used.

In some cases, the next generation sequencing technique comprises real-time (SMRT™) technology by Pacific Biosciences. In SMRT, each of four DNA bases can be attached to one of four different fluorescent dyes. These dyes can be phospholinked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zeptoliters (10^{˜21} liters) can be created. The tiny detection volume can provide 1000-fold improvement in the reduction of background noise. Detection of the corresponding fluorescence of the dye can indicate which base was incorporated. The process can be repeated. In some cases, a PacBio RS II is used for next generation sequencing.

In some cases, the next generation sequencing is nanopore sequencing (See e.g., Soni G V and Meller A. (2007) Clin Chem 53: 1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridlON system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or SlO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi:10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some embodiments, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

In some cases, nanopore sequencing technology from GENIA is used. An engineered protein pore can be embedded in a lipid bilayer membrane. “Active Control” technology can be used to enable efficient nanopore-membrane assembly and control of DNA movement through the channel. In some embodiments, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some embodiments, the nanopore sequencing technology is from IBM/Roche. A electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

In some cases, the next generation sequencing comprises ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an ION PROTON™ Sequencer is used to sequence nucleic acid. In some cases, an ION PGM™ Sequencer is used.

In some cases, the next generation sequencing is DNA nanoball sequencing (as performed, e.g., by Complete Genomics; see e.g., Drmanac et al. (2010) Science 327: 78-81; Carnevali et al., J Comp Biol 2012). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template. Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamehtyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

In some cases, the next generation sequencing technique is Helicos True Single Molecule Sequencing (tSMS) (see e.g., Harris T. D. et al. (2008) Science 320:106-109). In the tSMS technique, a DNA sample can be cleaved into strands of approximately 100 to 200 nucleotides, and a polyA sequence can be added to the 3′ end of each DNA strand. Each strand can be labeled by the addition of a fluorescently labeled adenosine nucleotide. The DNA strands can then be hybridized to a flow cell, which can contain millions of oligo-T capture sites immobilized to the flow cell surface. The templates can be at a density of about 100 million templates/cm². The flow cell can then be loaded into an instrument, e.g., HELISCOPE™ sequencer, and a laser can illuminate the surface of the flow cell, revealing the position of each template. A CCD camera can map the position of the templates on the flow cell surface. The template fluorescent label can then be cleaved and washed away. The sequencing reaction can begin by introducing a DNA polymerase and a fluorescently labeled nucleotide. The oligo-T nucleic acid can serve as a primer. The DNA polymerase can incorporate the labeled nucleotides to the primer in a template directed manner. The DNA polymerase and unincorporated nucleotides can be removed. The templates that have directed incorporation of the fluorescently labeled nucleotide can be detected by imaging the flow cell surface. After imaging, a cleavage step can remove the fluorescent label, and the process can be repeated with other fluorescently labeled nucleotides until a desired read length is achieved. Sequence information can be collected with each nucleotide addition step. The sequencing can be asynchronous. The sequencing can comprise at least 1 billion bases per day or per hour.

In some cases, the sequencing technique can comprise paired-end sequencing in which both the forward and reverse template strand can be sequenced. In some cases, the sequencing technique can comprise mate pair library sequencing. In mate pair library sequencing, DNA can be fragments, and 2-5 kb fragments can be end-repaired (e.g., with biotin labeled dNTPs). The DNA fragments can be circularized, and non-circularized DNA can be removed by digestion. Circular DNA can be fragmented and purified (e.g., using the biotin labels). Purified fragments can be end-repaired and ligated to sequencing adaptors.

In some cases, a sequence read is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 bases.

In some embodiments, a sequence read is more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 bases.

In some embodiments, a sequence read is less than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 bases.

In some embodiments, a sequence read is at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 bases.

In some cases, a sequence read is about 10 to about 50 bases, about 10 to about 100 bases, about 10 to about 200 bases, about 10 to about 300 bases, about 10 to about 400 bases, about 10 to about 500 bases, about 10 to about 600 bases, about 10 to about 700 bases, about 10 to about 800 bases, about 10 to about 900 bases, about 10 to about 1000 bases, about 10 to about 1500 bases, about 10 to about 2000 bases, about 50 to about 100 bases, about 50 to about 150 bases, about 50 to about 200 bases, about 50 to about 500 bases, about 50 to about 1000 bases, about 100 to about 200 bases, about 100 to about 300 bases, about 100 to about 400 bases, about 100 to about 500 bases, about 100 to about 600 bases, about 100 to about 700 bases, about 100 to about 800 bases, about 100 to about 900 bases, about 100 to about 1000 bases, about 200 to about 400 bases, or about 150 to about 300 bases.

The number of sequence reads from a sample can be about 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The number of sequence reads from a sample can be more than 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The number of sequence reads from a sample can be less than 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The number of sequence reads from a sample can be at least 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The number of reads per run can be about 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The number of reads per run can be more than 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The number of reads per run can be more than less than 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The number of reads per run can be at least 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The depth of sequencing of a sample can be about 1×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, 110×, 120×, 130×, 140×, 150×, 160×, 170×, 180×, 190×, 200×, 300×, 400×, 500×, 600×, 700×, 800×, 900×, 1000×, 1500×, 2000×, 2500×, 3000×, 3500×, 4000×, 4500×, 5000×, 5500×, 6000×, 6500×, 7000×, 7500×, 8000×, 8500×, 9000×, 9500×, or 10,000×.

The depth of sequencing of a sample can be more than 1×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, 110×, 120×, 130×, 140×, 150×, 160×, 170×, 180×, 190×, 200×, 300×, 400×, 500×, 600×, 700×, 800×, 900×, 1000×, 1500×, 2000×, 2500×, 3000×, 3500×, 4000×, 4500×, 5000×, 5500×, 6000×, 6500×, 7000×, 7500×, 8000×, 8500×, 9000×, 9500×, or 10,000×.

The depth of sequencing of a sample can be less than 1×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, 110×, 120×, 130×, 140×, 150×, 160×, 170×, 180×, 190×, 200×, 300×, 400×, 500×, 600×, 700×, 800×, 900×, 1000×, 1500×, 2000×, 2500×, 3000×, 3500×, 4000×, 4500×, 5000×, 5500×, 6000×, 6500×, 7000×, 7500×, 8000×, 8500×, 9000×, 9500×, or 10,000×.

The depth of sequencing of a sample can be at least 1×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, 110×, 120×, 130×, 140×, 150×, 160×, 170×, 180×, 190×, 200×, 300×, 400×, 500×, 600×, 700×, 800×, 900×, 1000×, 1500×, 2000×, 2500×, 3000×, 3500×, 4000×, 4500×, 5000×, 5500×, 6000×, 6500×, 7000×, 7500×, 8000×, 8500×, 9000×, 9500×, or 10,000×.

The depth of sequencing of a sample can about 1× to about 5×, about 1× to about 10×, about 1× to about 20×, about 5× to about 10×, about 5× to about 20×, about 5× to about 30×, about 10× to about 20×, about 10× to about 25×, about 10× to about 30×, about 10× to about 40×, about 30× to about 100×, about 100× to about 200×, about 100× to about 500×, about 500× to about 1000×, about 1000× to about 2000×, about 1000× to about 5000×, or about 5000× to about 10,000×. Depth of sequencing can be the number of times a sequence (e.g., a genome) is sequenced. In some embodiments, the Lander/Waterman equation is used for computing coverage. The general equation can be: C=LN/G, where C=coverage; G=haploid genome length; L=read length; and N=number of reads.

In some cases, different barcodes can be added to polynucleotides in different samples (e.g., by using primers or adaptors), and the different samples can be pooled and analyzed in a multiplexed assay. The barcode can allow the determination of the sample from which a polynucleotide originated. A barcode can be on an adaptor that is attached to a polynucleotide. An adaptor can be single stranded, double stranded, Y-shaped (e.g., comprising a paired portion on one end and un-paired portion on the other end), and/or have the ability to form a stem loop. The barcode can be on the single stranded or double stranded portion of the adaptor. In other cases, a barcode can be an endogenous sequence on a polynucleotide. A barcode can be about 1, 2, 3, 4, 5, 6, 7, 8, 9, 10, 11, 12, 13, 14, 15, 16, 17, 18, 19, or 20 bases. A barcode can be more than 1, 2, 3, 4, 5, 6, 7, 8, 9, 10, 11, 12, 13, 14, 15, 16, 17, 18, 19, or 20 bases. A barcode can be less than 1, 2, 3, 4, 5, 6, 7, 8, 9, 10, 11, 12, 13, 14, 15, 16, 17, 18, 19, or 20 bases. A barcode can be at least 1, 2, 3, 4, 5, 6, 7, 8, 9, 10, 11, 12, 13, 14, 15, 16, 17, 18, 19, or 20 bases. The number of barcoded samples that can be pooled can be more than 5, 10, 15, 20, 25, 30, 35, 40, 45, 50, 60, 70, 80, 90, or 100.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5.

Unless defined otherwise, all technical and scientific terms used herein have the same meaning as commonly understood by one of ordinary skill in the art. Although any methods and materials similar or equivalent to those described herein can also be used in the practice or testing of the present methods, representative illustrative methods and materials are now described.

### EXAMPLES

### Example 1

### Directional Mapping

FIG. 1 illustrates an example of a 4-plex linkage assay for determining arrangement of loci on a chromosome and directional mapping of genomic rearrangements. The white bar (102) is a schematic of a reference chromosome. Loci B1, B2, B3, and B4 comprise different nucleic acid sequences that are recognized by different probe that comprise the same first label. Loci G1, G2, G3, and G4 are also different sequences recognized by different probes comprising the same second label. Loci O1, O2, O3, and O4 are also different sequences recognized by different probes comprising the same third label. A control chromosome (104), illustrated nine times in FIG. 1, comprises a sequence R1 that is recognized by a probe comprising a fourth label. Because the probe comprising the fourth label does not anneal to the reference chromosome, no linkage should exist between the marker on the control chromosome (104) and any of the markers on the reference chromosome (102). The number above each control chromosome (104) represents a number of a 4-plex assay (set of R1, and B, G, and O sequences). Nine 4-plex linkage assays are shown (1-9). Distances of the loci from locus B1 on the reference chromosome are illustrated. For example, marker G1 is 50 kb from locus B1, and locus O1 is 100 kb from locus B1.

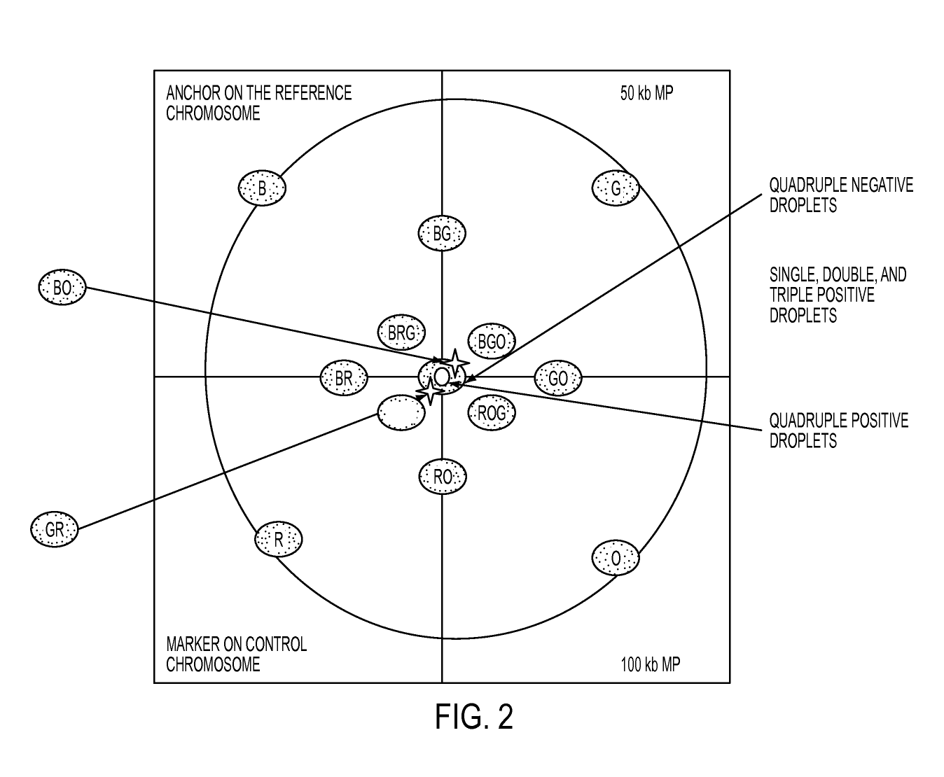

FIG. 2 illustrates possible combinations of labels in a partition in a digital PCR experiment. FIG. 2 illustrates a four-dimensional droplet amplitude plot drawn as a two-dimensional figure, where in a 4-plex linkage assay, each of the four assays fluoresces in a different channel, shown here as a quadrant. The upper left hand quadrant represents the anchor loci (B). The upper right hand quadrant represents loci 50 kb away from the anchor sequences (B). The bottom right quadrant represents markers 100 kb away from the anchor loci (B). The bottom left quadrant represents the loci on the control chromosome. At very low concentrations, it is expected that the same partition will not contain the triple or quadruple signals. Because R1 is on a different chromosome than the reference chromosome, no linkage is expected between R1 and the B, G, or O markers. Some degree of linkage is expected between B, G, and O.

In a digital experiment, linkage can depend on the degree of fragmentation of the nucleic acid sample. In the instant example, it is assumed that loci less than or equal to 25 kb apart demonstrate 100% linkage; loci 50 kb apart demonstrate 66% linkage; loci 75 kb apart demonstrate 33% linkage; loci 100 kb apart demonstrate 10% linkage, and loci greater than 100 kb apart demonstrate 0% linkage. Each four-plex assay can provide copy number estimates of each of the targets.

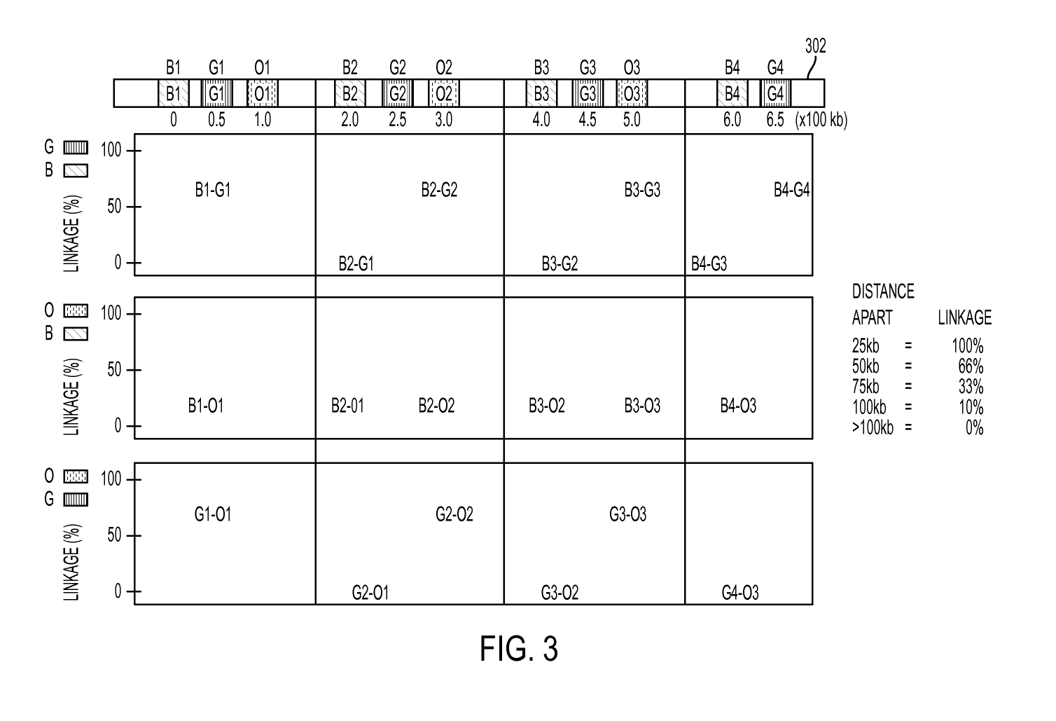

FIG. 3 illustrates a hypothetical analysis of a reference genome using multiple 4-plex assays. A digital PCR experiment is performed in which probes with four different labels are used. A schematic chromosome (302) illustrates different loci B1, G1, O1, B2, G2, O2, B3, G3, O3, B4, and G4. In the experiment whose hypothetical results are illustrated in the top row, different probes with the same label are designed to anneal to sequences B1, B2, B3, and B4, and different probes with the same label are designed to anneal to sequences G1, G2, G3, and G4. Based on the assumption above regarding the fragmentation frequency of a genomic DNA sample, B1 and G1, being 50 kb apart, are linked at a frequency of 66%, B2 and G2 are linked at a frequency of 66%, B3 and G3 are linked at a frequency of 66%, and B4 and G4 are linked at a frequency of 66%. B2 and G1 are separated by 150 kb and are linked at a frequency of 0%. B3-G2 and B4-G3 are linked at a frequency of 0% as well. All B1-O1, B2-O1, B2-O2, B3-O2, B3-O3, and B4-O4 are each separated by 100 kb and are linked at a frequency of 10%. G1-O1, G2-O2, and G3-O3 are each separated by 50 kb and are linked at a frequency of 66%. G2-O1, G3-O2, and G4-O3 are separated by 150 kb and are linked at a frequency of 0%. The strategy illustrate allows for confirmation of findings in separate wells. Every assay, when paired with the assay for the control sequence R1, should have a linkage of 0%.

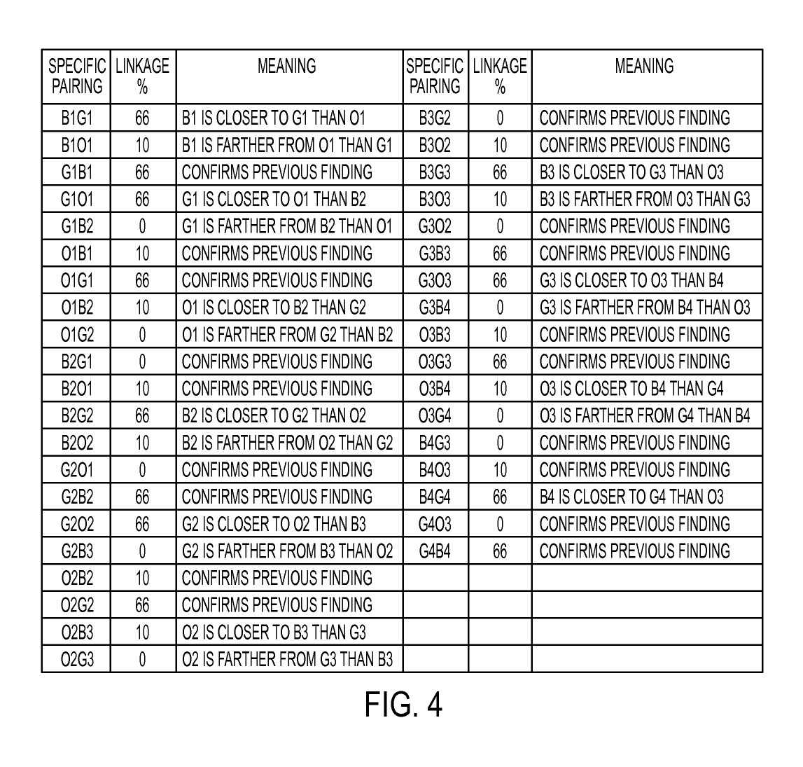

FIG. 4 illustrates interpretations of the hypothetical results of the digital PCR experiment shown in FIG. 3. The identity of the primers used and the label of the probes to detect amplified product is known. By analyzing the percentage linkage of specific pairs of loci, the order of the loci on the reference chromosome can be deduced.

FIG. 5 illustrates results of a hypothetical digital PCR experiment of a sample comprising a chromosome with a genomic rearrangement (506). Pairs of loci that show changes in frequency of linkage relative to the reference chromosome (502) are boxed in FIG. 5. For example, a rearrangement has occurred between O2 and G2. Now, B2 and G2 are separated by 100 kb and show a linkage frequency of 10%, whereas on the reference chromosome B2 and G2 are separated by 50 kb and show a linkage frequency of 66%. B3 and G2 are separated by 100 kb on the rearranged chromosome and show a linkage frequency of 10%. G4 and O3 are also rearranged. However, B4 and G4 are still separated by 50 kb and have a linkage frequency of 66%. B2 and O2 are separated by 50 kb and show a linkage frequency of 66%. B3 and O2 are now separated by 150 kb and have a linkage frequency of 0%. B3 and O3 are now separated by 250 kb and show a linkage frequency of 0%. B4 and O3 are now separated by 50 kb and display a linkage frequency of 66%. G3 and O3 are now separated by 200 kb and show a linkage frequency of 0%. G4 and O3 are separated by 100 kb and show a linkage frequency of 10%.

FIG. 6 illustrates an analysis of the hypothetical data in FIG. 5. The column with percent linkage shows linkage numbers for the rearranged chromosome and illustrates differences with the linkage frequency compared to the reference chromosome. Every assay when paired with the control chromosome should yield 0% linkage, which can confirm random distribution in the digital PCR sample.

### Example 2

### Algorithm for Determining Fragmentation

In this example, two different types of target nucleic acid are being analyzed. One is being detected with a FAM probe and one is being detected with VIC. Assume that the two target nucleic acid sequences are on the same polynucleotide. In a sample, there can be three types of DNA fragments: 1) Fam-Vic together (not chopped), 2) Fam fragment, and 3) Vic fragment. Some probabilities are observed (counts in FAM−VIC cross plot), and the goal is to infer the concentrations. Forward is done first. Given concentrations, counts are computed. Then to do inverse, try out different values of concentrations and select one which gives actual counts.

N = 20000;
A = 10000;
B = 20000;
AB = 10000; % Joined together
cA = A/N;
cB = B/N;
cAB = AB/N;
fprintf(1, ′%f%f%f\n′, cAB, cA, cB);
pA = 1 − exp(−cA);
pB = 1 − exp(−cB);
pAB = 1 − exp(−cAB);
%A is X and B is Y in cross plot
p(2,1) = (1 − pA) * (1 − pB) * (1 − pAB); % Bottom left
p(2,2) = pA * (1 − pB) * (1 − pAB); % Bottom right
p(1,1) = (1 − pA) * pB * (1 − pAB); % Top Left
p(1,2) = 1 − p(2,1) − p(2,2) − p(1,1); % Top Right
disp(round(p * N));
% Also compute marginals directly
cAorAB = (A + AB)/N; % = c_A + c_AB;
cBorAB = (B + AB)/N; % = c_B + c_AB;
pAorAB = 1 − exp(−cAorAB); % Can be computed from p too
pBorAB = 1 − exp(−cBorAB);
% Inverse
H = p * N; % We are given some hits
%H = [0 8000;2000 0];
% Compute prob
estN = sum(H(:));
i_p = H/estN;
i_pAorAB = i_p(1,2) + i_p(2,2);
i_pBorAB = i_p(1,1) + i_p(1,2);
i_cAorAB = −log(1 − i_pAorAB);
i_cBorAB = −log(1 − i_pBorAB);
maxVal = min(i_cAorAB, i_cBorAB);
delta = maxVal/1000;
errArr = [ ];
gcABArr = [ ];
for gcAB = 0:delta:maxVal
gcA = i_cAorAB − gcAB;
gcB = i_cBorAB − gcAB;
gpA = 1 − exp(−gcA);
gpB = 1 − exp(−gcB);
gpAB = 1 − exp(−gcAB);
gp(2,1) = (1 − gpA) * (1 − gpB) * (1 − gpAB); % Bottom left
gp(2,2) = gpA * (1 − gpB) * (1 − gpAB); % Bottom right
gp(1,1) = (1 − gpA) * gpB * (1 − gpAB); % Top Left
gp(1,2) = 1 − gp(2,1) − gp(2,2) − gp(1,1); % Top Right
gH = gp * estN;
err = sqrt(sum((H(:) − gH(:)).\hat{ }2));
errArr = [errArr; err];
gcABArr = [gcABArr; gcAB];
end
figure, plot(gcABArr, errArr);
minidx = find(errArr == min(errArr(:)));
minidx = minidx(1);
estAB = gcABArr(minidx);
estA = i_cAorAB − estAB;
estB = i_cBorAB − estAB;
fprintf(1, ′%f%f%f\n′, estAB, estA, estB);
gpA = 1 − exp(−estA);
gpB = 1 − exp(−estB);
gpAB = 1 − exp(−estAB);
gp(2,1) = (1 − gpA) * (1 − gpB) * (1 − gpAB); % Bottom left
gp(2,2) = gpA * (1 − gpB) * (1 − gpAB); % Bottom right
gp(1,1) = (1 − gpA) * gpB * (1 − gpAB); % Top Left
gp(1,2) = 1 − gp(2,1) − gp(2,2) − gp(1,1); % Top Right
gH = gp * estN;
disp(round(gH));
% Confirm the results using simulation
numMolA = round(estA * estN);
numMolB = round(estB * estN);
numMolAB = round(estAB * estN);
A = unique(randsample(estN, numMolA, 1));
B = unique(randsample(estN, numMolB, 1));
AB = unique(randsample(estN, numMolAB, 1));
U = 1:estN;
notA = setdiff(U, A);
notB = setdiff(U, B);
notAB = setdiff(U, AB);
AorBorAB = union(A, union(B, AB));
none = setdiff(U, AorBorAB);
simcount(2,1) = length(none);
simcount(2,2) = length(intersect(A, intersect(notB, notAB)));
simcount(1,1) = length(intersect(B, intersect(notA, notAB)));
simcount(1,2) = length(AorBorAB) − simcount(2,2) − simcount(1,1);
disp(simcount);

### Example 3

### Milepost Assay Analysis-Probability of Fragmentation

Problem Statement

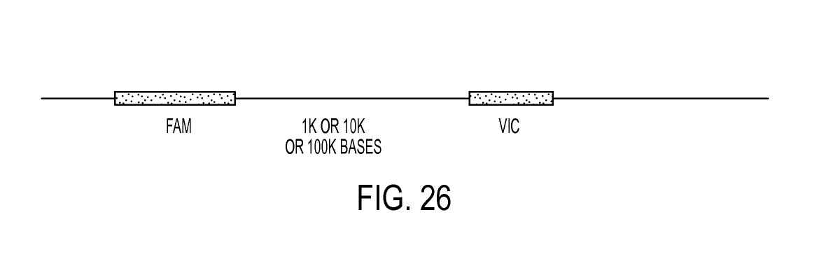

If two different loci are on different molecules, there can be two species (corresponding to FAM and VIC probes). If the different loci are on the same molecule, there can be three species-fragmented FAM, fragmented VIC, and linked FAM−VIC. (See FIG. 26)

There are two dyes, so there can be ambiguity. There is a need to compute concentrations of all three species.

Algorithm: Get 2×2 table of FAM versus VIC counts. Compute concentration of fragmented FAM and linked FAM−VIC as if there is 1 species. Compute concentration of fragmented VIC and linked FAM−VIC as if there is I species. Try out different concentrations of linked FAM−VIC (from which concentration of fragmented FAM and VIC can be found), and find the best fit of the probability table with the observed counts:

| | FAM− | FAM+ |
|---|---|---|
| VIC+ | (1 − f) v (1 − c) | 1 − sum of others |
| VIC− | (1 − f) (1 − v) (1 − c) | f (1 − v) (1 − c) |

### Probability of Fragmentation (in %)

| | | | |
|---|---|---|---|
| 1k Uncut | 6 | 6 | — |
| 10K Uncut | 29.4 | 29.8 | 29.5 |
| 100K Uncut | 98.7 | 97.7 | 99.9 |
| 1K Syringe | 11.4 | 11.1 | 11 |
| 10K Syringe | 87.2 | 89.9 | 91.7 |
| 100K Syringe | 100 | 100 | 100 |
| 1K Hae III | 100 | 100 | 100 |

Next steps can include to see if a closed formula can be easily derived and/or to integrate with QTools.

### Example 4

### Fragmentation Analysis

Using ddPCR, duplex reaction targeting two genomic loci can be performed, two genes on a common chromosome for example. The droplets can be categorized into four populations according to their fluorescence (FAM+NIC+, FAM+/VIC−, FAM−/VIC+, and FAM−/VIC−). By comparing the number of droplets with these populations, it is possible to determine the frequency at which targets co-segregate to the same droplet. Using Poisson statistics, the percentage of species that are actually linked to one another can be estimated, versus instances where two separated copies are in the same droplet by chance.

An assay is designed in which a locus is 1K, 3K, 10K, 33K and 100K away from a common reference (RPP30). Studies in which two loci are separated by 1K, 10K and 100K have been performed. By processing uncut (not restriction enzyme digested) DNA with these three duplexes (or just one duplex), and counting the four different populations of droplets, statistical analysis can be used to assess the fragmentation status of the genetic material. These data can be used to help explain why 95% confidence limits for copy number variation studies do not always span the integer value.

### Example 5

### Algorithm for Computation of DNA Fragmentation or for Digital PCR Multiplexing

Total Fragmentation Between Targets

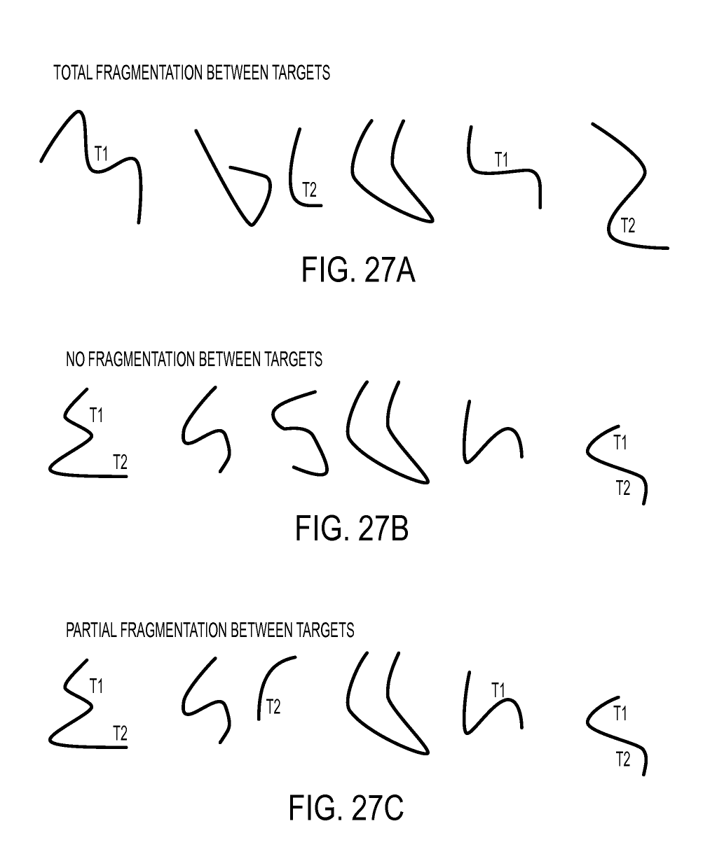

Two DNA targets T1 and T2 correspond to two dyes, FAM and VIC, respectively. In this example, T1 and T2 are always on separate DNA fragments. The number of DNA fragments with T1 and T2 targets is M1 and M2, respectively. See FIG. 27A.

In a digital PCR experiment with multiple partitions, the counts of FAM and VIC positive partitions is N1 and N2, respectively. N1 and N2 will be smaller than M1 and M2, respectively, because there can be multiple DNA fragments in a partition. The total number of partitions is N. The counts of partitions as expected are shown in Table 2.

TABLE 2
Counts of partitions.

| | VIC Negative | VIC Positive | Total |
|---|---|---|---|
| FAM Positive | N1*(N − N2)/N | N1*N2/N | N1 |
| FAM Negative | (N − N1)*(N − N2)/N | (N − N1)*N2/N | N − N1 |
| Total | N − N2 | N2 | N |

If the probability of observing a FAM positive partition is denoted as p1=N1/N, and the probability of observing a VIC positive partition is denoted as p2=N2/N, then the corresponding probability table is Table 3.

TABLE 3
Probability table.

| | VIC Negative | VIC Positive | Probability |
|---|---|---|---|
| FAM Positive | p1*(1 − p2) | p1*p2 | p1 |
| FAM Negative | (1 − p1)*(1 − p2) | (1 − p1)*p2 | 1 − p1 |
| Total | 1 − p2 | P2 | 1 |

In this case, 100% fragmentation between T1 and T2 exists.

The number of T1 and T2 molecules, M1 and M2, respectively, can be computed as follows:

M1=−N log(1−p1)

M2=−N log(1−p2)

(Given N digital partitions in which P are positive, the number of molecules is M=−N log(1−P/N))

No Fragmentation Between Targets

If both T1 and T2 are always on the same DNA fragment, they are linked (perhaps because their loci are quite close to each other on the same part of a chromosome and restriction enzyme digest did not digest between T1 and T2). See FIG. 27B. Therefore, N1=N2.

TABLE 4
Counts of partitions.

| | VIC Negative | VIC Positive | Total |
|---|---|---|---|
| FAM Positive | 0 | N1 | N1 |
| FAM Negative | N − N1 | 0 | N − N1 |
| Total | N − N1 | N1 | N |

TABLE 5
Probability table.

| | VIC Negative | VIC Positive | Probability |
|---|---|---|---|
| FAM Positive | 0 | p1 | p1 |
| FAM Negative | 1 − p1 | 0 | 1 − p1 |
| Total | 1 − p1 | p1 | 1 |

In this case, 0% fragmentation exists.

The number of T1 and T2 molecules can be computed as follows, where p1=N1/N:

M1=−N log(1−p1)

M2=−N log(1−p1)

Partial Fragmentation

In an intermediate situation, T1 and T2 are linked on some fragments, but also happen to be on separate fragments. See FIG. 27C.

If there are M3 molecules of linked T1 and T2 fragments, M1 molecules of separate T1 fragments, and M2 molecules of separate T2 fragments, the following table of counts of partitions can be made:

TABLE 6
Counts of partitions.

| | VIC Negative | VIC Positive | Total |
|---|---|---|---|
| FAM Positive | N01 | N11 | N1 |
| FAM Negative | N00 | N10 | N − N1 |
| Total | N − N2 | N2 | N |

If M1=M2=M3, then there is 50% fragmentation, because 50% of linked molecules were fragmented into separate fragments and 50% remained intact.

### Example 6

### Assessing DNA Quality in Plasma with the Milepost Assay

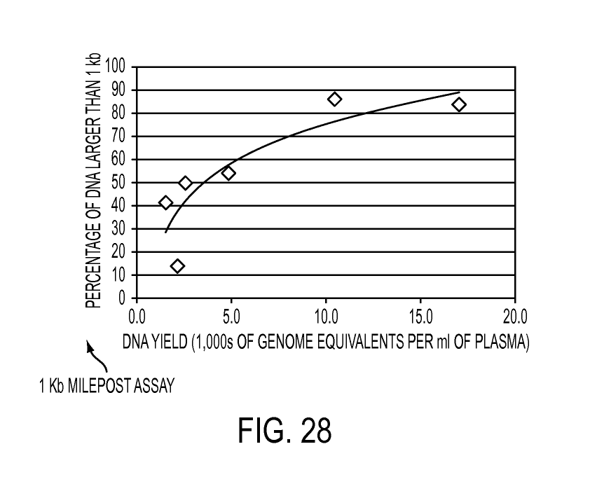

It appears that in samples with higher DNA yield, the extra DNA is predominantly large in size. As shown in FIG. 28 when the DNA yield is around 2 kGE (Genome equivalents)/ml, roughly half of the DNA is less than 1 Kb in size; when the yield is extremely high (10 kGE/ml or more), 90% of the DNA is larger than 1 Kb. This suggests that small DNA is relatively constant in concentration. This suggests further that higher DNA yields are due to contamination from cellular DNA.

### Example 7

### Haplotyping Through Collocation

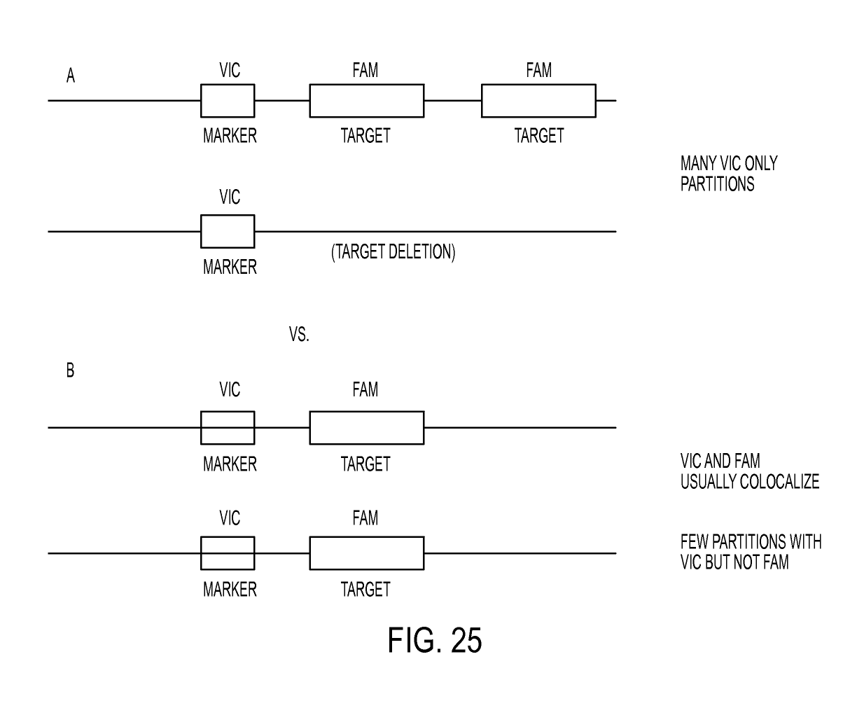

A method is provided for garnering haplotyping information through collocation. This method can be used to determine if there is a deletion of a target nucleic acid sequence. A marker sequence (detected with, e.g., VIC labeled probe) can be outside but near a target sequence (detected with, e.g., a FAM-labeled probe), in a copy number variation region. A sample comprising nucleic acid can be partitioned into a plurality of spatially-isolated regions, and the marker and target nucleic acid sequences can be detected (e.g., through amplification and detection with probes). The collocation of the VIC (marker) and FAM (target) can be analyzed as depicted in FIG. 25. If VIC and FAM always colocalize in a partition, then there are likely no deletions of the target sequence (FIG. 25B). If there are partitions with VIC only that do not colocalize with FAM, this result suggests a deletion of the target sequence (FIG. 25A).

### Example 8

### Linkage Analysis

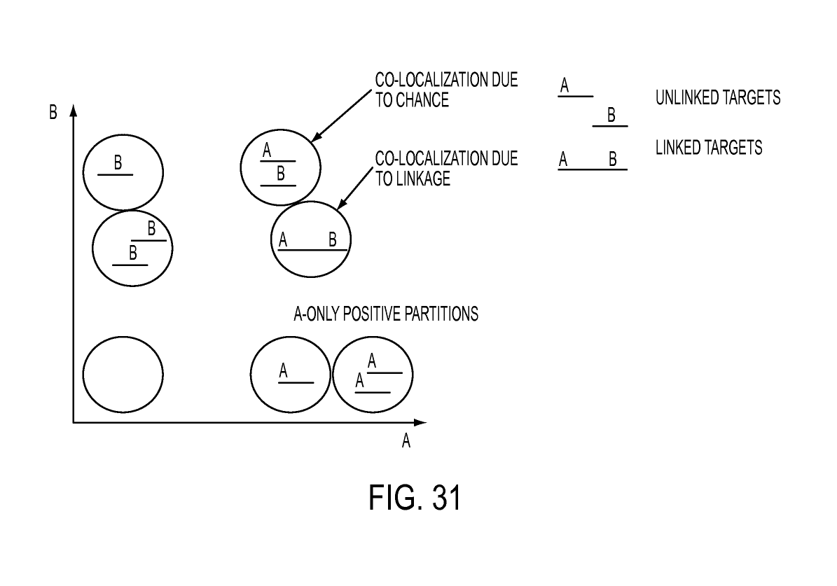

In this example, two targets, A and B, are to be detected on two different channels with probes with different labels. Depending on what kind of molecules are present initially in each partition (droplet) the partitions can appear as positive or negative on each channel. The double positive partitions may be due to colocalization due to chance or due to linkage (A and B are physically on the same molecule) (FIG. 31).

N0—number of double negative partitions

Na—number of A-only positive partitions

Nb—number of B-only positive partitions

N1—number of double positive partitions

N_ch—number of double positive partitions due to chance

N_1—number of double positive partitions due to linkage

N1 is directly observed, and N_ch and N_1 can be deduced from other data

N1=N_ch+N_1; N_ch=Na*Nb/N0

N_1=N−Na*Nb/N0

### Example 9

### Determining Distances

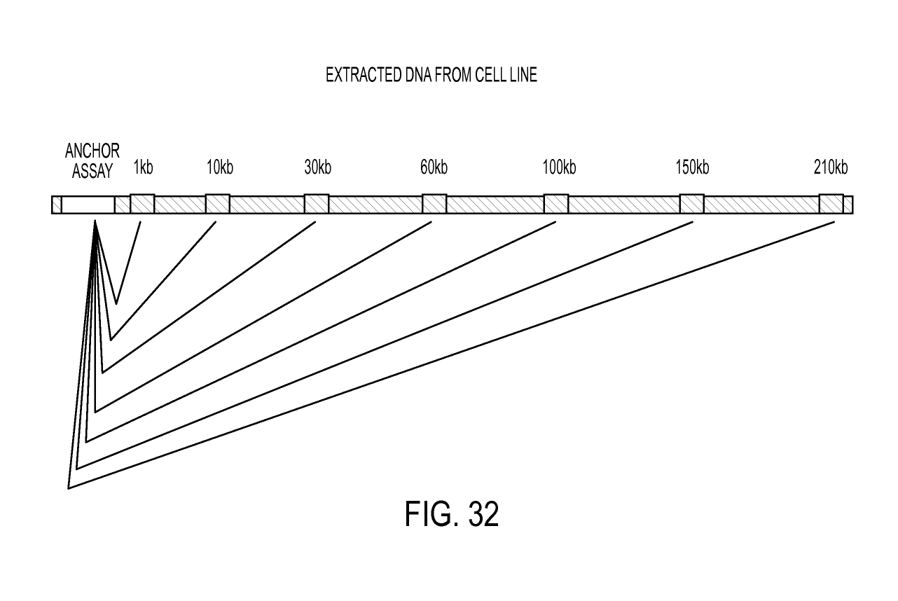

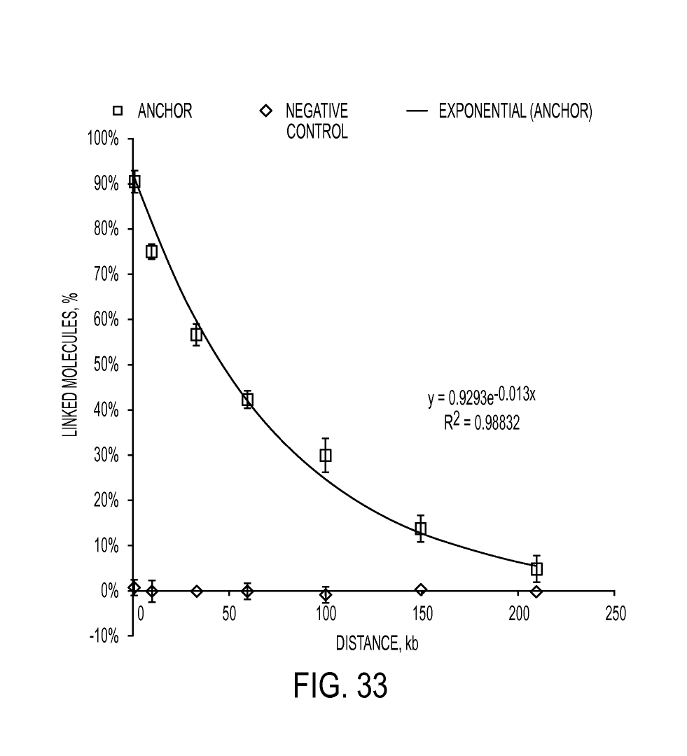

Distances among loci can be assessed, e.g., an assay can be performed to determine that a locus A is farther from locus B than locus C. To measure distance, the linkage frequencies can be compared to a sample standard. For example, a series of “mile” marker duplex assays can be used. In a mile marker experiment, an anchor locus can be targeted with a probe, e.g., labeled with HEX (a HEX assay), and markers at increasing distance from this anchor point can all targeted using a unique probe (e.g., a FAM probe) (FAM assay) (See e.g., FIG. 32). To test linkage at different distances, DNA can be extracted from an immortalized B-lymphocyte cell line, and the DNA can be screened using seven mile marker duplex assays. By assembling a series of duplex assays, and measuring the percent of linked loci in each duplex assay, an equation can be generated that describes a curve that fits the data. The relationship can be an exponential relationship (see e.g., FIG. 33). FIG. 33 illustrates a percentage of linked molecules on the Y axis as a function of the distance separating the mile markers from the anchor sequence on the X-axis. Across 3 extractions, data is fit to an exponential model with a uniform DNA fragmentation probability per kb. Linkage can be measured out to approximately 300 kb in a single partition (e.g., well). The control for no linkage, an assay targeting a different chromosome, shows no significant linkage for any of the mile markers.

With the same sample (in some cases, with no freeze-thaw differences), a chromosome mapping experiment can be performed. The percent linkage found for loci can be compared to the equation for the line, providing an estimate for distance between loci. Fragmentation rate between chromosomes can be preserved and can be independent of specific nucleotide sequence.

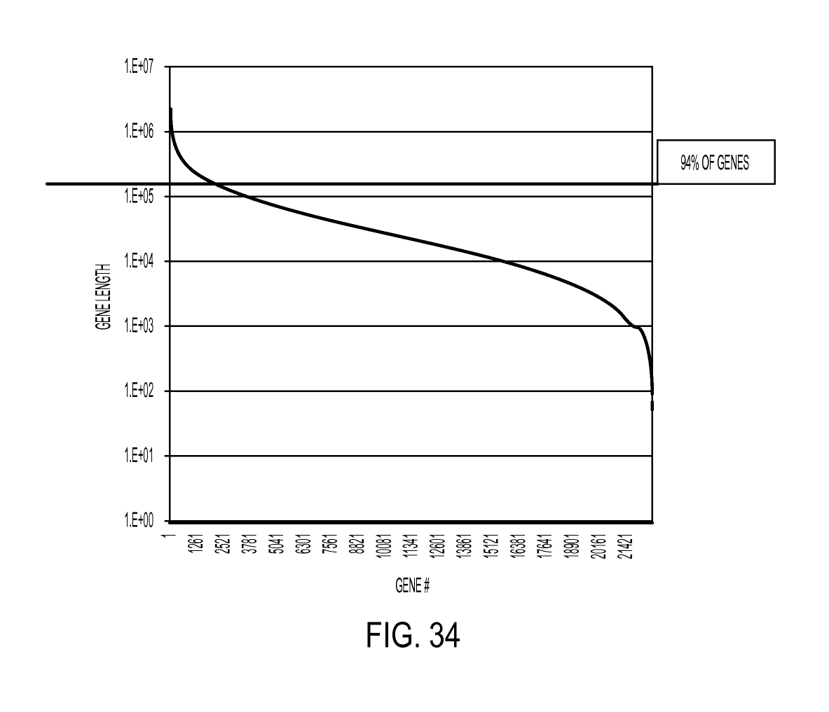

Linkage out to 210 kb can be measured in a single partition (e.g., well). FIG. 34 illustrates all the genes in the human genome sorted according to their length, as measured from the start codon to the stop codon. 94% of the genes are shorter than 210 kb. Methods described herein can be used for phasing variants in human genes.

While preferred embodiments have been shown and described herein, it will be obvious to those skilled in the art that such embodiments are provided by way of example only. Numerous variations, changes, and substitutions will now occur to those skilled in the art without departing from the methods and compositions described herein. It should be understood that various alternatives to the methods and compositions described herein can be employed. It is intended that the following claims define the scope of the invention and that methods and structures within the scope of these claims and their equivalents be covered thereby.

1. A method for determining an arrangement of at least three loci on a chromosome, the method comprising:
a) obtaining a sample comprising polynucleotide fragments of the chromosome;
b) partitioning the polynucleotide fragments of the chromosome to form isolated partitions;
c) amplifying at least three loci from the polynucleotide fragments of the chromosome in the isolated partitions, thereby generating at least three amplified loci of the chromosome;
d) detecting the at least three amplified loci of the chromosome in individual isolated partitions with a set of at least three probe;
e) determining linkage frequencies among the at least three loci of the chromosome based on the step of detecting; and
f) based on the linkage frequencies, determining the arrangement of the at least three loci on the chromosome.

2. The method of claim 1, the at least three loci including a first locus, a second locus, and a third locus, wherein the step of determining the arrangement of the at least three loci comprises a step of determining a distance between the first locus and the second locus, a step of determining a distance between the first locus and the third locus, and a step of determining a distance between the second locus and the third locus.

3. (canceled)

4. (canceled)

5. The method of claim 2, wherein each distance is a relative distance.

6. The method of claim 2, wherein the distances are determined by comparing the linkage frequencies to a standard.

7. (canceled)

8. The method of claim 1, wherein the step of determining the arrangement of the at least three loci comprises determining an order of a first locus, second locus, and a third locus on the chromosome.

9. The method of claim 1, further comprising detecting a plurality of amplified loci of the chromosome with a second set of at least three probes, wherein the at least three probes of the first set anneal to a first locus, a second locus, and a third locus, and wherein the at least three probes of the second set anneal to the second locus, the third locus, and a fourth locus that is not included in the at least three loci but do not anneal to the first locus.

10.-23. (canceled)

24. The method of claim 1, further comprising a step of detecting an amplified reference locus in the individual isolated partitions with a reference probe, wherein the reference locus is not substantially linked in the sample to the at least three loci of the chromosome, and also comprising a step of determining a copy number of each of the at least three loci based on comparing a quantity of each of the three loci with a quantity of the reference locus.

25.-34. (canceled)

35. The method of claim 1, further comprising performing next generation sequencing on a sample comprising the chromosome to produce next generation sequencing data on the chromosome.

36. The method of claim 5, wherein determining the arrangement of the at least three loci comprises inputting the linkage frequencies and next generation sequencing data into a computer implemented algorithm.

37. The method of claim 35, wherein the next generation sequencing data comprises data on one or more chromosome breakpoints.

38.-51. (canceled)

52. The method of claim 1, wherein determining linkage frequencies comprises measuring a difference between an observed number of partitions that comprise co-localized loci versus an expected number of partitions that comprise co-localized loci due to random Poisson-based distribution of two independently segregating loci.

53. (canceled)

54. The method of claim 1, wherein linkage frequency is dependent on a degree of fragmentation of the polynucleotides in the sample, and wherein a higher degree of fragmentation yields a lower linkage frequency.

55.-70. (canceled)

71. The method of claim 1, wherein determining linkage frequencies comprises comparing an abundance of partitions positive for a first locus and a second locus with an abundance of partitions positive for the first locus and not the second locus.

72. (canceled)

73. The method of claim 1, wherein the at least three loci comprise loci A, B, and C, and wherein the following populations of partitions are generated: partitions with no loci; partitions with individual loci A, B, or C; partitions with only loci A and B; partitions with only loci B and C; and partitions with only loci A and C.

74.-146. (canceled)

147. A method for determining a distance between a first locus and second locus on a first polynucleotide, the method comprising
a) partitioning a sample comprising the first and second locus into a plurality of partitions;
b) determining a number of partitions that comprise the first locus but not the second locus;
c) determining a number of partitions that comprise the second locus but not the first locus;
d) determining a number of partitions that comprise the first locus and the second locus;
e) determining a number of partitions that comprise neither the first locus nor the second locus;
f) determining, based on the numbers in steps b-e, a linkage frequency of the first locus and second locus in the sample; and
g) based on the linkage frequency, determining a distance between the first locus and second locus on the first polynucleotide.

148.-174. (canceled)

175. The method of claim 1, further comprising repeating steps (b) through (f) using another set of at least three probes that detects at least two of the at least three loci and an additional locus not included in the at least three loci, to map the additional locus relative to the at least three loci.

176. The method of claim 175, wherein each set of at least three probes includes a same set of at least three different labels.

177. The method claim 175, further comprising a step of determining a copy number of each of the at least three loci based on data collected from the isolated partitions.
